SEQUENCE LISTING (49 sequences)

SEQ ID NO: 1 - protein, 17 aa, Gallus sp.
  Ovalbumin peptide
ISQAVHAAHAEINEAGR

SEQ ID NO: 2 - protein, 16 aa, Homo sapiens
  HSV-1 glycoprotein D peptide
APYTSTLLPPELSETP

SEQ ID NO: 3 - protein, 7 aa, Artificial Sequence
  Description of Artificial Sequence Synthetic peptide
MSGGGGC

SEQ ID NO: 4 - protein, 8 aa, Artificial Sequence
  Description of Artificial Sequence Synthetic peptide
GGGGSGGG

SEQ ID NO: 5 - protein, 17 aa, Artificial Sequence
  Description of Artificial Sequence Synthetic peptide
KAERADLIAYLKQATAK

SEQ ID NO: 6 - DNA, 58 bp, Artificial Sequence
  Description of Artificial Sequence Synthetic oligonucleotide
ttaagtaaggcagaacgagcagatctaatagcttatctaaaacaagccaccgcgaagg

SEQ ID NO: 7 - DNA, 58 bp, Artificial Sequence
  Description of Artificial Sequence Synthetic oligonucleotide
ctagccttcgcggtggcttgttttagataagctattagatctgctcgttctgccttac

SEQ ID NO: 8 - DNA, 58 bp, Artificial Sequence
  Description of Artificial Sequence Synthetic oligonucleotide
cttaagtagcgctagcggagggggcggaagcggcggaggggcaagcttcagaccatgg

SEQ ID NO: 9 - DNA, 53 bp, Artificial Sequence
  Description of Artificial Sequence Synthetic oligonucleotide
cccgaattctattagcagcctcctccacctgacattgggaggagggttttctc

SEQ ID NO: 10 - DNA, 26 bp, Artificial Sequence
  Description of Artificial Sequence Synthetic oligonucleotide
ccactagtccactcgaccgtgacagg

SEQ ID NO: 11 - DNA, 33 bp, Artificial Sequence
  Description of Artificial Sequence Synthetic oligonucleotide
ggttcctcgagtatcaaagaggaacacaccatc

SEQ ID NO: 12 - DNA, 24 bp, Artificial Sequence
  Description of Artificial Sequence Synthetic oligonucleotide
ggaggggcaagcttcagaccatgg

SEQ ID NO: 13 - DNA, 27 bp, Artificial Sequence
  Description of Artificial Sequence Synthetic oligonucleotide
tgacattgggaggagggttttctcttc

SEQ ID NO: 14 - DNA, 75 bp, Artificial Sequence
  Description of Artificial Sequence Synthetic oligonucleotide
gtacgaattctattattcgtgccattcgattttctgagcttcgaagatgtcgttcaggcctcctccacctgacat

SEQ ID NO: 15 - protein, 18 aa, Artificial Sequence
  Description of Artificial Sequence Biotin ligase BirA target sequence
GGGSLNDIFEAQKIEWHE

SEQ ID NO: 16 - DNA, 52 bp, Artificial Sequence
  Description of Artificial Sequence Synthetic oligonucleotide
atcgccccctacaccagcaccctgctgccccccgagctgagcgagacccccg

SEQ ID NO: 17 - DNA, 56 bp, Artificial Sequence
  Description of Artificial Sequence Synthetic oligonucleotide
ctagcgggggcttcgctcagctcggggggcagcagggtgctggtgtagggggcgat

SEQ ID NO: 18 - DNA, 51 bp, Artificial Sequence
  Description of Artificial Sequence Synthetic oligonucleotide
caccgcgtcgcgacagctacaggtgtccactccatctctcaggctgttcac

SEQ ID NO: 19 - DNA, 60 bp, Artificial Sequence
  Description of Artificial Sequence Synthetic oligonucleotide
caccgcgtcgcgacagctacaggtgtccactccgccccctacaccagcaccctgctgccc

SEQ ID NO: 20 - DNA, 45 bp, Artificial Sequence
  Description of Artificial Sequence Synthetic oligonucleotide
caccgcgaattcgctccctgacatgggggctggaatctcaggttc

SEQ ID NO: 21 - DNA, 39 bp, Artificial Sequence
  Description of Artificial Sequence Synthetic oligonucleotide
caccgcgaattcgctccctgggagagggcttggagcatc

SEQ ID NO: 22 - DNA, 39 bp, Artificial Sequence
  Description of Artificial Sequence Synthetic oligonucleotide
caccgcgcgtacgtcttgtcctacgacgagaaccccgtg

SEQ ID NO: 23 - DNA, 38 bp, Artificial Sequence
  Description of Artificial Sequence Primer
ggggggccatggctcgagtcagtagcaggtgccagctg

SEQ ID NO: 24 - DNA, 30 bp, Artificial Sequence
  Description of Artificial Sequence Primer
ggggggatccgtgattgctgagctgcctcc

SEQ ID NO: 25 - DNA, 27 bp, Artificial Sequence
  Description of Artificial Sequence Primer
gggccatggctctgcagatccccagcc

SEQ ID NO: 26 - DNA, 27 bp, Artificial Sequence
  Description of Artificial Sequence Primer
gggggatccactagcccgggaccagtg

SEQ ID NO: 27 - DNA, 32 bp, Artificial Sequence
  Description of Artificial Sequence Synthetic oligonucleotide
gaggtgtcgcgagagcaggtggagcagcttcc

SEQ ID NO: 28 - DNA, 28 bp, Artificial Sequence
  Description of Artificial Sequence Synthetic oligonucleotide
gtggaggaattcgtctgctcggccccag

SEQ ID NO: 29 - DNA, 30 bp, Artificial Sequence
  Description of Artificial Sequence Synthetic oligonucleotide
gaggtggttaacgatcccaaactctgctac

SEQ ID NO: 30 - DNA, 46 bp, Artificial Sequence
  Description of Artificial Sequence Synthetic oligonucleotide
gaggtgatcgataagtgtacttacgtttttagcgagggggcagggc

SEQ ID NO: 31 - DNA, 48 bp, Artificial Sequence
  Description of Artificial Sequence Synthetic oligonucleotide
gaggtggttaacgatcccaaactctgctacttgctagatggaatcctc

SEQ ID NO: 32 - DNA, 37 bp, Artificial Sequence
  Description of Artificial Sequence Primer
ggggaagcttttttcatttacgaacatctgtgaaggc

SEQ ID NO: 33 - DNA, 37 bp, Artificial Sequence
  Description of Artificial Sequence Primer
ggggtcgagttatcagatgagattatggctcttctgc

SEQ ID NO: 34 - DNA, 58 bp, Artificial Sequence
  Description of Artificial Sequence Primer
ggggctcgagttatcagcgctgttggtgccggcacctgacacagcagaggatgcagag

SEQ ID NO: 35 - protein, 20 aa, Artificial Sequence
  Description of Artificial Sequence Synthetic peptide
YDENPVVHFFKNIVTPRTPP

SEQ ID NO: 36 - DNA, 30 bp, Artificial Sequence
  Description of Artificial Sequence Primer
aagcagtggtaacaacgcagagtacgcggg

SEQ ID NO: 37 - DNA, 27 bp, Artificial Sequence
  Description of Artificial Sequence Primer
tttttttttttttttttttttttttdn

SEQ ID NO: 38 - DNA, 44 bp, Artificial Sequence
  Description of Artificial Sequence Oligonucleotide primer
ctaatacgactcactatgggcaagcagtggtaacaacgcagagt

SEQ ID NO: 39 - DNA, 21 bp, Artificial Sequence
  Description of Artificial Sequence Oligonucleotide primer
ctaatacgactcactatgggc

SEQ ID NO: 40 - DNA, 25 bp, Artificial Sequence
  Description of Artificial Sequence Oligonucleotide primer
atcctttctcttgaccatggccatc

SEQ ID NO: 41 - DNA, 24 bp, Artificial Sequence
  Description of Artificial Sequence Oligonucleotide primer
cacagcgacctcgggtgggaacac

SEQ ID NO: 42 - DNA, 25 bp, Artificial Sequence
  Description of Artificial Sequence Oligonucleotide primer
gctggaccacagccgcagcgtcatg

SEQ ID NO: 43 - DNA, 24 bp, Artificial Sequence
  Description of Artificial Sequence Oligonucleotide primer
cagctggtacacggcagggtcagg

SEQ ID NO: 44 - DNA, 35 bp, Artificial Sequence
  Description of Artificial Sequence Primer
tatggtcgacaagcttcacagagggcctggtctgg

SEQ ID NO: 45 - DNA, 41 bp, Artificial Sequence
  Description of Artificial Sequence Primer
tatggtcgacggtaccccagaaaagacctccagaaaatagc

SEQ ID NO: 46 - DNA, 31 bp, Artificial Sequence
  Description of Artificial Sequence Primer
ggtggttaacgtctgctctaccccaggcctc

SEQ ID NO: 47 - DNA, 36 bp, Artificial Sequence
  Description of Artificial Sequence Primer
ggtggttaacggaactttctgggctggggaagaagg

SEQ ID NO: 48 - DNA, 47 bp, Artificial Sequence
  Description of Artificial Sequence Primer
tatggtcgacggtacccttcatgttaaggatcaagaccattatttgg

SEQ ID NO: 49 - protein, 20 aa, Homo sapiens
YDENPVVHFFKNIVTPRTPP

(19) United States
(12) Patent Application Publication
(10) Pub. No.: US 2004/0253632 A1
(43) Pub. Date: Dec. 16, 2004

(54) Modulation of T -cell receptor interactions

(75) Inventors: Peter R. Rhode, Miami, FL (US); Vaughan Wittman, Weston, FL (US); Jon A. Weidanz, Amarillo, TX (US); Martin Burkhardt, Miami, FL (US); Kimberlyn F. Card, Pembroke Pines, FL (US); Rony Tal, Coral Springs, FL (US); Jorge Acevedo, Thousand Oaks, CA (US); Hing C. Wong, Weston, FL (US)

Correspondence Address: EDWARDS & ANGELL, LLP, P.O. BOX 55874, BOSTON, MA, 02205, US

(73) Assignee: Sunol Molecular Corporation

(21) Appl. No.: 09/859,012

(22) Filed: May 16, 2001

Related U.S. Application Data
(60) Provisional application No. 60/206,920, filed on May 25, 2000.

(51) Int. Cl. G01N 33/53; G01N 33/567

(52) U.S. Cl. 435/7.1; 435/7.2

(57) ABSTRACT

Disclosed are methods for identifying compounds that modulate an immune complex that includes a T cell receptor (TCR) and a major histocompatibility complex (MHC) antigen. The invention has many useful applications including providing high throughput screening assays for detecting compositions that can modulate an immune response.

### CROSS-REFERENCE TO RELATED APPLICATION

The present application is a continuation-in-part of U.S. Provisional Patent Application No. 60/206,920 filed on May 25, 2000, the disclosure of which is incorporated herein by reference.

### FIELD OF THE INVENTION

The present invention relates to the compounds that modulate interactions between a T cell receptor (TCR) and a major histocompatibility complex (MHC) antigen. In one aspect, the invention features methods for identifying the test compounds. In other aspects, the invention provides test compounds detected and identified by the methods, pharmaceutical preparations that include the compounds, as well as methods for using those compounds therapeutically. The invention has many important applications including use in high throughput screening formats to select compounds that can reduce or eliminate harmful immune responses or that can enhance or initiate helpful immune responses.

### BACKGROUND

Antigen-specific T cells recognize and respond to specific peptides bound to the binding groove or cleft of a major histocompatibility complex (MHC, including MHC of humans known as HLA). The presentation of peptides by the MHC molecule and the recognition by antigen-specific T cells represent an important immunosurveillance strategy for identifying and responding to foreign antigens. The antigenic peptides are non-covalently bound to particular “binding pockets” comprised of polymorphic residues of the MHC protein's binding groove. The MHC-bound antigenic peptides specifically interact with T cell receptors (TCRs) on the surface of the T cells and modulate an immune response. See generally Fundamental Immunology 3rd Ed., W. Paul Ed. Rsen Press LTD. New York (1993).

See also U.S. Pat. No. 5,869,270 to Rhode, P. et al. (assigned to Sunol Molecular Corporation).

MHC molecules are membrane-bound heterodimeric glycoproteins that include an α and β chain. For class II MHC molecules, the α1 and β1 domains associate to form a peptide binding grove. Antigenic peptides bind the MHC molecule through interaction between anchor amino acids on the peptide and the binding pockets formed by the α and β domains. The x-ray crystallographic structure of a human class II HLA-DR1 complexed with an influenza virus peptide has show in atomic detail the interactions between the peptide and the MHC molecule. See Brown, J. H. et al. (1993) Nature 364: 33-39; Stem, L. J. et al. (1994) Nature 368: 215-221; and Garboczi, D. N. et al. (1999) Immunity 10: 1-7.

MHC class I molecules have different domain organizations than MHC class II molecules. In class I MHC molecules, the α1 and α2 domains associate to form the peptide 1 binding groove. However both classes of MHC molecules have overall similar structure with a peptide binding site or groove that is distal to membrane domains. See e.g., Rudensky, A. Y. et al., (1991) Nature 353:622-626. See also U.S. Pat. Nos. 5,284,935; 5,260,422; 5,194,425; 5,130,297; and WO 92/18150, WO 93/10220 for discussions of MHC molecules.

The T-cell response is modulated by antigen binding to a T-cell receptor (TCR). One type of TCR is a membrane bound heterodimer consisting of an α and β chain. This TCR is thought to resemble an immunoglobulin variable (V) and constant (C) region. The TCR α chain includes a covalently linked V-α and C-α chain, whereas the β chain includes a V-β chain covalently linked to a C-β chain. The V-α and V-β chains form a pocket or cleft that can bind to superantigen or peptide antigen in the context of the MHC molecule or to an alloreactive MHC molecule. See generally Paul, W., supra.

It has been reported that if TCR α and β chains are not assembled into a complex that includes a particular cluster of differentiation (CD) protein, i.e., a CD3 complex comprising γ, δ, , and β chain s, the TCR can be degraded. See e.g., Shin et al. (1993) Science 259: 1901.

There is recognition that MHC molecules complexed with antigenic peptides can induce selective immunosuppression by several different mechanisms. See e.g., Guery, J. et al. (1993) Critical Reviews in Immunology 13(3/4): 195-206.

More specifically, it has been reported that peptide-MHC complexes on the surface of antigen presenting cells (APCs) will only induce clonal expansion of a T cell line specific for the MHC bound peptide if the antigen presenting cells also deliver co-stimulatory signals. One proposed approach takes advantage of this requirement for T cell activation and inhibits T cell development by interaction with the antigenic peptide bound to the MHC molecule in the absence of co-stimulatory signals. See Nicolle, M. et al., J. Clin. Invest. (1994) 93: 1361-1369; and Sharma, S., et al., Proc. Natl. Acad. Sci. USA (1991) 88: 11465-11469.

There has been a substantial effort toward understanding the immune complex formed between the TCR and its compatible (cognate) MHC. One approach has been to use X-ray crystallography to study the complex at the atomic level. See Garboczi, D. N., et al. (1996) Nature 384:134-141, Garcia, K. C., et al. (1998) Science 279:1166-1172, Ding, Y. H., et al. (1998) Immunity 8:403-411 and Reinherz, E. L., et al. (1999) Science 286:1913-1921. Using this approach, it has been reported that different TCR:MHC/peptide immune complexes feature similar binding interactions. These reports have been supported by mutagenesis studies. See Rabinowitz J. D. et al. (1996) Immunity 5:125-135, Lyons D. S. et al. (1996) Immunity 5:53-61, Brawley J. V. & Concannon P. (1999) J. Immunol. 163:4946-4952, Homell T. M. C. et al. (1999) J. Immunol. 163:3217-3225.

These efforts have further suggested that minor amino acid changes in either the antigenic peptide or the MHC molecule can produce MHC/peptide complexes that do not optimally stimulate T cell responses. For example, such complexes have been reported to be T cell antagonists and weak agonists. In some cases, changes in the antigenic peptide can result in MHC/peptide complexes that act as T cell super agonists.

There have been efforts toward understanding interactions between a TCR and its cognate MHC/peptide and the formation of an immune complex that leads to stimulation of T cell responses. For example, the stability of TCR:MHC/peptide immune complexes is important for optimal T cell responses. Oligomerization of multiple TCR:MHC/peptide immune complexes may also be important for optimal responses. See Lyons D. S. et al. (1996) Immunity 5: 53-61, Cochran, J. R. et al. (2000) Immunity 12: 241-250.

There have been efforts to develop efficient screening methods for detecting compounds using cell-based and protein-based protocols. For example, some of these screens have identified compounds that reportedly inhibit protein: protein interactions. See Tian, S. S. et al. (1998) Science 281: 257-259, Kelly T. A. et al. (1999) J. Immunol. 163: 5173-5177, Jenh, C. H. et al. (1998) Anal. Biochem. 256: 47-55.

It would be desirable to have compounds that can modulate interactions between a TCR and its cognate MHC. It would be further desirable to have efficient methods for detecting the compounds that are compatible with a wide variety of high throughput screening paradigms.

### SUMMARY OF THE INVENTION

The present invention generally relates to compounds that modulate interactions between a T cell receptor (TCR) and a major histocompatibility complex (MHC, including HLA in humans) antigen. In one aspect, the invention features methods for detecting compounds that can modulate interactions between the TCR and its cognate MHC antigen molecule.

More particularly, we have developed methods for detecting compounds that modulate TCR:MHC antigen interactions particularly by increasing or decreasing presence of the immune complex formed between such molecules. The word “modulate” as it is used in reference to a method of this invention, is intended to mean detected compounds that preferably act as agonists or antagonists of the immune complex and, more generally, as modulators of the TCR:MHC antigen complexes themselves.

The term “MHC antigen” or “MHC antigen molecule” is used in reference to an MHC-based molecule that specifically binds the TCR. The MHC component of the MHC antigen can consist of but is not limited to classical or non-classical MHC class Ia, class Ib or class II molecules or to MHC homologues (See Maenaka, K & Jones, E. Y. 1999. Curr. Opin. Struct.

Biol. 9:745-753). The MHC antigen can consist of but is not limited to MHC/peptide complexes, MHC/superantigen complexes, MHC/lipid (or gylocolipid) complexes or alloreactive (xenoreactive) MHC molecules.

The methods of this invention broadly involve contacting at least one TCR molecule with at least one cognate MHC antigen (as defined above) preferably in the presence or absence of at least one test compound. By the term “cognate” as it is used to reference a particular TCR or MHC antigen molecule is meant that molecule which serves as a specific binding partner for the other molecule. In many embodiments, the methods will include about one TCR molecule and about one cognate MHC antigen molecule. Typical contact between the molecules is usually conducted under pre-determined conditions that foster specific binding between the TCR and MHC antigen molecules. That specific binding generally assists formation of the immune 1 complex and may help stabilize that complex in some instances. Detected test compounds significantly increase or decrease that specific binding relative to at least one control which control can be performed at the same or different time than when the test compound was detected by the method. Compounds having a desired activity or more than one of such activities are thus identified in the method as being capable of modulating formation of the immune complex. The test compounds so identified can be further selected according to the invention. Such identified and selected compounds have many important applications including use as agents for modulating (increasing or decreasing) a wide spectrum of immune responses in vitro and in vivo. Illustrative of such immune responses include immune surveillance, auto-immune disorders, infection, proliferative disorders such as cancer, graft acceptance and the like.

Practice of the present invention provides many advantages particularly in light of prior screening attempts. For example, the methods of this invention are fully compatible with a wide range of TCR and MHC antigen molecules. This feature of the invention is important as it expands the repertoire of probe formats for detecting the test compounds. As an example, the methods of this invention can accommodate fully soluble TCR and MHC antigen molecules as well as less soluble molecules such as those expressed on cell surfaces. Accordingly, the invention is very flexible and can be used in a wide spectrum of different test formats, e.g., those that use fully soluble proteins, cell-expressed membrane proteins, or combinations thereof including screening strategies in which one of the molecules is fully soluble and the other is expressed by a desired cell. In marked contrast, prior screening attempts have employed more restricted screening formats where only one or a few molecule types have been employed.

Other advantages are provided by this invention. For example, in embodiments in which a particular method uses at least one class I or class II MHC antigen molecule, practice of the invention can eliminate the need to purify MHC molecules previously loaded with tightly bound peptides from antigen presenting cells (APCs). In contrast, prior screening attempts have reported use of MHC molecules prepared by difficult and time intensive purification steps. In contrast, the present methods are compatible with use of a wide range of class I or class II MHC antigen molecules that are much easier to make and use. See e.g., the U.S. Pat. No. 5,869,270 to Rhode, P. et al.; the disclosure of which is hereby incorporated by reference.

Additionally, many embodiments of the present methods offer more sensitivity than that reported in prior screening attempts. For example, it has been disclosed that a multi-valent array of MHC antigen complexes is necessary to stimulate certain TCR expressing cells. That is, it was reported that monovalent MHC antigen complexes would not work as good probes in many screening attempts. In marked contrast, the methods of this invention can be used to detect test compounds using a range of class I or class II MHC antigen molecules including monovalent and multi-valent molecules. See the discussion below and the U.S. Pat. No. 5,869,270 for disclosure relating to making and using such molecules.

Further, prior screening attempts have often focused cell expressed TCR heterodimers as probes. It is believed that this format is unnecessarily restrictive and may in some instances negatively impact screening results. In contrast, the methods of this invention are not limited to a particular TCR molecule or test format. Instead, the invention is fully compatible with broad spectrum of TCR molecules including, but not limited to, naturally-occurring TCRs, fully soluble TCR molecules including recombinant single chain constructs, as well as cell-anchored single chain and heterodimeric TCR molecules.

Additional flexibility of the invention is manifested by the wide spectrum of MHC antigen molecules that can be employed including naturally-occurring MHC molecules, fully soluble MHC molecules including, but not limited to, single chain constructs, as well as those constructs expressed by cells as cell surface molecules. Moreover, what is referred to herein as “empty” MHC molecules can also be used in accord with this invention.

Accordingly, practice of the present invention can substantially increase the pool of testable TCR and MHC antigen probe molecules. The considerable size of this pool also increases the number of testable immune complexes, thereby expanding the universe of test compounds that can be detected, identified and selected by the present methods. In contrast, prior screening attempts have been limited to one or only a few probe types, usually TCR and MHC heterodimers. This deficiency is believed to substantially reduce the potential number of detected compounds in many assay settings.

Still further, prior screening attempts have often been limited to detecting TCR:MHC-peptide interactions by using only one or a few limited methods. Typical of such methods involve relatively insensitive and often imprecise measurements of cell granulation. In marked contrast, the present invention provides a wide array of methods for detecting those interactions generally by monitoring the immune complex in the presence and absence of the test compounds. More specific detection methods offer more sensitive and reliable indicators of the TCR:MHC antigen interactions which methods can be conducted using direct or indirect techniques.

In particular, the capacity of this invention to accommodate a wide range of methods for detecting the immune complex provides notable advantages. One advantage is that such methods make the invention even more flexible and tailorable to a wide range of different screening paradigms.

For example, in embodiments in which highly sensitive detection of the immune complex is desirable, the invention can be used with indirect detection methods, e.g., to amplify weak or intermittent signals typical of TCR:MHC antigen interactions. Alternatively, in embodiments in which presence of the immune complex is especially robust, the invention can be employed using methods that involve direct detection of immune complex, its TCR and/or MHC antigen components, or the test compound. Such an embodiment may be very practical in settings in which rapid screening of test compounds is indicated. In contrast, prior methods for detecting TCR:MHC-peptide interactions as reported in prior screens has, until now, been much more restricted and generally less sensitive, thereby decreasing or even eliminating opportunities to detect many useful test compounds.

Additional advantages are provided by this invention.

For example, in some embodiments of this invention, the capacity of one or more test compounds to modulate an immune complex of interest can be compared against a variety of controls. An exemplary control is the detection of the immune complex in the absence of the test compounds. However in certain situations more refined controls may be indicated to select against those test compounds which non-specifically impact the immune complex. More particularly, the invention provides a variety of what is sometimes referred to as “internal controls” that can be used to help detect test compounds with capacity to specifically modulate subject TCR:MHC antigen interactions.

In one embodiment, such an internal control will include the subject (first) TCR or MHC antigen molecule and at least one antibody, usually a monoclonal antibody, that binds the TCR or MHC antigen molecule specifically. Particular antibodies of interest will bind the TCR or MHC antigen molecule generally outside the region involved in specific binding and formation of the immune complex. Other particular antibodies suitably bind, usually specifically, a CD protein closely associated with the TCR molecule typically in embodiments in which the TCR molecule is cell expressed.

In the foregoing example of the invention, the complex formed between the antibody and the TCR or MHC antigen molecule is referred to herein as a “control immune complex” or like phrase. Formation of the control immune complex in accord with this invention typically facilitates a detectable response in the method such as but not limited to a cell response. Such a response can be directly or indirectly measured apart from or along with the (experimental) immune complex, i.e., that complex formed by interaction between the subject (first) TCR and MHC antigen molecules of interest. A particular test compound will desirably modulate formation of the experimental immune complex but will impact the control immune complex much less and preferably not at all.

In another embodiment, the control immune complex includes a second TCR molecule and its cognate MHC antigen molecule preferably wherein the second TCR molecule has a different binding specificity than the first TCR molecule. In this example of the invention, particular test compounds of interest will modulate the immune complex formed between the subject (first) TCR and its cognate MHC antigen molecule. However, the test compounds will impact the control immune complex with the second TCR molecule and its cognate MHC antigen molecule much less and preferably not at all.

As discussed, presence of the immune complexes including the particular control immune complexes disclosed can be detected using a wide variety of detection formats including those relying on direct or indirect techniques. Practice of one or more of the internal controls can be conducted prior to, during, or after screening of the test compounds.

In embodiments of the invention in which use of one or more of the foregoing controls is preferred, substantial advantages can be realized. For example, methods employing such controls can offer much better sensitivity, selectivity and/or reliability. That is, in embodiments in which at least one control such as the internal controls discussed are used, presence of the control helps to facilitate the detection and selection of the test compounds, e.g., by identifying those which non-specifically interfere with TCR:MHC antigen interactions. This feature of the invention can often improve screens for test compounds that specifically modulate an immune complex of interest. In contrast, prior screening attempts have not always provided good controls and it is not clear whether such attempts can provide sensitive and reliable results.

Accordingly, it will be apparent that the invention is highly flexible and can be adapted to screen a wide range of test compounds. A preferred test compound in accord with this invention will exhibit one or more desired activities including capacity to modulate subject TCR:MHC antigen interactions.

For example, in embodiments in which the methods are adapted to detect antagonists, a suitable test compound will show at least about 10%, preferably at least about 20%, more preferably at least about 50%, and still more preferably at least about 75% to about a 100% decrease in the presence of the immune complex relative to at least one suitable control. As discussed, that immune complex is formed between the subject TCR and its cognate MHC antigen molecule. As also discussed, the presence of the immune complex can be readily detected using a variety of direct and indirect detection techniques. An illustrative control involves conducting the assay in the absence of the test compound. Alternatively, or in addition, the control can be one or more of the internal controls mentioned previously and in the discussion that follows.

However in embodiments in which invention is used to detect agonists, the assay employed will preferably show at least about a 10%, preferably at least about 20%, more preferably at least about 50%, and still more preferably at least about 75% to about 100% up to about a 500% to about 1000% increase in the presence of the immune complex relative to at least one suitable control.

As pointed out, the invention can be used in a wide range of formats. For example, the invention can be employed in what is known as “high throughput” or “ultra throughput” type screening methods. In this embodiment, the invention is particularly useful for screening large pools of test compounds including libraries of same for those particular compounds with capacity to modulate the immune complex.

Further, the methods of this invention can be used in combination with one or more in vivo screening regimens to determine more precisely the capacity of detected test compounds to modulate subject TCR:MHC-peptide interactions. For example, in one embodiment, the invention can be used to pre-screen (or concurrently screen) suitable test compounds for use in a mammal such as those recognized animal models used to identify compounds impacting immune surveillance, auto-immune disorders, infection, proliferative disorders such as cancer, and/or graft acceptance. The monitored function in the animal may be pre-existing e.g., a genetic predisposition, or it may be induced, e.g., chemically or surgically.

Significantly, the present invention contemplates multiple detection formats (e.g., a combination of in vitro and in vivo assays) to facilitate the detection of highly useful test compounds that can modulate TCR:MHC antigen interactions and particularly the presence of desired immune complexes. This feature of the invention greatly extends the flexibility of the invention and provides for more specific selection and identification of useful test compounds.

Such broad spectrum testing, by the in vitro and/or in vivo methods, provides additional advantages. Thus, for example, many of the methods can be used to perform multiple analyses, thereby enhancing the efficiency and probability of identifying test compounds that can specifically modulate subject immune complexes and exhibit substantial therapeutic capacity. This feature of the invention is especially useful when large numbers of compounds need to be tested such as when high throughput or ultra throughput methods are used. For instance, libraries of test compounds can be made by standard synthetic methods including combinatorial-type chemistry manipulations and then tested in accord with the invention. Alternatively, the methods of this invention can be used with test compounds already available such as small molecules, large molecules, including compounds present in commercially available chemical libraries.

Accordingly, in one aspect, the invention features methods for identifying compounds that modulate TCR:MHC antigen interactions. In one embodiment, the method includes at least one of and preferably all of the following steps:

a) contacting a first TCR molecule and a class I or class II MHC antigen molecule in the presence or absence of at least one test compound, the contacting being under conditions sufficient to bind the TCR and the MHC antigen molecules specifically as an immune complex,

b) detecting presence of the immune complex in the presence and absence of the test compound; and

c) selecting a test compound that alters specific binding between the TCR and MHC antigen molecules. Preferably, the method further includes identifying the selected compound as being capable of modulating the immune complex.

In a particular embodiment of the method, the invention provides a screen for identifying test compounds that modulate the immune complex that includes the TCR and its cognate MHC antigen. In a more specific embodiment, the method includes at least one and preferably all of the following steps:

a) contacting cells expressing a first TCR molecule including T cell hybridomas with an class I or class II MHC antigen molecule bound to a solid support in the presence or absence of a test compound, the contacting being under conditions sufficient to bind the TCR and the bound MHC antigen molecule specifically as an immune complex,

b) detecting presence of the immune complex in the presence and absence of the test compound by measuring a cell response from the cells; and

c) selecting a test compound that alters specific binding between the first TCR and bound MHC antigen molecules. Preferably, the method further includes identifying the selected compound as being capable of modulating the immune complex.

As discussed, the methods of this invention can be used to screen for desired test compounds using one or a combination of strategies. As also discussed, it will often be very useful to include, as a control, one or more steps that detect the immune complex in the absence of the test compound. However in embodiments in which presence of the immune complex is already understood in a particular assay format, such steps can be omitted if desired.

In other embodiments, the control may include, alternatively or in addition to detecting the immune complex without the test compound, at least one suitable internal control. In embodiments in which use of at least one internal control is indicated, the method will generally include steps for detecting presence of the control immune complex in the presence and absence of the test compound. Choice of whether to include the foregoing controls will be guided by understood parameters such as the TCR and MHC antigen molecules used, the assay format selected and sensitivity required.

In another aspect, the present invention provides pharmaceutical compositions that preferably include at least one of the compounds detected and identified by the present methods. Such compositions may include the compounds as the sole therapeutic agent or may combine same with one or more other recognized agents including drugs having acknowledged effects on immune system function.

The present invention also provides methods for inhibiting an immune response in a mammal. In one embodiment, the method includes administering a therapeutically effective amount of at least one of the pharmaceutical compounds provided herein. The methods include, but are not limited to, administration of at least one of the pharmaceutical compounds as the sole active agent. In another embodiment, the methods provide for administration of at least one of the pharmaceutical compounds in combination with at least one other recognized agent such as those drugs with acknowledged therapeutic effects on the immune system.

In another aspect, the invention provides kits and particularly diagnostic or research kits for performing the methods disclosed herein. A particular kit of this invention includes, preferably as a packaged combination, a first container that includes at least one TCR molecule or a functional fragment thereof; and a second container that includes at least one MHC antigen molecule or a functional fragment thereof. Optionally, the kit may further include one or more buffers for performing the methods as well as directions for using the kit. Also optionally, the kit may include one or more of the controls described herein. In one embodiment, the controls include at least one naturally-occurring, synthetic or semi-synthetic compound with a recognized capacity to modulate specific binding between an identified TCR molecule and an identified MHC antigen molecule; or functional fragments thereof. Preferably, the compound decreases that specific binding.

Additionally provided by this invention are recombinant TCR molecules that include a mammalian CD3ζ (zeta) sequence; or functional fragment thereof. Such molecules are, in one embodiment, single chain constructs that can be functionally positioned in cell membranes.

### BRIEF DESCRIPTION OF THE DRAWINGS

FIG. 1A-B are drawings showing interactions between various T cell receptors (TCRs) and cognate major histocompatibility (MHC) peptide complexes.

FIG. 2 is a schematic drawing showing one method of the invention for detecting test compounds. In this example, a small molecule inhibitor inhibits TCR/MHC antigen interactions and helps to block undesirable immune responses.

FIG. 3 is a schematic drawing showing a particular protein-based method for detecting test compounds that block autoimmune disorders.

FIG. 4 is a schematic drawing showing a particular cell-based method for detecting test compounds that block autoimmune responses.

FIG. 5A is a schematic drawing showing a “proof of principle” example of the invention in which a chemical library is screened for inhibitors of DO11.10 TCR and scIA^{d}/OVA interactions.

FIG. 5B is a schematic drawing showing another “proof of principle” example of the invention in which a high throughput screening format is used to detect inhibitors of DO11.10 TCR and sc-IA^{s}/OVA interactions. Detected inhibitors are identified by differences in optical absorbance compared to internal controls.

FIG. 6A is a schematic drawing showing a specific example of a method for detecting test compounds that inhibit multiple sclerosis (MS).

FIG. 6B is a schematic drawing showing a specific method for detecting test compounds in which a high throughput screen is used. In the figure, the method is optimized to detect molecules that inhibit multiple sclerosis (MS).

FIG. 7A-C are drawings showing steps conducted for IE^{k} cloning. FIG. 7A: Genetic constructs containing various α and β chain fragments. FIG. 7B: Construction of single chain IE^{k} molecules with linked peptide. FIG. 7C: Insect cell expression vectors including IE^{k} single chain. Abbreviations used in each of the figures are defined in FIG. 7C.

FIG. 8 is a photographic representation of an SDS-PAGE gel showing the sc-IA^{d}-IgG molecule in reduced (lanes 1-2) and non-reduced (lanes 3-4) form. Lane 5 shows protein size markers. Lane numbers are from left to right.

FIG. 9 a photographic representation of an SDS-PAGE gel showing the DO111.10 sc-TCR-IgG2 b fusion. Lane 1 shows an antibody control and lane 2 shows the DO11.10 scTCR-IgG2b fusion protein.

FIG. 10 is a schematic drawing showing various TCR-CD3ζ fusion constructs of the invention.

FIG. 11 is a graph showing the results of a fluorescence activated cell sorting (FACS) experiment in which BW5147 transfectants expressing various DO11.10 scTCR-CD3ζ, fusion constructs were stained with an anti-TCR antibody probe.

FIGS. 12A and 12B are graphs showing FACS characterization of BWDZ:CD4+transfectants. FIG. 12A: Detection of surface CD4 expression by FACS. FIG. 12B: Detection of surface DO11.10 scTCR expression by FACS.

FIG. 13 is a graph showing results of stimulation of BWDZ:CD4+cells by peptide pulsed antigen presenting cells (APCs).

FIG. 14 is a graph showing results of inhibition of IL-2 production in a DO11.10 T cell stimulation assay.

FIGS. 15A and 15B are graphs showing results of titration of two detected test compounds 180B 10 (FIG. 15A) and 29D11 (FIG. 15B).

FIG. 16 is a drawing showing illustrative inhibitory compounds.

FIGS. 17A and 17B are graphs showing results of stimulation of DO11.10 sc-TCR 2B4 transfectants by peptide-pulsed APCs. FIG. 17A shows a T cell stimulation assay using OVA peptide-loaded A20 APCs. FIG. 17B shows a T cell stimulation assay using PCC peptide-loaded CH12 cells.

FIG. 18 is a graph showing results of stimulation of DO11.10 scTCR 2B4 transfectants by NDO APCs.

FIG. 19 is a graph showing results of stimulation of DO11.10 sc-TCR-CD3ζ fusion transfected 2B4 T cell hybridomas.

FIG. 20 is a graph illustrating T cell staining using multi-valent MHC/peptide molecules.

FIG. 21 is a graph showing ELISA values for HLA-A2/264 peptide and 264 sc-TCR-kappa interactions.

FIG. 22A is a table showing specificity of response of a protein-based ELISA assay. The information is displayed graphically in FIG. 22B.

FIG. 23A is a table showing concentration dependence of protein ELISA. The information in the table is displayed graphically in FIG. 23B.

FIGS. 24A and 24B are graphs showing comparison of different TCR reagents in protein ELISA. The information in the graphs is shown in FIGS. 24C and 24D, respectively.

### DETAILED DESCRIPTION OF THE INVENTION

As discussed above, the present invention features methods for detecting compounds that can modulate interactions between a TCR and its cognate MHC antigen molecule. In particular, the invention provides highly useful screening methods for detecting and identifying the compounds. Further provided are compounds identified and selected by the methods, pharmaceutical compounds or preparations that include at least one of the selected test compounds, as well as methods for using those preparations therapeutically to treat an immune disorder in a human patient.

As also discussed, the invention is broadly applicable to what has been referred to as “high throughput” or “ultra throughput” screening paradigms. This embodiment of the invention is especially useful for manipulating and testing large pools of test compounds for those with good capacity to modulate TCR and MHC antigen interactions. More particular compounds identified by the methods of this invention can reduce or eliminate harmful immune responses triggered by unwanted TCR and MHC antigen interactions. Alternatively, such pharmaceutical compounds can be employed prophylactically to help eliminate, reduce the severity, or delay the onset of the immune disorder in the patient. Particular patients have or are suspected of having the immune disorder including those “at risk” for developing that disorder such as those with a pre-determined genetic disposition to such a disorder.

There has been recognition that modest amino acid changes within an MHC molecule or its antigenic (presenting) peptide, even conservative amino acid changes, can provide MHC/peptide complexes that are inactive or act as antagonists, weak antagonists, or super agonists with respect to stimulating T cell responses. Similar modest amino acid changes in the TCR have been shown to alter T cell specificity to peptide and MHC. See, for example, Rabinowitz J. D. et al. (1996) Immunity 5:125-135, Brawley J. V. & Concannon P. (1999) J. Immunol. 163:4946-4952, Hornell T. M. C. et al. (1999) J. Immunol. 163:3217-3225. Chemical compounds that can interact with the MHC antigen complex or the TCR may alter the interaction with the cognate receptor and may similarly act as T cell antagonists or agonists.

Moreover, available X-ray crystallographic studies have suggested that there are significant amino acid interactions between the TCR and conservative and polymorphic side chains on the MHC. Further these studies have suggested that there are interactions between the TCR and the side chains of the peptide, and the peptide backbone of both the MHC and the peptide. Without wishing to be bound to any theory, it is believed that the present invention is well-suited to detect compounds that can impact and especially block or enhance those interactions. More preferably, such compounds can serve as specific antagonists or agonists of the immune complex formed between the TCR and MHC antigen in vitro and in vivo.

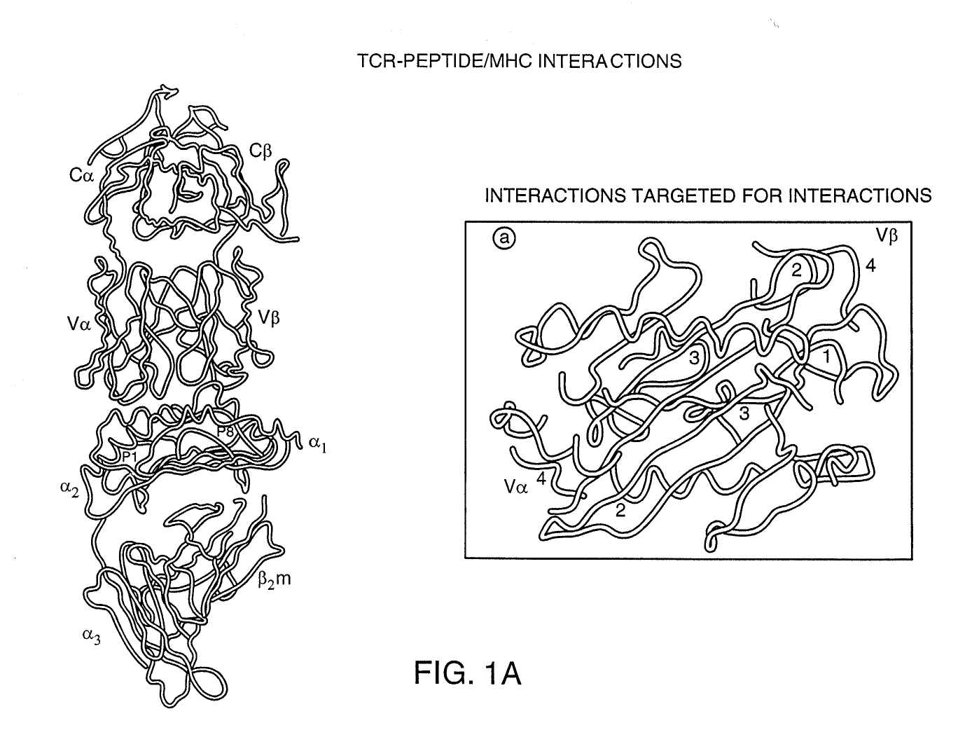

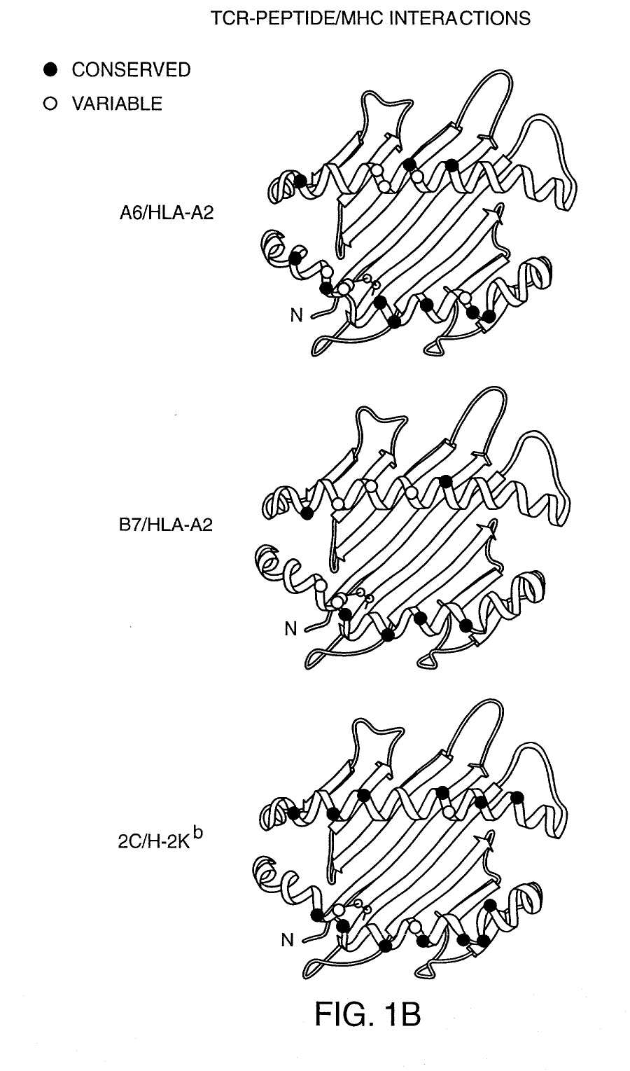

By way of illustration and not limitation, FIGS. 1A-B show some important TCR-peptide/MHC interactions. Many of these interactions exemplify relatively low affinity binding between the TCR and MHC antigen molecules. In particular, FIG. 1A shows the overall peptide backbone structure of one entire TCR:MHC/peptide complex (right panel) and a view of the peptide/MHC combining site with the CDRs (numbered) of the TCR superimposed onto the MHC/peptide structure (left panel) (See Garboczi, D. N., et al. (1996) Nature 384:134-141, Garcia, K. C., et al. (1998) Science 279:1166-1172). FIG. 1B shows conserved and variable MHC residues contacted by different cognate TCRs (See Garboczi, D. N. & Biddison, W. E. (1999) Immunity 10:1-7). Without wishing to be bound to theory, the present invention can take advantage of these interactions by detecting test compounds with capacity to modulate these and other low affinity interactions between the TCR and MHC antigen molecules.

The information provided by FIGS. 1A-B has facilitated understanding about multiple targets within the TCR:MHC antigen complex. This understanding has helped implementation of the present invention to detect and identify test compounds that modulate those targets. Many of the compounds detected by the methods may act very specifically, e.g., in a peptide-specific, MHC allele specific, MHC family-specific or MHC class-specific manner.

In addition, compounds that interact with the TCR:MHC antigen complex may alter the stability of the complex or the ability of the complex to multimerize. These compounds may act as T cell antagonists or agonists.

As mentioned previously, in one embodiment the invention can detect compounds that antagonize T cell activation, e.g., by specific MHC alleles or MHC families. This feature of the invention can provide important advantages including paving the way for the development and implementation of a wide range of therapeutic strategies. Such approaches include those optimized to treat an immune disorder including recognized autoimmune diseases. Significantly, many autoimmune diseases are acknowledged to have a strong genetic association with specific MHC alleles and/or MHC families. Thus it is another object of this invention to provide methods that can be tailored to particular patients or patient groups to detect test compounds that modulate (e.g., reduce or block) the TCR and MHC interactions in an MHC allele and/or family specific manner.

As indicated above, the formation of the immune complex is exemplified by a complex array of low affinity yet specific interactions between the TCR and the MHC antigen molecule. These and other factors can limit use of methods for studying high affinity receptor-ligand interactions and assessing formation of the TCR:MHC antigen complex. These limitations include the lack of sensitivity, difficulties in determining the specificity of the interactions and high assay variability. A number of features of the invention address these limitations. As described more fully below, the invention employs suitable protein- and cell-based reagents to boost the sensitivity and decrease the variability of the assay to detect the formation of the immune complex. In a particular embodiment, the invention also includes the use of suitable internal controls. As described below and in the Examples, internal controls employing non-cognate TCR or MHC antigens can be particularly useful in assessing the specificity of the cognate TCR:MHC antigen interactions. Use of other internal controls of the invention is advantageous in detecting test compounds that specifically modulate interactions between the TCR and the MHC antigen molecule.

As will be more fully explained below, the present methods can accommodate a wide variety of TCR and MHC antigen molecules. Generally stated, suitable molecules can be naturally-occurring or recombinant and can usefully encompass a range of fully soluble or cell-anchored (membrane-bound) molecules. It is emphasized that this feature of the invention is very important as it provides, e.g., a framework for the design and implementation of many different and useful screening formats. This feature helps to expand opportunities for detecting test compounds with important therapeutic activity. Illustrative of such formats include protein-based and cell-based screening assays such as those already mentioned as well as those described below.

By the term “TCR molecule” or related phrase including the plural form as that term is used to describe a molecule used in a screening method of this invention is meant a naturally-occurring or recombinant molecule that includes at least a portion of the TCR V-α and V-β chains or the TCR V-γ and V-δ chains up to and including the entire length of each chain. Preferred chain lengths are sufficient to form a pocket or cleft that can specifically bind to superantigen or more preferably an antigen in the context of an MHC molecule or to an alloreactive MHC molecule. In most instances, the TCR molecule will bind MHC antigen molecule through a plurality of non-covalent bonds. Suitable TCR molecules can be mono- or multi-valent as needed in a particular assay format. Methods for identifying such TCR molecules are known and include standard TCR binding tests such as those described below.

Illustrative TCR molecules for use with this invention have been disclosed in pending U.S. patent application Ser. No. 08/943,006 entitled Soluble Single-chain T cell Receptors filed on Oct. 2, 1997 and Ser. No. 08/813,731 entitled Fusion Proteins Comprising Bacteriophage Coat Protein and a Single-chain T cell Receptor filed on Mar. 7, 1997; the disclosures of which are incorporated herein by reference.

The U.S. Ser. Nos. 08/943,006 and 08/813,731 applications each describe a wide variety of TCR molecules and functional fragments thereof suitable for use with the present methods. For example, a suitable sc-TCR molecule includes V-α and V-β chains covalently linked through a suitable peptide linker sequence. Particularly, the V-α chain can be covalently linked to the V-β chain through a suitable peptide linker sequence fused to the C-terminus of the V-α chain and the N-terminus of the V-β chain. The V-α and V-α chains of the sc-TCR fusion protein are generally about 200 to 400 amino acids in length, preferably about 300 to 350 amino acids in length, and will be at least 90% identical, and preferably 100% identical to the V-α and V-β chain s of a naturally-occurring TCR. By the term “identical” is meant that the amino acids of the V-α or V-β chain are 100% homologous to the corresponding naturally-occurring TCR V-β or V-α chain s.

As disclosed in the U.S. Ser. Nos. 08/943,006 and 08/813,731 applications, the V-β chain of the sc-TCR molecule can, if desired, further include a C-β chain or fragment thereof fused to the C-terminus of the V-β chain. Further, the V-α chain can include a C-α chain or fragment thereof fused to the C-terminus of the V-α chain and the N-terminus of the peptide linker sequence. Generally, in those fusion proteins including a C-β chain fragment, the fragment will have a length of approximately 50 to 126 amino acids and will usually not include the last cysteine residue at position 127. For those fusion proteins comprising a C-α chain, the length can vary between approximately 1 to 90 amino acids (i.e. the C-α chain up to but not including the final cysteine). For example, in one embodiment, the fusion protein includes a C-α chain fragment between about 1 to 72 amino acids starting from amino acid 1 to 72. In another embodiment, the C-α chain fragment is between about 1 to 22 amino acids starting from the first amino acid to 22 (leucine). The C-α chain fragment typically does not include any cysteine resides except the C_{∝90} variant which includes two cysteine residues.

As disclosed in the U.S. Ser. Nos. 08/943,006 and 08/813,731 applications, to facilitate expression of fully soluble and functional protein, the Ig-C_{L} chain or suitable Ig-C_{L} fragment is covalently linked to the sc-TCR molecule, e.g., to the C-terminus of the V-β chain or C-β fragment. Although typically not preferred, it is possible to covalently link the Ig-C_{L} or fragment thereof to the N-terminus of the V-α chain. In most cases, choice of Cα and Cβ chain length will be guided by several parameters including the particular V chains selected and intended use of the soluble fusion molecule.

Additional sc-TCR proteins of the invention include, but are not limited to those having two peptide linker sequences, where the first peptide linker sequence is fused between the C-terminus of the V-α chain and the N-terminus of the V-β chain. The C-terminus of the V-β chain can be fused to the N-terminus of a C-β chain fragment. The second peptide linker is then fused to the C-terminus of the V-β chain or C-β chain fragment and the N-terminus e.g., of an Ig-C_{L} chain, or Ig-C_{L} chain fragment or an effector or tag molecule.

In other illustrative embodiments, sc-TCR proteins can be made by fusing the V-β chain to the V-α chain through a suitable peptide linker in which the C-terminus of the V-β chain or C-β chain fragment thereof and the N-terminus of the V-α chain are covalently linked. An Ig-C_{L} chain or fragment can be covalently linked to the C- or N-terminus of the molecule as desired although generally linkage at the C-terminus will be preferred.

The peptide linker sequence of the sc-TCR proteins are typically selected so that the sc-TCR molecule forms a binding site which resembles that of a naturally-occurring TCR V-α and V-β chain. The Vα and Vβ chains can be derived from uninduced T-cells although in most cases the V chains are those associated with a pathology, e.g., an immune-related disorder or disease.

Specifically preferred linker sequences have been disclosed throughout the U.S. Ser. Nos. 08/943,006 and 08/813,731 applications.

As an illustration, the peptide linker sequence separating the Vα,β chains flexibly positions the V-chains in a pocket that is capable of specifically binding a ligand such as a desired MHC antigen. For example, ligand binding to the sc-TCR fusion protein can be used to modulate T-cell activity as determined by assays described in the U.S. Ser. Nos. 08/943,006 and 08/813,731 applications referenced above. Fusion of Ig-C_{L} chain or Ig-C_{L} chain fragment to the sc-TCR molecule can increase assay performance in some settings, e.g., by facilitating soluble expression of the sc-TCRs and maintaining sc-TCR integrity in aqueous solutions such as cell media.

As discussed above and in the U.S. Ser. Nos. 08/943,006 and 08/813,731 applications, an sc-TCR fusion protein of the invention can include, if desired, a covalently linked Ig-C_{L} chain or suitable Ig-C_{L} chain fragment linked, e.g., to the C-terminus of the sc-TCR molecule. In one embodiment, the sc-TCR fusion protein includes a fused mammalian Ig-C_{L} chain, preferably a full-length murine or human Ig-C_{L} chain such as the Cκ chain. The nucleic acid and protein sequences of several Ig-C_{L} chains have been disclosed. See, e.g., Fundamental Immunology, (1993)₃rd Ed. W. Paul. Ed. Rsen Press Ltd. New York; and Kabat, E. A., et al., (1991) Sequences of Proteins of Immunological Interest (5^{th} Ed.) Public Health Services, National Institutes of Health.

The term Ig-C_{L} chain is also meant to include an immunoglobulin light chain constant region which varies from a disclosed full-length sequence by one or more amino acid substitutions or additions. For example, an amino acid can be added to a disclosed Ig-C_{L} chain sequence at one or both ends of the chain, e.g., by conventional recombinant methods. In addition, the recombinant methods can be used to substitute a given amino acid in the chain if desired. Generally, the amino acid addition will include between about 1 to 30 neutral or hydrophilic amino acids, preferably between about 1 to 10 of such amino acids. An amino acid substituted for another amino acid in the Ig-C_{L} chain will be a conservative or non-conservative amino acid replacement. Accordingly, a tyrosine amino acid in a Ig-C_{L} chain sequence substituted with a phenylalanine will be an example of a conservative amino acid substitution, whereas an arginine replaced with an alanine would represent a non-conservative amino acid substitution. As will be pointed out below for Ig-C_{L} chain fragments, a sc-TCR fusion protein comprising the fused Ig-C_{L} chain will be fully soluble and functional.

In some embodiments of the present methods, it will be useful to use an Ig-C_{L} chain fragment such as a murine or human Cκ chain fragment. For example, a suitable Ig-C_{L} chain fragment can be fused to a sc-TCR molecule when it is desirable to minimize molecular weight of the fusion protein. By the phrase “suitable Ig-C_{L} chain fragment” or related term is meant an Ig-C_{L} fragment which when fused to a desired sc-TCR molecule forms a fully soluble and functional sc-TCR fusion protein as defined below. A desired Ig-C_{L} chain fragment (both Cκ and Cλ type) can be made according to standard recombinant methods, e.g., by PCR amplification of a murine or human Cκ or Cλ chain fragment followed by ligation of the PCR product to the DNA segment or vector encoding a desired sc-TCR molecule. As noted below, the PCR product can be manipulated to include restriction enzyme cleavage sites to facilitate cloning. Generally, a suitable murine or human Cκ chain fragment will be between about 70 to 150, preferably between about 90 to 120, and more preferably between about 100 to 110 amino acids in length. Examples of suitable murine or human CK chain DNA sequences are disclosed below. See Examples 5, 6 and 7 below.

Preferred sc-TCR proteins disclosed in the U.S. Ser. Nos. 08/943,006 and 08/813,731 applications are fully functional and soluble. By the term “fully functional” or similar term is meant that the fusion protein specifically binds ligand and particularly MHC antigen. Assays for detecting such specific binding are disclosed herein and include standard immunoblot techniques such as Western blotting.

The term “specific binding” or similar term is used herein to describe binding between an antibody and antigen. The antibody (preferably a monoclonal antibody) binds the antigen to form a specific binding pair. Typically, the antibody does not recognize and bind to other molecules as determined by Western blotting, ELISA, RIA, gel mobility shift assay, enzyme immunoassay, competitive assays, saturation assays or other suitable protein binding assays.

The term “specific immune complex” or similar term is used herein to reference a binding pair that includes a specific TCR molecule and its cognate MHC antigen molecule. The specific immune complex may include one type of antigen molecule or more than one type provided that each antigen molecule facilitates formation of the specific binding pair (ie. TCR:antigen:MHC molecule).

Methods for detecting specific immune complexes are known and will vary depending on intended use of the invention. For example, in embodiments in which screening is performed in an essentially cell free format (ie. protein-protein based), preferred methods will include Western blotting, ELISA, RIA, gel mobility shift assay, enzyme immunoassay, competitive assays, saturation assays or other suitable protein binding assays. In cell based embodiments of the invention including those screens in which one or more cell responses are used to identify candidate compounds, detection of those responses will be taken as being indicative of formation of the specific immune complexes. Examples of suitable cell responses are provided below and include cytokine production particularly from T cells.

The sc-TCR fusion proteins disclosed in the U.S. Ser. Nos. 08/943,006 and 08/813,731 applications can comprise Vα,β chains for which a substantially full-length coding sequence is readily available. Methods for obtaining full-length TCR V chain sequences from cell sources are well known. Alternatively, the Vα,β chains regions can be obtained by PCR amplification of publicly available Vα, β chains for which at least a portion of the sequence is known. Exemplary Vβ gene sequences include V β 8.1, V β 6.1, V β5.1, V β5.2, V β5.3, Vβ2.1, and V β 2.3 gene sequences. See Abe et al. (1992) PNAS (USA) 89: 4066; Wang, et al., 1993); PNAS (USA) 90: 188; Lahesma et al. (1993) J Immunol. 150: 4125; Kotzin, et al., (1991) PNAS (USA) 88: 9161; Uematsu, et al. (1991) PNAS (USA) 88: 8534. See also, Kabat, E. A., et al. supra and Chotia, C. et al., (1988) EMBO J. 7:3745 for additional TCR V chain sequence.

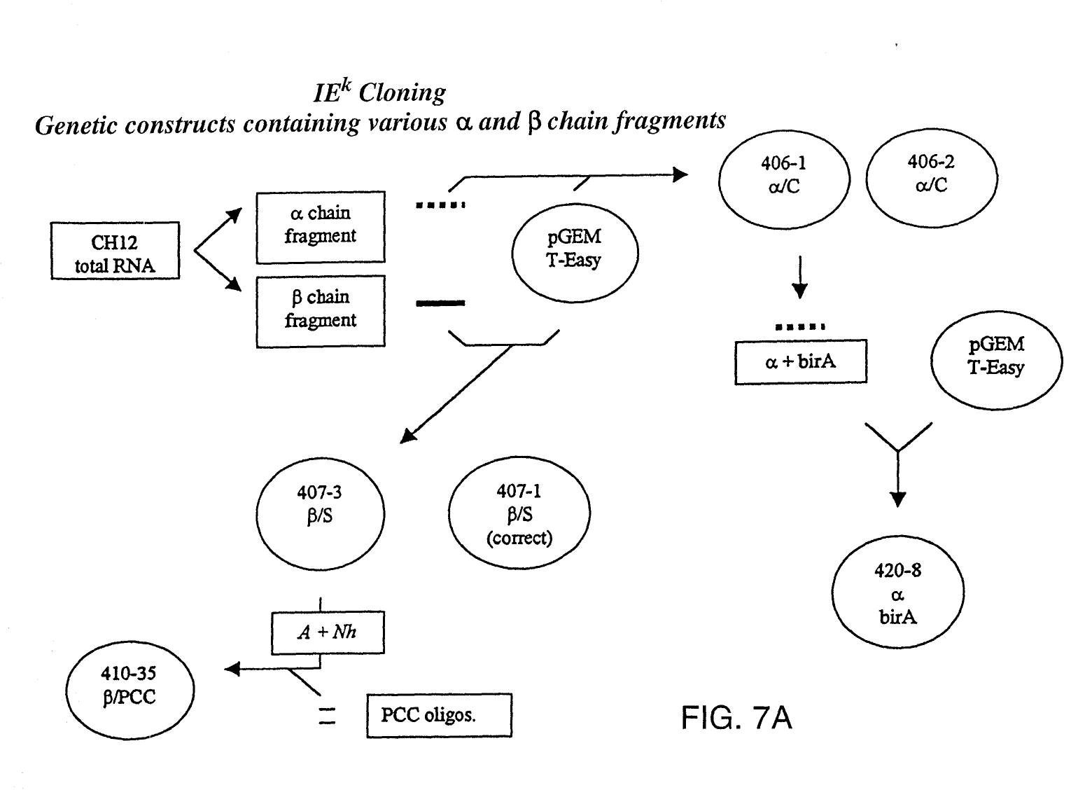

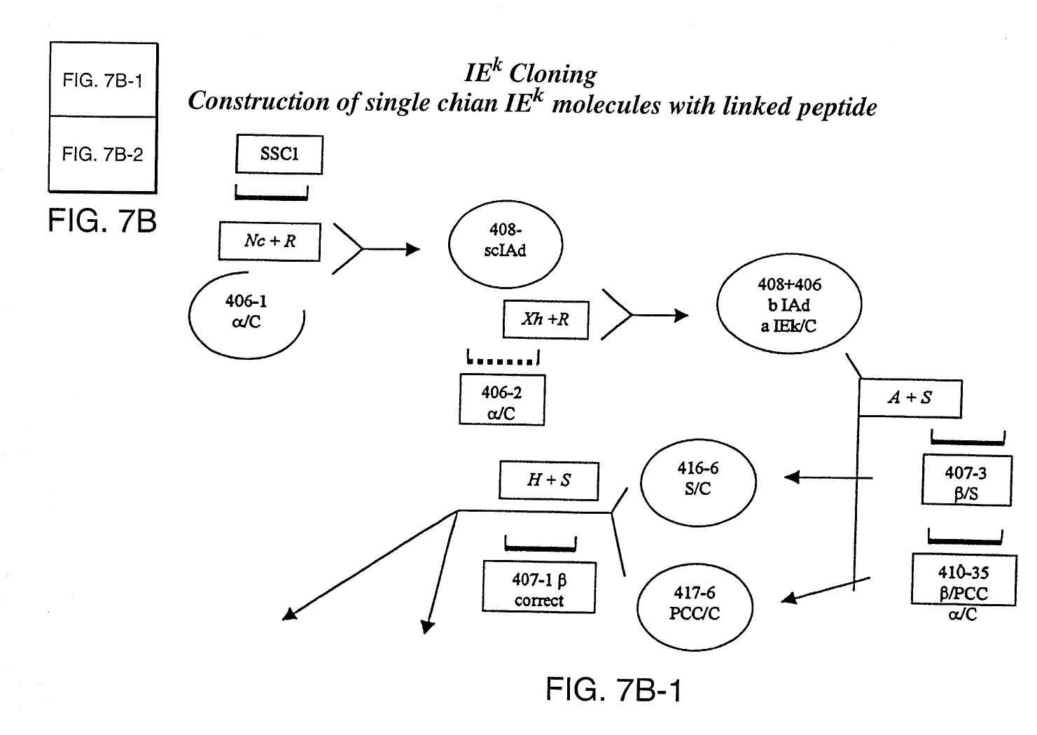

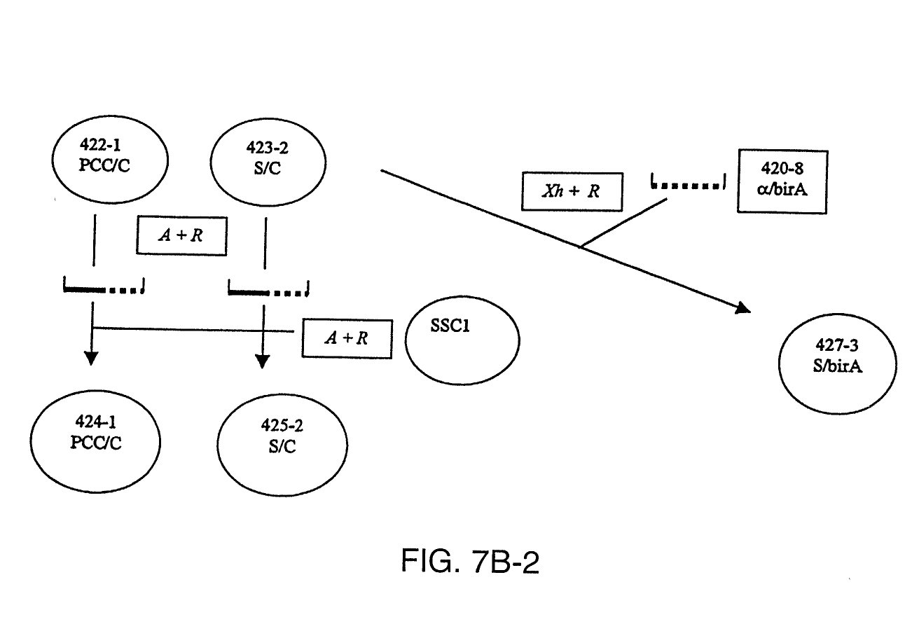

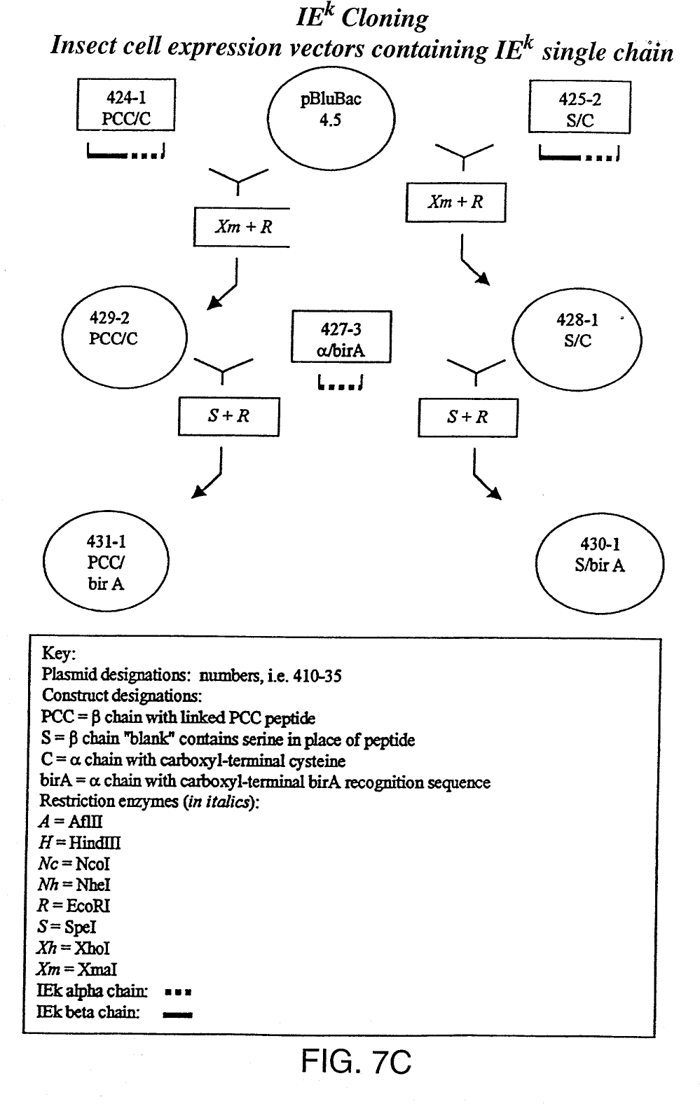

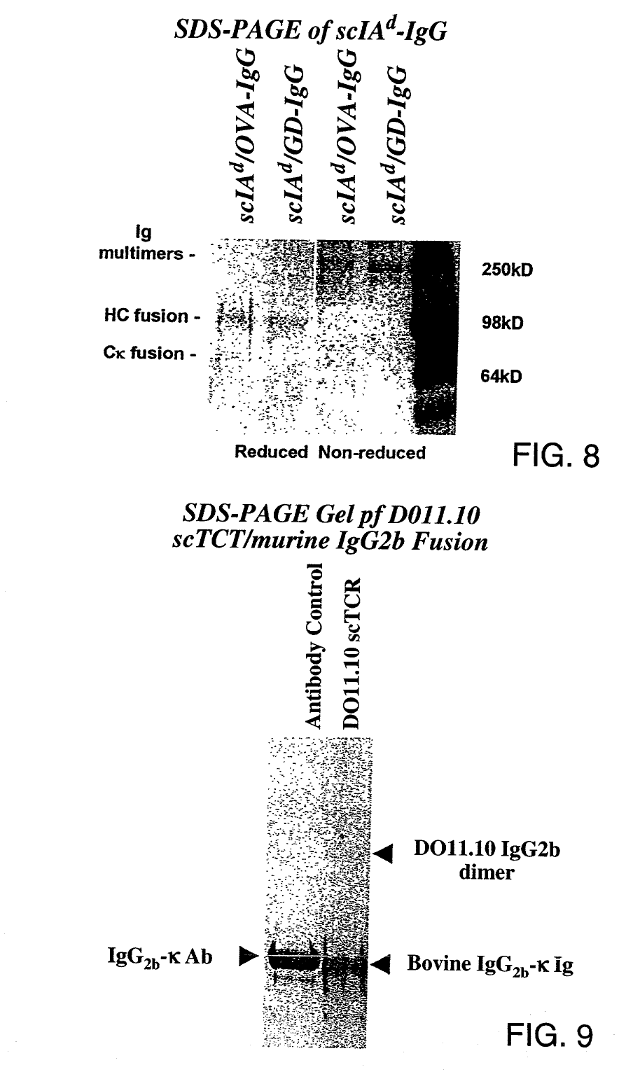

In particular, Example 3 and FIGS. 7 and 8 from the U.S. Ser. No. 08/943,066 application provides oligonucleotide primers for PCR amplifying a variety of V-α and V-β chain s.

As particularly disclosed in the U.S. Ser. No. 08/943,066 application, exemplary sc-TCR fusion proteins are generally encoded by a DNA segment including covalently linked in sequence: promoter/leader sequence/V-α chain/single-chain linker sequence/V-β chain/Cκ chain; promoter/leader sequence/V-α chain/single-chain linker sequence/V-β chain, C-β chain fragment/Cκ chain; promoter/leader sequence/V-α chain, C-α chain/single chain linker sequence/V-β chain/CK chain; or promoter/leader sequence/V-α chain, C-α chain fragment/single-chain linker sequence/V-β chain, C-β chain fragment/Cκ chain. Exemplary sc-TCR molecules are as described above except that an the Cκ chain is not encoded by the DNA segment. The DNA vectors encoding the sc-TCR proteins are introduced into desired cells, including those specific expression systems disclosed herein, for soluble expression of the fusion protein.

More specific vectors for making and using the scTCR molecules are taught throughout the U.S. Ser. Nos. 08/943,006 and 08/813,731 applications.

The methods of this invention are compatible with use of other TCR molecules including those in a heterodimeric format. Examples of such molecules include TCR V chains covalently linked to immunoglobulin constant regions. See e.g., Gregoire, C., et al. (1991) PNAS, 88, 8077 (reporting how to make and use heterodimeric αβ3 TCR chains that include a Cα,Vα sequence joined to the C region of a kappa light chain (Cκ)). The reference also disclosed a VβCβCκ sequence. See also Weber, S., et al. (1992) Nature, 356: 793.

See also the following references for examples of how to make and use other scTCR molecules for use with this invention. Novotny, J. et al. PNAS (USA) 88, 8646 (1991); Soo Hoo, W. F. et al. PNAS (USA) 89, 4759 (1992); Wülfing, C. and Plückthun, A., J. Mol. Biol. 242, 655 (1994); Kurucz, I. et al. PNAS (USA) 90 3830 (1993); PCT WO 96/13593; Ward, E. S. et al., J. Mol. Biol. 224, 885, (1992); Schlueter, C. J. et al. J. Mol. Biol. 256, 859 (1996); Mariuzza, R. A. and Winter, G., (1989) 264:7310; Gascoigne, N. R. J., et al., PNAS (USA) (1987), 84:2936.

Preferred TCR molecules for use in accord with of the invention are of sufficient size to allow for specifically binding of the TCR to the MHC antigen. In embodiments where the MHC antigen is a peptide-MHC molecule, the TCR molecules contain at least the CDR binding loops forming the MHC-peptide binding pocket. Useful α/β TCR molecules containing an MHC-peptide binding pocket preferably consists of at least the α chain variable domain (about amino acid 1 to about amino acid 110 to about 130 dependent on CDR length of the α chain) and β chain variable domain (about amino acid 1 to about amino acid 110 to about 130 dependent on CDR length of the β chain).

A more preferred TCR molecule for use in accord with this invention exhibits significant binding activity in what has been referred to as the standard TCR binding test. Preferably, the TCR molecule specifically binds its cognate MHC antigen molecule to a level that is at least about 50%, preferably at least about 80%, and more preferably at least about 90% to 99% as much as a suitable control. Examples of a suitable control include the TCR molecule as it occurs naturally (i.e., as a wild-type heterodimer) or a recombinant TCR. In most instances, the control will be a fully soluble recombinant TCR, preferably a soluble single-chain TCR.

In such an assay, TCR interaction with immobilized MHC antigen is monitored by surface plasmon resonance (SPR) with a BIAcore 2000 system (BIAcore, Inc.). Typically, about 500-5000 resonance units (RU) of MHC antigen are coupled to a BIAcore CM5 chip by standard NHS/EDC coupling chemistry. The subject TCR and control TCR (in PBS) are separately injected at a flow rate of 10-50 μl/min over the MHC antigen surface. Multiple injections are carried out in which the concentration of the each TCR range between 2 μM to 20 μM. Based on the SPR binding curves, rate constant analyses are performed using the BIAevaluation 2.1 software (BIAcore, Inc.). The calculated association and dissociation rate constants, affinity constant and half-life of the control TCR:MHC antigen interactions are taken as 100%.

By the term “MHC antigen” or related phrase including the plural form used in a screening method of this invention is used in reference to an MHC-based molecule that specifically binds the TCR. The MHC antigen can consist of but is not limited to MHC/peptide complexes, MHC/superantigen complexes, MHC/lipid (or glycolipid) complexes or alloreactive (xenoreactive) MHC molecules. The MHC component of the MHC antigen can consist of but is not limited to classical or nonclassical MHC class Ia, class Ib or class II molecules or to MHC homologues (including human HLA molecules) (See Maenaka, K & Jones, E. Y. 1999. Curr. Opin. Struct. Biol. 9:745-753). The MHC component used in a screening method of this invention is meant a naturally-occurring or recombinant molecule that includes at least a portion of the MHC chains up to and including the entire length of each chain. In one embodiment, these chains are sufficient to form a peptide (or lipid) binding groove or cleft that can specifically bind a presenting peptide (or lipid). In accord with this invention, the presenting peptide can be non-covalently bound to the peptide binding groove of an “empty” MHC component via stable hydrogen bonding. Such complexes are often referred to as being “loaded”. Also in accord with this invention, the MHC antigen molecule can include a covalently linked (i.e. fused) presenting peptide such as those class I and class II molecules disclosed in U.S. Pat. No. 5,869,270; the disclosure of which is incorporated herein by reference. Suitable MHC antigen molecules for use with this invention can be mono- or multi-valent as described below. In embodiments in which the MHC component is a naturally occurring or recombinant class I molecule, the MHC antigen molecule may further include a naturally occurring or recombinant β2-microglobulin molecule.

In another embodiment, the MHC antigen molecule can consist of naturally-occurring or recombinant molecules that includes at least a portion of the MHC chains and superantigen chain up to and including the entire length of each chain. These chains are sufficient to form a MHC/superantigen complex. Superantigens are proteins that are capable of binding to TCRs utilizing particular V gene segments, regardless of the nature of the other parts of the TCR. See Kotzin, B. L. et al (1993) Adv. Immunol. 54:99-166. Preferred superantigens of the invention include Mls antigens, SEA, SEB, SEC 1, SEC2, SEC3, SED, SEE, SPE-A, SPE-B, SPEC, ExFT and TSST.

In another embodiment, the MHC antigen molecule can consist of naturally-occurring or recombinant molecules that includes at least a portion of an alloreactive (xenoreactive) MHC chains up to and including the entire length of each chain. These chains are sufficient to form an alloreactive MHC complex. Alloreactive MHC molecules are non-self MHC molecules recognized by (self) T cells as foreign molecules. Binding of the TCR to the alloreactive MHC molecule may or may not be dependent on particular peptide antigens bound to the MHC molecule. See Obst, R. et al. (2000) J. Exp. Med 191:805-812.

Class I molecules are integral membrane proteins comprising a glycoprotein heavy chain having three extracellular domains (i.e. α1, α2 and α3), a transmembrane domain and a cytoplasmic domain. The heavy chain is non-covalently associated with a soluble subunit β2-microglobulin. The α1 and α2 domains of the heavy chain fold together to form the peptide binding groove. The association between the heavy chain and β2-microglobulin helps to stabilize the peptide binding groove. In embodiments in which the MHC component is a class I molecule, the MHC component may consist of any combination of a naturally occurring or recombinant class I heavy chain (or fragments thereof) and a naturally occurring or recombinant β2-microglobulin molecule (or fragments thereof). In other embodiments, the MHC component can consist of a single chain construct of the class I heavy chain (or fragment thereof) and β2-microglobulin (or fragments thereof). The MHC component of the invention can consist of any fragment (in any molecular arrangement) of the class I heavy chain and/or β2-microglobulin sufficient to allow for formation of a functional MHC antigen (i.e. an MHC antigen capable of specific interaction with the TCR). The MHC component of the invention can also consist of a chimeric molecule comprised of any fragment (in any molecular arrangement) of the class II chains, class I heavy chain and/or β2-microglobulin sufficient to allow for formation of a functional MHC antigen.

Additional MHC antigen molecules suitable for use with the present invention have been disclosed in U.S. patent application Ser. No. 08/776,084 entitled MHC Complexes and Uses Thereof, filed on Jan. 17, 1997, which application is based on PCT application PCT/US95/09816 and is a continuation of U.S. patent application Ser. No. 08/382,454, filed on Feb. 1, 1995, which application is a continuation of U.S. patent application Ser. No. 08/283,302, filed on Jul. 29, 1994; the disclosures of which are incorporated herein by reference.

Particularly disclosed in the Ser. No. 08/776,084, PCT/US95/09816, Ser. Nos. 08/382,454, and 08/283,302 patent applications are a wide variety of heterodimeric MHC molecules (class I and class II) suitable for use with this invention. See also McCluskey, J. et al. J. Immunol. 141: 1451; PCT/US92/10030; and U.S. Pat. No. 5,820,866 to Kappler and Marrack (disclosing additional class I and class II MHC molecules); the disclosures of which are incorporated herein by reference.

Additionally suitable MHC antigen molecules have been disclosed in U.S. patent application Ser. No. 08/960,190 entitled Soluble MHC Complexes and Methods of Use Thereof, filed on Oct. 29, 1997, PCT WO 96/04314 and PCT 97/28191; the disclosures of which are hereby incorporated by reference.

Briefly, the applications disclose highly useful single-chain (“se-”) MHC class I and class II complexes suitable for use with this invention. Particularly disclosed are sc-MHC class I and class II complexes with recombinantly fused presenting peptides (referred to as sc-MHC peptide fusion molecules), empty sc-MHC molecules (referred to as having no recombinantly fused presenting peptides), and loaded sc-MHC complexes (referred to as including non-covalently attached presenting peptides).

More particularly disclosed in the Ser. No. 08/960,190, PCT WO 96/04314 and PCT 97/28191 applications is how to make and use a variety of sc-MHC molecules with a modified class II β2 chain and/or a fused Ig-C_{L} chain or suitable Ig-C_{L} chain fragment as provided. Also reported are what is referred to as “polyspecific” MHC complexes that have or do not have the modified class II β2 chain and/or the fused Ig-C_{L} chain or Ig-C_{L} chain fragment.

See also the U.S. Pat. No. 5,869,270 to Rhode, P. et al. (disclosing additionally preferred sc-MHC molecules for use with this invention); the disclosure of which has already been incorporated herein by reference.

Particular MHC antigen molecules for use in accordance with the invention contain an MHC component of sufficient size to allow for specifically binding of the MHC antigen to the TCR. In embodiments where the MHC antigen is a peptide-MHC molecule, the MHC component contains at least a peptide binding groove. Useful class II MHC components containing a peptide binding groove consists of at least the class II α1 domain (about amino acid 1 to about amino acid 84 of the α chain) and β1 domain (about amino acid 1 to about amino acid 95 of the β chain). Useful class I MHC components containing a peptide binding groove consist of at least the class I α1 and α2 domains (about amino acid 1 to about amino acid 182 of the heavy chain). In embodiments where the MHC antigen is an alloreactive MHC molecule, the MHC component contains at least the alloreactive MHC determinant and, in the case where alloreactivity is dependent on peptide, a peptide binding groove. The nature of the alloreactive MHC determinant and dependency on peptide vary depending on the TCR specificity and can be determined experimentally. See Obst, R. et al. (2000) J. Exp. Med. 191:805-812. In embodiments where the MHC antigen is a superantigen-MHC complex, the MHC component contains at least the superantigen binding site. The superantigen binding site on the MHC component lies outside of the peptide-binding groove and varies dependent on the superantigen component. See Kotzin, B. L. et al (1993) Adv. Immunol. 54:99-166. Methods for identifying suitable MHC antigen molecules include standard MHC antigen binding tests described below.

A more preferred MHC antigen molecule for use in accord with this invention exhibits significant binding activity in what has been referred to as the standard MHC antigen binding test. Preferably, the MHC antigen molecule specifically binds its cognate TCR molecule to a level that is at least about 70%, preferably at least about 80%, and more preferably at least about 90% to 99% as much as a suitable control. Examples of a suitable control include the MHC antigen molecule as it occurs naturally (i.e., as a wild-type heterodimer) or a recombinant MHC antigen molecule. In most instances, the control will be a fully soluble recombinant MHC antigen molecules, preferably a soluble single-chain MHC antigen molecule. Methods for making and using such molecules have been fully described above including the U.S. Pat. No. 5,869,270.

In such an assay, MHC antigen interaction with immobilized TCR is monitored by surface plasmon resonance (SPR) with a BIAcore 2000 system (BIAcore, Inc.). Typically, about 500-5000 resonance units (RU) of TCR are coupled to a BIAcore CM5 chip by standard NHS/EDC coupling chemistry. The subject MHC antigen and control MHC antigen are separately injected in PBS at a flow rate of 10-50 μl/min over the TCR surface. Multiple injections are carried out in which the concentration of the each MHC antigen range between 2 μM to 20 μM. Based on the SPR binding curves, rate constant analyses are performed using the BIAevaluation 2.1 software (BIAcore, Inc.). The calculated association and dissociation rate constants, affinity constant and half-life of the control TCR:MHC antigen interactions are taken as 100%.

As discussed, in certain invention embodiments it will be useful to use MHC molecules that include lipid, glycolipid, alloreactive, xenoreactive and/or superantigen components.

In embodiments in which MHC/lipid and glycolipid molecules are of interest, there is an MHC class I-like family of molecules that can be used with this invention. Preferred members of that family are known as the CD 1 family. There is recognition that this specific family can present bacterial lipid antigens to T cells. Specific examples of these complexes include CD1c/hexosyl-1-phosphoisoprenoid, CD1c/mannosyl-beta1-phosphodolichol, CD1b/lipoarabinomannan, CD1b/mycolic acid lipid. See generally Moody D B, et al. (1999) Immunol Rev. 172:285-96 as well as references disclosed therein.

Particular alloreactive MHC molecules for use in accord with this invention can include nearly any human MHC molecule present in an organ or tissue graft. Preferably, that molecule is not shared by the host (leading to host-versus-graft T cell responses) or any MHC molecules present in the host that are not shared by the graft (leading to graft-versus-host T cell responses).

Generally, the graft survival is most dependent on lowering the alloreactive response to MHC class II antigens. More specific alloreactive MHC molecules include those predominant in the Caucasian population, e.g., HLA-A1, HLA-A2, HLA-B7, HLA-B35, HLA-B44, HLA-DR1, HLA-DR2, HLA-DR3, HLA-DR4, HLA-DR5, HLA-DR6, HLA-DR7, HLA-DQ6, HLA-DQ7 and HLA-DQ8. See generally Cellular and Molecular Immunology 3rd Ed. Abbas, Lichtman, Pober, Abbas and Schmitt, W. B. Saunders, Philadelphia, 1997.

In addition, it is possible to use many xenoreactive MHC molecules with this invention including xenograft MHC molecules. More specific examples include swine MHC molecules in swine xenograft organ transplants, human MHC molecules reactive to xenograft T cells. Preferred human MHC molecules include those listed above. Preferred swine MHC molecules include SLA-1 to −6, SLA-DR, and SLA-DQ molecules. See generally Chardon P, et al. (1999) Immunol Rev. 167:179-92 and references disclosed therein.

Additionally useful TCR molecules and MHC antigens molecules for use with the present invention are disclosed in the Examples Section, below.

In another aspect, the present invention provides suitable test compounds detected and identified by the methods provided herein.

As discussed, practice of the invention now makes it possible to detect test compounds that modulate interactions between the TCR and MHC antigen molecule in a protein: protein, protein:cell or cell:cell assay format. As also discussed, relevant TCR and MHC antigen interactions, generally detected through the resulting immune complex, can be measured directly or indirectly as needed.

Particular methods of this invention are designed to detect and identify test compounds that interact with (e.g., bind to) a TCR molecule. The term “interact” is meant to include, but is not limited to the extracellular domain (ECD-TCR) or the cytoplasmic domain (CD-TCR) of the TCR molecule. Preferred interactions will occur at or near the binding site, pocket or cleft responsible for specific binding to a cognate MHC antigen molecule. Thus test compounds of special interest will interact with the TCR pocket or cleft that can bind to superantigen or peptide antigen in the context of a suitable MHC molecule or to an alloreactive MHC molecule.

Additional methods are also designed to identify compounds that interact with (e.g., bind to) an MHC antigen molecule. Thus the term “interact” in the context of the MHC antigen molecule includes, but is not limited to, the extracellular domain (ECD-MHC) or the cytoplasmic domain (CD-MHC) of the MHC molecule, the peptide antigen or the superantigen. Preferred interactions will occur at or near the MHC peptide (or lipid) binding groove and other sites impacting specific binding with the TCR molecule. More particular compounds will often interact with the MHC peptide binding groove or near that groove.

Additional methods are also designed to identify compounds that interact with (e.g., bind to) an TCR:MHC antigen complex. Thus the term “interact” in the context of the TCR:MHC antigen complex includes, but is not limited to, the ECD-TCR, the CD-TCR, the ECD-MHC, the CD-MHC, the peptide antigen or the superantigen. Preferred interactions will occur at or near sites impacting the stability of the complex or at or near sites impacting the ability of the complex to form multimers.

Suitable test compounds for use in accord with the screening methods of this invention include, but are not limited to, peptides, polypeptides, antibodies and fragments thereof, and other organic compounds (e.g., peptidomimetics and non-peptidomimetics) that bind to the TCR molecule, the MHC antigen molecule, or both; and either mimic the activity triggered by specific binding between the TCR and MHC antigen molecule (i.e., an agonistic compound) or inhibit the activity triggered (i.e., an antagonistic compound); as well as peptides, antibodies or fragments thereof, and other organic or in organic compounds that can increase or decrease specific binding between a desired TCR molecule and its cognate MHC antigen molecule.

Such compounds can particularly include, but are not limited to, peptides such as, for example, soluble peptides, including but not limited to members of random peptide libraries; (see, e.g., Lam, K. S. et al., 1991, Nature 354:82-84; Houghten, R. et al., 1991, Nature 354:84-86), and combinatorial chemistry-derived molecular library made of D- and/or L-configuration amino acids, phosphopeptides (including, but not limited to, members of random or partially degenerate, directed phosphopeptide libraries; see, e.g., Songyang, Z. et al., 1993, Cell 72:767-778). Also contemplated are compounds that include or consist of antibodies (including, but not limited to, polyclonal, monoclonal, humanized, anti-idiotypic, chimeric or single chain antibodies, and Fab, F(ab′)₂ and Fab expression library fragments, and epitope-binding fragments thereof), as well as small, medium and large organic or inorganic molecules. See U.S. Pat. No. 5,980,892, for example.

For more specific disclosure relating to making and using a variety of molecular libraries suitable for use with this invention see e.g., U.S. Pat. No. 6,001,579 (tag encoded combinatorial libraries); U.S. Pat. No. 5,573,905 (encoded combinatorial libraries); U.S. Pat. No. 5,639,603 (diverse molecular libraries); U.S. Pat. No. 5,880,972 (methods and apparatus for making combinatorial chemistry libraries); the disclosures of which are incorporated herein by reference. See also Nielsen, J. et al. (1993) J. Am. Chem. Soc. 115: 9812; Brenner, S. and R. Lerner (1992) PNAS (USA) 89: 5381-5383; and Pinella, C. et al. (1992) Biotechniques, 13: 901. See also EPO publication no. 0 432 691 A1 (disclosing certain peptides that bind MHC molecules).

Alternatively, or in addition, many useful chemical libraries can be obtained from commercial vendors such as Chembridge, ArQule, CombiChem, Pharmacopeia, Trega 2 Biopharmaceuticals, and Tripos.

However, in some instances a more focused screen of test compounds may be desired. In many of these cases, standard computer and searching technologies can permit pre-identification of candidate compounds, as well as manipulation of previously identified compounds. Such compounds can thus be “pre-screened” for capacity to modulate interaction between a given TCR and MHC antigen molecules. Such “pre-screening” of candidate compounds can be accomplished by performing the following general steps:

1. For example, having found such TCR and MHC antigen molecules that specifically interact to form an immune complex, the active sites or regions are identified. Such active sites might typically be ligand binding sites, such as the interaction domains of the TCR molecule, MHC antigen molecule; or both molecules. The active site can be identified using standard methods including, for example, from characterization of the amino acid sequences of the TCR or MHC antigen polypeptide chains or from study of complexes of the TCR with its cognate MHC antigen. In the former case, sequence homology comparison with known active sites on other TCR or MHC antigen molecules can be used to find the active site. In the latter case, mutational, chemical or X-ray crystallographic methods can be used to find the active site by finding where on the TCR or MHC antigen the cognate ligand is found.

2. Next, the three dimensional geometric structure of the active site is determined. This can be done by known methods, including X-ray crystallography, which can determine a nearly complete molecular structure at the atomic level. On the other hand, solid or liquid phase NMR can be used to determine certain intra-molecular distances. Any other experimental method of structure determination can be used to obtain partial or complete geometric structures. The geometric structures may be measured with a complexed ligand, natural or artificial, which may increase the accuracy of the active site structure determined.

If an incomplete or insufficiently accurate structure is determined, the methods of computer based numerical modeling can be used to complete the structure or improve its accuracy. Nearly any recognized modeling method may be used, including parameterized models specific to particular biopolymers such as proteins or nucleic acids, molecular dynamics models based on computing molecular motions, statistical mechanics models based on thermal ensembles, or combined models. For most types of models, standard molecular force fields, representing the forces between constituent atoms and groups, are often necessary, and can be selected from force fields known in physical chemistry. The incomplete or less accurate experimental structures can serve as constraints on the complete and more accurate structures computed by these modeling methods.

3. Finally, having determined the structure of the active site, either experimentally, by modeling, or by a combination, candidate modulating compounds can be identified by searching databases containing compounds along with information on their molecular structure.

Such a search seeks compounds having structures that match the determined active site structure and that interact with the groups defining the active site. Such a search can be manual, but is preferably computer assisted. Compounds found from this search can be viewed as those with potential to modulate TCR:MHC antigen interactions.

Alternatively, these methods can be used to identify improved modulating compounds from an already known modulating compound. The composition of the known compound can be modified and the structural effects of modification can be determined using the experimental and computer modeling methods described above applied to the new composition. The altered structure is then compared to the active site structure of the compound to determine if an improved fit or interaction results. In this manner systematic variations in composition, such as by varying side groups, (e.g., methyl, hydroxyl, carbonyl, carboxyl, hydrogen, and the like) can be quickly evaluated to obtain modified modulating compounds or ligands of improved specificity or activity.

Examples of molecular modeling systems include, but are not limited to, the CHARMM and QUANTA programs (Polygen Corporation, Waltham, Mass.). CHARMM performs the energy minimization and molecular dynamics functions. QUANTA performs the construction, graphic modeling and analysis of molecular structure. QUANTA allows interactive construction, modification, visualization, and analysis of the behavior of molecules with each other. See the following references for general disclosure relating to computer modeling of compounds such as drugs that are interactive with specific-proteins: Rotivinen, et al., 1988, Acta Pharmaceutical Fennica 97:159-166; Ripka, New Scientist 54-57 (Jun. 16, 1988); McKinaly and Rossmann, 1989, Annu. Rev. Pharmacol. Toxiciol. 29:111-122; Perry and Davies, OSAR: Quantitative Structure-Activity Relationships in Drug Design pp. 189-193 (Alan R. Liss, Inc. 1989); and Lewis and Dean, 1989 Proc. R. Soc. Lond. 236:125-140 and 141-162.

Other computer programs that screen and graphically depict chemicals are available from companies such as BioDesign, Inc. (Pasadena, Calif.), Allelix, Inc. (Mississauga, Ontario, Canada), and Hypercube, Inc. (Cambridge, Ontario). Although these are primarily designed for application to drugs specific to particular proteins, they could in principle be adapted to design drugs that modulate TCR and MHC antigen interactions. Such drugs could then be further screened in accordance with the methods of this invention.

Although the foregoing computer assisted methods can be used in some embodiments of this invention to “pre-screen” candidate compounds, it will often be more desirable to use the present methods with known libraries of candidate compounds. Alternatively, highly useful chemical libraries can be made and used according to established procedures. Such libraries may include, without limitation, natural products or synthetic chemicals, and biologically active materials, including amino acids (including modified amino acids), peptides, polypeptides, and proteins. Such libraries serve as a rich source of candidate compounds that can be selected to identify those acting as antagonists or agonists of interactions between the TCR and MHC antigen molecules. See generally Drews J. (2000) Science 287:1960-1964, Schreiber S. L. (2000) Science 287:1964-1969.

Optimal practice of the present invention usually involves use of an acceptable in vitro detection format which format, as discussed, can be direct or indirect as needed. A preferred detection format is designed to detect compounds that interact with (e.g., bind to) the subject TCR molecule, MHC antigen molecule; or both. Such detection formats usefully monitor and preferably quantify presence of the immune complex in the presence and absence of the test compound.

As mentioned, the invention is compatible with one or a combination of formats for detecting the immune complex. For example, presence of the immune complex (or even one of the control immune complexes) can be achieved according to established direct or indirect detection principles. Those skilled in the field of the invention will be able to determine operative and optimal assay conditions for particular combinations of TCR and MHC antigen molecules by virtue of the guidance provided herein and sometimes a modest level of routine experimentation.

With respect to particular formats, one way to detect presence of the immune complex is to detectably label an antibody capable specifically binding the complex. An illustrative label is linking the antibody, usually a monoclonal antibody, to an enzyme and using the detectably labeled antibody an enzyme immunoassay (EIA). See Voller, A., “The Enzyme Linked Immunosorbent Assay (ELISA)”, 1978, Diagnostic Horizons 2:1-7, Microbiological Associates Quarterly Publication, Walkersville, Md.;; Voller, A. et al., 1978, J. Clin. Pathol. 31:507-520; Butler, J. E., 1981, Meth. Enzymol. 73:482-523; Maggio, E. (ed.), 1980, Enzyme Immunoassay, CRC Press, Boca Raton, Fla.; Ishikawa, E. et al., (eds.), 1981, Enzyme Immunoassay, Kgaku Shoin, Tokyo).

See also Harlow and Lane (eds.) in: Antibodies: A Laboratory Manual 1988, Cold Spring Harbor Laboratory, New York; and Bishop, G. A. et al. (1992) in Biotechniques 12: 326-330 (disclosing an cellular ELISA for detection of cell surface material); and Kelly, T. A. et al. (1999) J. of Immunol. 5173-5177; the disclosures of which is incorporated herein by reference.

In this embodiment of the detection method, the enzyme which is bound to the antibody will react with an appropriate substrate, preferably a chromogenic substrate, in such a manner as to produce a chemical moiety that can be detected, for example, by spectrophotometric, fluorimetric or by visual means. Enzymes which can be used to detectably label the antibody include, but are not limited to, malate dehydrogenase, staphylococcal nuclease, delta-5-steroid isomerase, yeast alcohol dehydrogenase, alphaglycerophosphate, dehydrogenase, triose phosphate isomerase, horseradish peroxidase, alkaline phosphatase, asparaginase, glucose oxidase, beta-galactosidase, ribonuclease, urease, catalase, glucose-6-phosphate dehydrogenase, glucoamylase and acetylcholinesterase. The detection can be accomplished by colorimetric methods which employ a chromogenic substrate for the enzyme. Detection may also be accomplished by visual comparison of the extent of enzymatic reaction of a substrate in comparison with similarly prepared standards.

Good detection may also be achieved by using any of a variety of other immunoassays. For example, by radioactively labeling the antibodies or antibody fragments capable of specifically binding the immune complex, it is possible to detect TCR:MHC antigen interactions in the methods of this invention through the use of a radioimmunoassay (RIA) (see, for example, Weintraub, B., Principles of Radioimmunoassays, Seventh Training Course on Radioligand Assay Techniques, The Endocrine Society, March, 1986, which is incorporated by reference herein). The radioactive isotope can be detected by such means as the use of a gamma counter or a scintillation counter or by autoradiography.

Also, it is possible to label the antibody or fragment thereof with a fluorescent compound. When the fluorescently labeled antibody or fragment is exposed to light of the proper wave length, its presence can then be detected due to fluorescence. Among the most commonly used fluorescent labeling compounds are fluorescein isothiocyanate, rhodamine, phycoerythrin, phycocyanin, allophycocyanin, o-phthaldehyde and fluorescamine.

Further, the antibody or fragment can also be detectably labeled using fluorescence emitting metals such as ¹⁵²Eu, or others of the lanthamide series. These metals can be attached to the antibody using such metal chelating groups as diethylenetriaminepentacetic acid (DTPA) or ethylenediaminetetraacetic acid (EDTA).

For example, see the HTRF (homogeneous time-resolved fluorescence) antibody reagent for assay development and high throughput screening using europium provided by the Packard Instrument Company. See also U.S. Pat. No. 4,568,649; EPO 0154734 and Japanese patent application no. 84, 52452; the disclosures of which are incorporated herein by reference.

Other methods for detectably labeling the antibody or fragment are well known to those working in this field. For example, the antibody can be detectably labeled by coupling it to a chemiluminescent compound. The presence of the chemiluminescent-tagged antibody is then determined by detecting the presence of luminescence that arises during the course of a chemical reaction. Examples of particularly useful chemiluminescent labeling compounds are luminol, isoluminol, theromatic acridinium ester, imidazole, acridinium salt, adamantyl dioxetamer, thioxene derivatives and oxalate ester. Further, the antibody or fragment can also be detectably labeled using photosensitizer compounds. See e.g., U.S. Pat. No. 5,807,675 and references disclosed therein.

Also envisioned are detection formats based, in part or in whole, on biotinylation of molecules and using streptavidin or conjugates thereof to visualize the biotinylated molecules. More specific examples of this format are provided in the following Examples.

Likewise, a bioluminescent compound may be used to detectably label the antibody or fragment used to detect presence of the immune complex. Bioluminescence is a type of chemiluminescence found in biological systems in, which a catalytic protein increases the efficiency of the chemiluminescent reaction. The presence of a bioluminescent protein is determined by detecting the presence of luminescence. Important bioluminescent compounds for purposes of labeling are luciferin, luciferase and aequorin.

The antibody or fragment thereof can also be labeled with a protein that has fluorescent properties. Examples of such proteins include green fluorescent protein and its derivatives.

Further, a bioeffector tag may be used to detectably label the antibody or fragment used to detect presence of the immune complex. The bioeffector tag is a compound that is capable of effecting a measurable biological response, i.e. a cellular responses detected in vitro. Examples of useful bioeffector tags are natural and artificial ligands or antibodies (or fragments thereof) directed against cellular receptors. Particularly useful bioeffector tags are cytokines such as IL-2.

Examples of other suitable protein (and non-protein) tags are disclosed in the U.S. pat. appl. Ser. No. 08/813,781 filed on Mar. 7, 1997, as well as the U.S. Ser. No. 09/422,375, filed on Oct. 21, 1999; the disclosures of which are incorporated herein by reference. Particular examples of such probes include EE, myc, 6Xhis. Also preferred are tags that are specifically bound by a protein including an antibody, cell receptor, protein A, protein G, avidin, streptavidin; or a functional fragment thereof. Additionally useful tags include those which can be directly or indirectly modified by one or more proteolytic enzymes, protein kinase, biotin ligase; or a functional fragment thereof.

Alternatively, it may be useful in some embodiments to label the immune complex directly. This can be accomplished by a variety of methods including suitably labeling at least one of the TCR molecule, MHC antigen molecule, or the test compounds. When the molecule to be labeled is a protein, any of the methods described herein for labeling antibodies can be similarly to label TCR and/or MHC antigen molecules. Use of radionuclides (radioactive isotopes) such as those of iodine, e.g., ¹²⁵I or ¹³¹I may be indicated. In embodiments in which the test compounds are detectably labeled, choice of an appropriate radionuclide will depend, e.g., on the chemical properties of the test compound and the degree of sensitivity required. Such radionuclides can include, e.g., ³H, ¹⁴C, ³²P and the like.

A particular method of labeling MHC peptides has been disclosed in EPO 0 432 691 A1. Additional peptides have been disclosed. See Hemmer et al. (1998) J. of Immunol. 3631-3636 and U.S. Pat. No. 5,614,192 (disclosing therapeutic T cell receptor peptides).

Further specific detection formats are well-suited for use with the present invention.

More particular detection formats involve detecting presence of the immune complex indirectly, e.g., by monitoring and often quantifying at least one cell response. That cell response is pre-determined to be impacted by presence of the immune complex. Thus, in many embodiments of the invention, detection of the cell response in a method is taken to be indicative of presence of the immune complex. Examples of suitable cell responses include increases or decreases in at least one of cell adhesion, membrane potential, intracellular or extracellular ion concentration, intracellular kinase activity, phosphatase activity, intracellular protein transport, endogenous or heterologous gene expression, protein production or secretion including the production of at least one cytokine, cell proliferation, apoptosis, RNA synthesis, or DNA synthesis. See generally Cellular and Molecular Immunology 3d Ed. Abbas, Lichtman, Pober Abbas and Schmitt, W. B. Saunders, Philadelphia, 1997.

Of course in embodiments of the present invention in which a protein:protein screening method is employed it may be more useful to monitor directly or indirectly formation of the immune complex along lines previously discussed.

However in embodiments in which at least one of the TCR or MHC antigen molecules is manipulated in a cell based format it may, in some instances, be more useful to detect at least one of the cell responses as being indicative of the immune complex. In one embodiment, cells expressing the TCR molecule, e.g., T cells, T cell hybridomas or cells expressing recombinant TCRs, are monitored for manifestation of at least one of the cell responses.

By way of illustration, in embodiments in which a cell is expressing the TCR molecule at least one of the cell responses detected can be cell mediated synthesis and secretion of at least one cytokine, e.g., interleukin-2 (IL-2). In this example of the invention, production of the cytokine (or more than one cytokine), when compared with to one or more suitable controls is taken to be indicative of productive TCR:MHC antigen interactions and more specifically, presence of the immune complex. One or a combination of different strategies including methods discussed above can be used to detect presence of the cytokine in a particular assay format. In a more particular example, the cytokine is detected in an antibody “sandwich” type assay in which a first antibody, usually a monoclonal antibody, is coated on to a solid support where it specifically binds the cytokine and a second antibody, also usually a monoclonal antibody, binds the immobilized cytokine at a different site than the first antibody. In many embodiments, detectably labeling at least the second antibody will be generally preferred.

See Schwarz, M. K. et al. (1999) in Curr. Opin. in Chem. Biol. 3: 407-417 for a discussion relating to cytokines.

In additional embodiments, cell responses resulting from the formation of the TCR:MHC antigen immune complex may be detected as changes in gene expression. Changes in gene expression can be measured by a variety of methods, including measurement of transcription activity or messenger RNA levels of inducible or repressible genes. In addition, changes in promoter activity can be examined using endogenous or exogenous “reporter” genes. For example, a reporter construct can be generated in which a TCR responsive promoter element such as the IL-2 promoter is linked to the reporter gene for a detectable protein such as firefly luciferase or green fluorescent protein. The construct could then be stably introduced into a cell line expressing TCR on its surface. In these cells, induction of the reporter gene transcription and the resulting increase in luciferase activity, when compared with to one or more suitable controls, is taken to be indicative of productive TCR:MHC antigen interactions and more specifically, presence of the immune complex. Luciferase activity can be measured by standard methods for detecting bioluminescent following luciferase-dependant oxidation of the luciferin substrate. For example, assay reagents (Steady-GloI) specifically designed for measuring luciferase activity in high throughput screening formats are available from Promega Corp.

In yet additional embodiments, cell responses resulting from the formation of the TCR:MHC antigen immune complex may be detected as changes in intracellular ion concentrations. For example, formation of the TCR:MHC antigen complex results in increases in intracellular calcium ion concentration and acidification of the T cell. The level of these responses correlates with the ability of the MHC antigen to act as an agonist, partial agonist and antagonist (See Wulfing, C. et al. 1997. J. Exp. Med. 185:1815-1825). In this example of the invention, changes in intracellular ion concentrations, when compared with to one or more suitable controls is taken to be indicative of productive TCR:MHC antigen interactions and more specifically, presence of the immune complex. One or a combination of different strategies can be used to detect changes in intracellular ion concentrations in a particular assay format. In a more particular example, intracellular calcium ion mobilization is detected in using a fluorescein-based reagent, such as Fluo-3 AM, which increases its fluorescence intensity approximately 100-fold upon binding Ca2+. High throughput screening methods to detect intracellular calcium mobilization using the Fluo-3 AM reagent and a fluorometric imaging plate reader (See, for example, Jurewicz, A. et al. (1999) Genet. Eng. News 19, 44) can be used to study cellular responses in T cells mediated by the productive TCR:MHC antigen complex formation.

As will be readily apparent to those of skill in this particular field, choice of a particular detection format will be guided by recognized parameters including the TCR and MHC molecules of interest, the affinity and/or affinity of those molecules for each other, and the level of assay specificity required for optimal detection of the test compounds.

In a particular embodiment, practice of the present invention for the specific detection of particular immune complexes may be aided by the inclusion of suitable internal controls. Such internal controls may include control complexes formed between the TCR and non-cognate MHC antigen molecule or between the MHC antigen and a non-cognate TCR. Generally stated, suitable non-cognate molecules can be naturally-occurring or recombinant and can usefully encompass a range of fully soluble or cell-anchored (membrane-bound) molecules. The amount of the control complex formed represents the level of nonspecific association between the TCR (or MHC antigen) and its non-cognate pair and can be used as the background control value when evaluating specific interactions between the TCR and cognate MHC antigen molecule.

In embodiments in which what has been referred to as “high throughput” or “ultra throughput” screening methods are desired, particular detection formats may be very useful. Examples of such formats include, but are not limited to, cell free systems such as those embodiments of the invention using fully soluble TCR and MHC molecules. More specific examples of such formats include, but are not limited to, cell free assays based on optical, fluorescence or luminescence intensity, time resolved fluorescence, proximity-based energy transfer (including fluorescence resonance energy transfer assays, scintillation proximity assays and luminescence proximity assays), fluorescence polarization spectroscopy, and fluorescence correlation spectroscopy. See e.g., Silverman, L. et al. (1998) Curr. Opin. in Chem. Biol. 2: 397-403.

A variety of cell based detection formats have also been disclosed that can be used in accord with this invention. Such formats more specifically encompass cell-based assays, including, but not limited to, optical, fluorescence or luminescence intensity-based assays, fluorescent resonance energy transfer, time resolved fluorescence, luminescent and scintillation proximity assays, and what is known as two-hybrid and three-hybrid systems. See e.g., Fernandes, P. B (1998) Curr. Opin. in Chem. Biol. 2: 597-603; Gonzalez, J. E. and Negulescu, P. A. (1998) Curr. Opin. in Chem. Biol. 9: 624-631; Jenh, C-H. (1998) Analytical Biochem. 256: 47-55; Wu, P. et al. (1997) Analytical Biochem. 249: 29-36.

Additionally suitable detection methods particularly in embodiments using cell based formats include fluorescence activated cell sorting (FACS) techniques. See e.g., Altman, J. D. et al. (1996) Science 274: 94-96.

Compounds detected and identified by the method may be useful, for example, in decreasing or increasing formation of the immune complex that includes the TCR and MHC antigen molecules; decreasing or increasing the stability of the immune complex, or decreasing or increasing the avidity of the TCR or MHC antigen molecule for the other. Other uses of the detected compounds include use in the modulation of in vivo TCRs and MHC antigen molecules. More specific in vivo methods including methods for modulating an immune response in vivo are discussed below.

Once a particular detection format (or group of formats) has been selected, an important consideration in performing the methods of this invention is the appropriate preparation of at least one reaction mixture including at least one TCR molecule and at least one MHC antigen molecule of interest. In one embodiment, the MHC antigen molecule consists of an MHC component with a covalently linked (i.e. fused) presenting peptide. However in other embodiments it may be more useful to generate a peptide-MHC complex by loading the peptide onto the MHC molecule by recognized methods. In other embodiments, the MHC component can contain a covalently linked superantigen. Alternatively, the MHC component can be complexed to the superantigen chain by recognized methods. At least one test compound is added to the mixture and the mixture is treated under conditions sufficient to allow the TCR and MHC antigen molecules to interact. Preferably, and in the absence of a test compound that antagonizes TCR:MHC antigen interactions, the TCR and MHC antigen molecules form a plurality of specific non-covalent bonds that facilitate formation of the immune complex. If desired, that immune complex can be removed from the reaction mixture and analyzed, e.g., by methods already discussed. If desired, the immune complex can be detected using sedimentation centrifugation, electrophoresis, filtration, chromatography or a related molecular sizing technique.

The particular TCR and MHC antigen molecules employed in methods of this invention will vary depending on recognized parameters, e.g., the goals of the screening assay, the sensitivity desired, the size of the pool of test compounds, and whether a fully soluble or cell-based screening format has been selected.

For example, in many embodiments, it will be useful to express the TCR molecule using a cell based approach including use of specific cells provided in the Examples, which follow. In this example of the invention, the MHC antigen molecule can be fully soluble although the molecule can be expressed by a suitable APC or other cell as needed. In embodiments in which the TCR molecule and/or the MHC antigen molecules include full-length or nearly full-length chains, detection of the immune complex is facilitated by detecting the TCR molecule, the MHC antigen molecule; or both molecules, using, e.g., the direct or indirect detection formats already mentioned.

More particular methods of this invention can be conducted using one or a combination of specific reaction formats. In one embodiment of the method, a suitable MHC antigen molecule is immobilized onto a solid support or phase. The immobilized MHC antigen molecule is subsequently contacted by an appropriate TCR molecule, usually its cognate, which can be detectably labeled if desired. At least one test compound is then brought into contact with the TCR and MHC antigen molecules, although in some instances it may be useful to add the test compound prior to adding the TCR molecule including prior to immobilizing the MHC antigen molecule to the solid phase. Alternatively, the test compound can be added after formation of the immune complex. As a control, the TCR molecule, is added to another reaction chamber in which the MHC antigen molecule is bound to the same or similar solid support. However instead of adding the test compound, an aliquot of a suitable blanking solution such as water, buffer or the like is added usually in a volume similar to that used in the experimental reaction.

Next, the immune complex is detected as in the experimental reaction. In these embodiments of the method, detection of the immune complex can be achieved using direct or indirect detection formats as needed. For example, the TCR molecule, MHC antigen molecule, test compound; or all three molecules may be directly or indirectly labeled to facilitate good detection of the immune complex. Test compounds are then selected by criteria discussed above including capacity to increase or decrease formation and/or stability of the immune complex.

By the phrase “solid support” or “solid phase” is meant nearly any support capable of binding a TCR or MHC antigen molecule. Well-known supports or carriers include glass, polystyrene, polypropylene, polyethylene, dextran, nylon, amylases, natural and modified celluloses, polyacrylamides, gabbros, magnetite, dendrimers and silicon wafers. The nature of the carrier can be either soluble to some extent or insoluble for the purposes of the present invention. The support material may have virtually any possible structural configuration so long as the coupled molecule is capable of binding to an antigen or antibody. Thus, the support configuration may be spherical, as in a bead, or cylindrical, as in the inside surface of a test tube, or the external surface of a rod. Alternatively, the surface may be flat such as a sheet, test strip, etc. Preferred supports include polystyrene beads. Bead-based reagents are commercially available in a wide variety of formats that allow for high capacity binding/capture of recombinant proteins, efficient detection and the use of different separation and homogeneous assay methods. For many high throughput screening methods, bead-based reagents are advantageous. Those skilled in the field of the invention will know many other suitable carriers for binding TCR and MHC antigen molecules, or will be able to ascertain the same by use of routine experimentation.

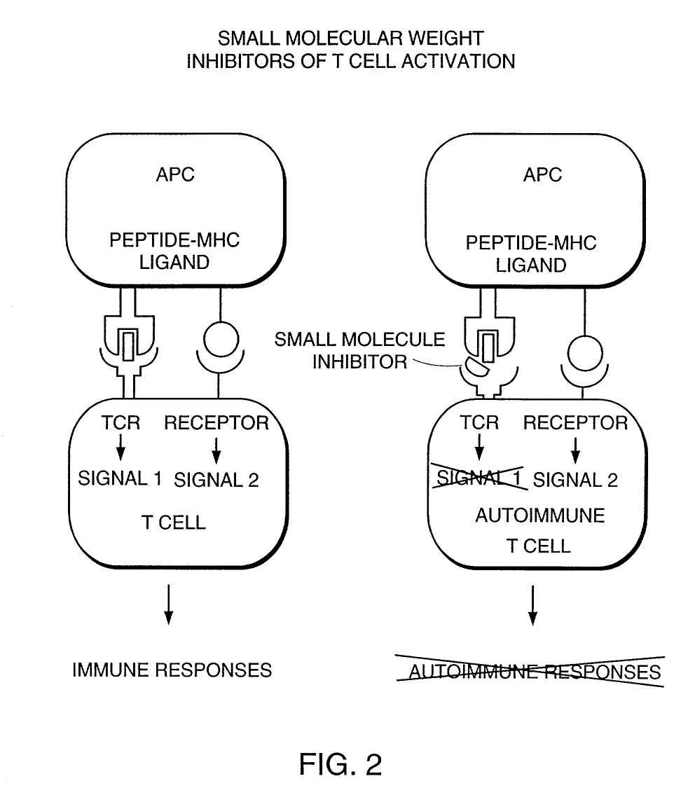

An illustrative example of the invention is represented schematically in FIG. 2. In FIG. 2, a small molecule inhibitor substantially blocks interaction between an MHC antigen consisting of a peptide-MHC ligand of an APC and a TCR expressed by a suitable cell. In this embodiment, the TCR:MHC antigen interaction is assisted by another receptor such as a CD molecule. Presence of the small molecule inhibitor blocks a subject immune response. However in the absence of the small molecule inhibitor that immune response is manifested.

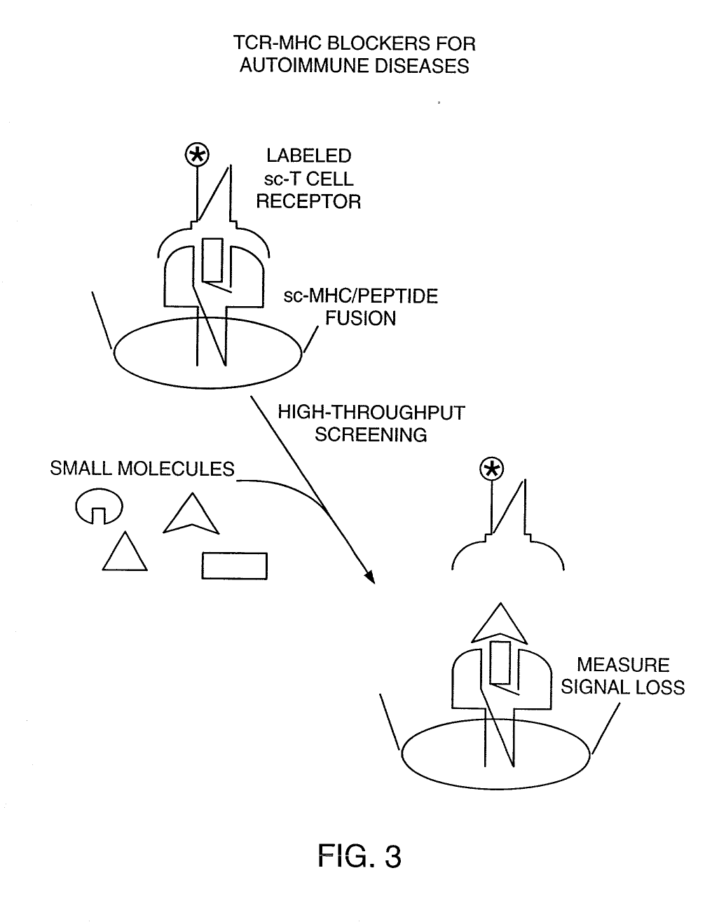

Another embodiment of the invention is shown in FIG. 3. In this example, a subject TCR molecule is detectably labeled and the test molecules are added to the reaction in a “high throughput” or “ultra throughput” screening format. It is emphasized however that such high volume screening is not necessary to practice this invention but may help analyze large pools of test compounds. In this example of the invention, a plurality of reaction chambers will often be used in which each reaction chamber includes the detectably labeled TCR molecule, the immobilized MHC antigen molecule and at least one and preferably about one test molecule per reaction chamber. A test compound detected in accord with this embodiment will facilitate a significant loss of signal indicative of a corresponding reduction in the presence of the immune complex in the presence of the test compound. In some embodiments, pools of compounds can be used where the concentration of each compound in the pool exceeds the limits of detection of the assay.

Alternatively, the particular screening method shown in FIG. 3 can be adapted so that the MHC antigen molecule is detectably labeled and the TCR molecule is immobilized to the solid support or phase. In another embodiment, the test compound can be detectably labeled instead of or in addition to the TCR molecule and/or the MHC antigen molecule. Choice of a particular screening paradigm will be guided by parameters already discussed including the goals of the screening assay and the level of sensitivity needed.

For example, the screen illustrated in FIG. 3 could be readily adapted to detect antagonists of a specific autoimmune disorder. In this embodiment, the TCR and MHC antigen molecules employed in the screen will be pre-selected to consist of those recognized to participate in or are suspected of participating in the immune disorder. Examples of such TCR t and MHC antigen molecules are provided herein including the Examples and discussion, which follows.

It is emphasized that the precise order of manipulating any of the TCR molecules, MHC antigen molecules, or test compounds in accord with this invention is usually not important so long as desired screening results can be readily met. That is, when it is desirable to test compounds for capacity to modulate formation of the immune complex, it will often be very useful to add those compounds prior to or during addition of the TCR and MHC antigen molecules. However in embodiments in which the screening method is designed to detect test compounds that modulate previously formed immune complexes, the test compounds can be added prior to, during, or after the TCR and MHC antigen molecules are combined as required. Thus the order of addition of components in a particular assay setting will be guided by recognized parameters including the specific objectives of the screening format implemented.

In many instances, what is referred to in the field as “titre” or “microtiter” plate may conveniently be utilized as the solid phase. A 96 well microtitre plate will often be preferred however plates with more wells may be indicated for more comprehensive screening paradigms. The anchored component may be immobilized by non-covalent or covalent attachment in accordance with accepted practice. For example, non-covalent attachment may be accomplished by simply coating the solid surface with a solution of the TCR or MHC antigen protein for an amount of time necessary to bind same to the solid phase. Alternatively, an immobilized antibody, preferably a monoclonal antibody, specific for the protein to be immobilized may be used to anchor the protein to the solid surface. The surfaces may be prepared in advance and stored if needed.

More specifically, practice of the invention can involve adding the nonimmobilized component to the coated surface containing the anchored component (which may be the TCR molecule or the MHC antigen molecule). After the reaction is complete, unreacted components are removed (e.g., by washing) under conditions such that any complexes formed will remain immobilized on the solid surface. The detection of complexes anchored on the solid surface can be accomplished in a number of ways that involve direct or indirect detection formats. Where the previously nonimmobilized component is pre-labeled, the detection of label immobilized on the surface indicates that immune complexes between the TCR and MHC antigen molecules was productive. Where the previously nonimmobilized component is not pre-labeled, an indirect label can be used to detect complexes anchored on the surface; e.g., using a labeled antibody specific for the previously nonimmobilized component (the antibody, in turn, may be directly labeled or indirectly labeled with a labeled anti-Ig antibody).

In another embodiment of the invention, a suitable MHC antigen molecule is immobilized onto a suitable solid phase such as a 96 well microtitre plate. In this example, the MHC antigen molecule consists of an MHC component that carries a covalently fused presenting peptide. Alternatively, the MHC antigen molecule can consist of a complex in which case the presenting peptide is preferably loaded onto the MHC component before conducting additional manipulations. In other embodiments, the MHC antigen can consist of an MHC component complexed with a covalently linked or unlinked superantigen. After about 12 to about 48 hours of incubation to help maximize the immobilization, the MHC antigen molecule is removed from the plate and the wells filled with a suitable cell culture medium such as DMEM, IMDM and the like. Next, the test compound is added to each well before or during addition of cells expressing the cognate TCR molecule. The cells are then incubated for a time sufficient to respond to the bound MHC antigen molecule, generally between from about a few hours up to about 3 days or more. A preferred cell response for detecting presence of the immune complex is cytokine production, and preferably IL-2 production.

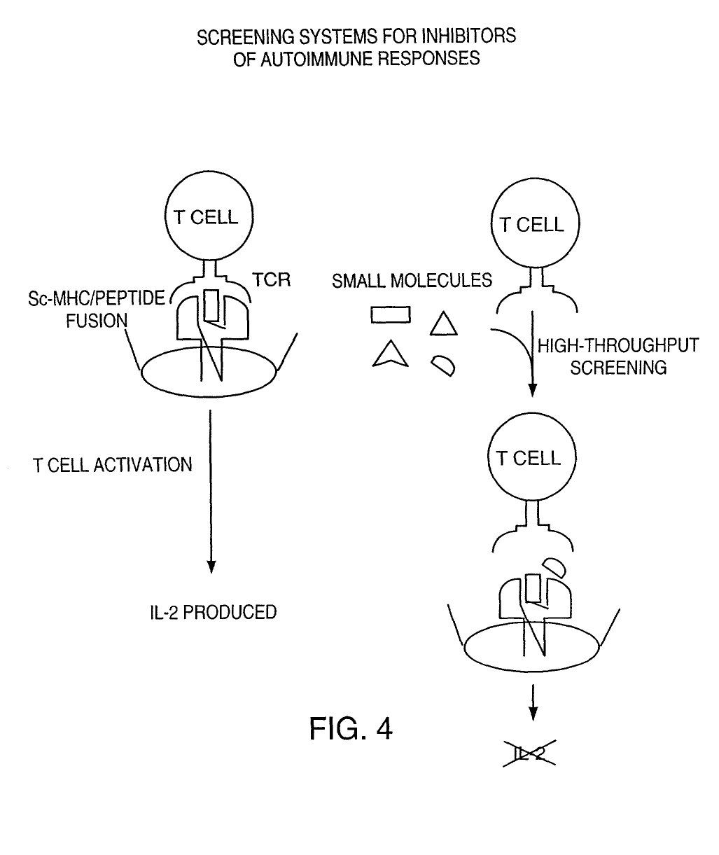

For example, FIG. 4 shows a desired MHC antigen molecule immobilized on a solid support such as the multi-well plates mentioned previously. In this embodiment, the MHC antigen molecules consist of an MHC component that is covalently linked presenting peptide. However as has been mentioned, empty MHC molecules can be used in this method in embodiments in which loading with the presenting peptide is preferred. In other embodiments, the MHC antigen can consist of an MHC component complexed with a covalently linked or unlinked superantigen. In this example of the invention, suitable cells expressing a desired TCR molecule such as naturally-occurring T cells or recombinant T cell hybridomas, are contacted to the immobilized MHC antigen molecules. At least one or more test compounds can be screened according to a desired format, preferably one of the “high throughput” or “ultra throughput” already discussed. The test compounds can be added at nearly any point of the assay including before, during or after addition of the cells to the wells or immobilization of the MHC antigen molecule to those wells. In this illustration of the invention, the monitored cell response is production of IL-2. As can also be seen in FIG. 4, in the absence of a suitable test compound, specific binding between the TCR and the MHC antigen molecules is productive and leads to formation of an immune complex. That immune complex triggers T cell activation and production of the IL-2 cytokine. However, binding of a suitable test compound such as an antagonist of formation of the immune complex reduces the cell response and resulting IL-2 production from the T cells. The test compound is thus detected and selected from the other compounds by observing a reduction in the cytokine production.

In a more particular embodiment, the cytokine produced by the cells and secreted into the culture media is detected by transferring the culture media to wells that had been coated with a antibody specific to the cytokine. After an appropriate incubation period to allow binding of the cytokine to the immobilized antibody (i.e. 2 hours at 25° C.), the culture media in each well is removed (by washing in PBS) and a detectably labeled antibody is added to the wells which antibody preferably binds the cytokine molecule. After incubating the labeled antibody, for example, for about a few hours up to a day or so, the wells are treated by one or a combination of different reagents suited to visualize binding of the labeled antibody to the cytokine molecule. A number of acceptable visualization formats have already mentioned including those involving generation of a chromogen. As mentioned, one cytokine is interleukin-2 (IL-2) although the present screening methods are compatible with the detection of other detectable molecules indicative of the cell response including other cytokines.

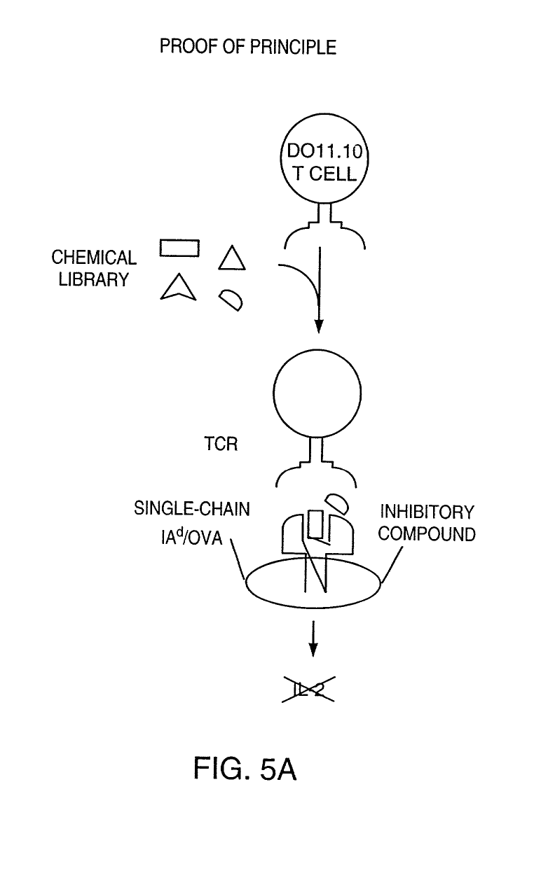

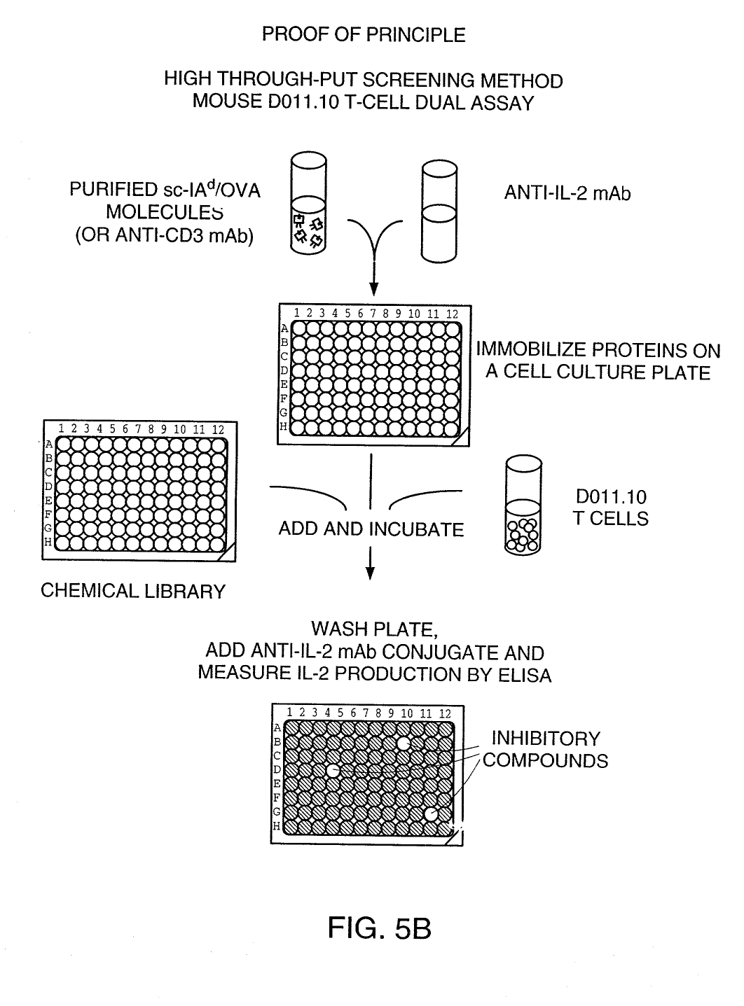

Another embodiment of the invention is intended to be one example of “proof of principle”. It is shown schematically in FIG. 5A. In this example, T cell hybridomas are used that expresses a DO11.10 T cell receptor. The MHC antigen molecule is bound, e.g., to the wells of the multi-well plate and in this example is a (sc-) IA^{d}/OVA protein which specifically interacts with the DO11.10 TCR. Suitable test compounds, e.g., provided in a “high throughput” or “ultra throughput” format, are added before, during or after addition of the recombinant T 1151 cells. In this illustration, the monitored cell response is production of IL-2.

As can be also be seen in FIG. 5A, in the presence of an appropriate test compound, specific binding between the DO11.10 TCR and the sc-IA^{d}/OV AMHC antigen molecule is blocked, thereby preventing formation of the immune complex. Absence of sufficient immune complex reduces or prevents T cell activation and production of the IL-2 cytokine. A reduction in the amount of the IL-2 cytokine can be readily detected using a variety of suitable direct or indirect formats.

Another embodiment of the screening method shown in FIG. 5A is shown schematically in FIG. 5B. This example of the invention is sometimes referred to as a “dual” assay format to designate capacity of the method to perform T cell stimulation and to measure T cell responses in one or a few wells. For example, in a more particular embodiment, a suitable MHC antigen molecule such the purified sc-IA^{d}/OVA is immobilized onto a multi-well culture plate along with a suitable amount of anti-IL-2 monoclonal antibody (i.e. capture antibody). Each well of the plate includes at least one test compound and typically about one of such compounds. Subsequently, T cells expressing the DO11.10 TCR are added to each well and after providing a sufficient incubation time, are washed away. Next, IL-2 production is measured in each of the wells by adding a suitable amount of detectably labeled antibody capable of specifically binding the IL-2 immobilized by the capture antibody. Typically, an ELISA detection format will be preferred although other immunological detection formats may be used as needed for specific test formats. As can be seen in FIG. 5B, inhibitory compounds that act as antagonists are readily identified by measurement of optical absorbance.

A more specific example of the foregoing screens is discussed below in Example 10. In this illustration of the invention, about 2000 small molecules were screened from a commercially available chemical library. Significantly, Example 10 shows that thirty compounds were detected that showed much lower IL-2 absorbance readings, i.e., the compounds reduced formation and/or stability of the immune complex formed between the DO1.10 TCR and the sc-IA^{d}/OVA protein.

In another embodiment, a desired MHC antigen molecule is associated with the cell membrane. In this embodiment, the MHC antigen molecule can consist of an MHC component that is covalently linked to presenting peptide. However, MHC molecules can be used in this method in embodiments in which loading with the presenting peptide is preferred. In other embodiments, the MHC antigen can consist of an MHC component complexed with a covalently linked or unlinked superantigen. For example, MHC antigens can consist of MHC/peptide complexes present on APCs pulsed with particular peptide. Methods for preparing suitable peptides and pulsing same are known in this field. See e.g., Sette, A. et al. (1987) Nature 328:395-399 and Martin, R. et al. (1990) J. Immunol. 145:540-548. In this example of the invention, suitable cells expressing a desired TCR molecule such as naturally-occurring T cells or recombinant T cell hybridomas, are contacted to the MHC antigen molecules. At least one test compound can be screened according to a desired format. The test compounds can be added at nearly any point of the assay including before, during or after addition of the cells to the wells. In this illustration of the invention, the monitored cell response is production of IL-2. In the absence of a suitable test compound, specific binding between the TCR and the MHC antigen molecules is productive and leads to formation of an immune complex. That immune complex triggers T cell activation and production of the IL-2 cytokine. However, binding of a suitable test compound such as an antagonist of formation of the immune complex reduces the cell response and resulting IL-2 production from the T cells. The test compound is thus detected and selected from the other compounds by observing a reduction in the cytokine production.

Example 10 below provides a more specific illustration of this embodiment of the invention. In this example, A20 APCs, the OVA peptide and DO11.10 T cells are mixed to allow complex formation between the OVA peptide and the IA^{d} molecules on the A20 cells and stimulation of the DO11.10 T cells by the IA^{d}/OVA complex. In this example, stimulation of the DO11.10 T cells is measured by IL-2 cytokine production as described below. Test compounds were added to the OVA-pulsed A20:DO11.10 T cell mixture to assess the ability of the test compounds to affect formation of the immune complex and cytokine production. Significantly, Example 10 shows that four compounds were detected that showed much lower IL-2 absorbance readings, i.e., the compounds reduced formation and/or stability of the immune complex formed between the DO11.10 TCR and the IA^{d}/OVA complex.

As discussed, it is an object of this invention to provide highly efficient screening methods that can facilitate the detection of many test compounds. Preferably, detected test compounds specifically modulate the immune complex formed between cognate TCR and MHC antigen pairs.

In accord with this objective, the invention provides strategies for selecting against test compounds that can modulate cells nonspecifically, e.g., by impacting IL-2 production from cells. In this embodiment, any of the foregoing methods can be repeated and the effects of the detected compounds on non-specific T cell stimulation assayed.

For example, one way to assay for non-specific T cell stimulation is by using an antibody in the method that is capable of binding a cluster of differentiation (CD) proteins such as CD3. As discussed, the CD3 protein has been reported to be closely associated with many TCRs. In this example of the invention, the antibody can be immobilized onto the wells prior to, during, or after addition of the TCR or MHC antigen molecules. Presence of the immobilized antibody will generally activate T cells and stimulate them to produce a cytokine such as IL-2. Thus a test compound that interferes with the antibody mediated activation of the T cells is designated as one that does not specifically modulate the immune complex formed between the TCR and MHC antigen molecules. Although such test compounds may be of interest in some instances, more preferred test compounds of this invention will specifically modulate TCR:MHC antigen interactions as detected by presence or absence of the immune complex.

Turning to FIG. 5B again, one embodiment of this particular screen is shown in which an anti-CD3 antibody is used as a control. As discussed, sometimes this control will be referred as a “control immune complex”, “internal control” or similar phrase.

It is an important objective of this invention to provide methods that, if desired, can be adapted to detect test compounds that specifically modulate MHC antigen dependent stimulation of the T cells while having no or little impact on the ability of these cells to respond to stimulation through a TCR/CD complex such as TCR/CD3. A more specific illustration of the control immune complex is discussed below in Example 10. There, specific test compounds of interest modulate MHC antigen mediated stimulation of the TCR cells and have insignificant or negligible inhibition of IL-2 production mediated by the anti-CD3 antibody.

If desired, the present methods can be used with one or a combination of control immune complexes such as the TCR/CD3 complex already mentioned. As discussed, analysis of such control immune complexes in the presence and absence of test compounds can help refine a particular screen (assuming refinement is needed) and facilitate the detection of compounds that specifically modulate the immune complexes. For example, another control immune complex well suited for use with the present invention comprises an anti-TCR antibody, usually an anti-TCR monoclonal antibody. In this embodiment, the anti-TCR antibody preferably specifically binds to a TCR epitope outside the receptor binding site, i.e., it will not prohibit binding between the TCR and its cognate MHC antigen molecule as determined by conventional protein:protein binding experiments. Examples of such monoclonal antibodies are known in the field. See e.g., Haskins, K. et al. (1983) J. Exp. Med. 157: 1149-1169, Kubo, R. T. et al. (1989) J Immunol. 142:2736-2742, and Brenner, M. B. et al. (1984) J Exp. Med. 160:541-551. Preferred use of the anti-TCR antibody as a control immune complex is discussed below in Example 11.

In some instances such as when a desired test compound weakly modulates particular TCR and MHC antigen interactions in a given assay setting, it may be very useful to boost the sensitivity of that assay. Such enhancement can be achieved by one or a combination of different strategies including enhancing direct or indirect detection of the immune response using conventional methods. Alternatively, such sensitivity enhancement can be achieved by manipulating the TCR and/or MHC antigen molecules such as by providing those molecules as multi-valent complexes. Example 15 provides a particular illustration of this technique.

In one embodiment, assay sensitivity can be enhanced by increasing the ability of cells to respond to stimulation from the immune complex. In particular, there is recognition in the field that interactions between costimulatory receptors, such as certain CD molecules on the T cell surface, and their cognate ligands can stimulate the initiation and amplification of intracellular signals mediated by the TCR:MHC antigen interactions. Thus it is a further object of this invention to provide naturally-occurring or recombinant cells expressing desired TCR or MHC antigen molecules which cells further express at least one suitable costimulatory receptor, such as CD28 or LFA. In this embodiment of the invention, co-expression of the costimulatory receptor with the TCR and/or MHC antigen molecules can under the preferred assay conditions enhance the cellular responses to the immune complex, thereby boosting assay sensitivity and reliability. Preferred assay conditions include, but are not limited to, the addition of the cognate ligands or antibodies (or fragments thereof) capable of binding the costimulatory receptor, wherein the cognate ligands or antibodies are added as soluble, immobilized or cell-associated molecules.

Another way to boost assay sensitivity is to provide for better presence of the immune complexes. In particular, there is recognition in the field that certain CD proteins can facilitate interaction with MHC antigen molecules. Thus it is a further object of this invention to provide naturally-occurring or recombinant cells expressing desired TCR or MHC antigen molecules which cells further express at least one suitable CD protein, e.g., CD4 or CD8. In this embodiment of the invention, co-expression of the CD protein with the TCR and/or MHC antigen molecules can facilitate formation of the immune complex, thereby boosting assay sensitivity and reliability.

In a particular embodiment of the method, cells are engineered to express a desired TCR molecule, e.g., a sc-TCR fusion molecule, and a CD4 protein or fragment thereof on the cell surface. In another embodiment, a desired TCR molecule is provided as a sc-TCR fusion molecule which molecule is engineered to include, as part of the fusion protein, at least a portion of another CD molecule such as CD3ζ or functional fragment thereof. In still another embodiment, the sc-TCR-CD3ζ construct is co-expressed with the CD molecule, usually CD4 or CD8. In many instances, co-expression of the TCR molecule with the CD protein(s) will allow the cells to be more sensitive to simulation by the MHC antigen molecule. In this example, the MHC antigen molecule can be provided as a fully soluble protein or in a cell-based format as required.

Example 12 below provides a more specific illustration of this embodiment of the invention. Here, the DO11.10 TCR is present as a sc-CD3, fusion protein expressed by cells that also express a recombinant CD4 protein on the cell surface. A cognate MHC antigen molecule, scIA^{d}/OVA is immobilized onto suitable multi-well plates along with a suitable anti-IL-2 antibody, usually a monoclonal, generally for about 12-48 hours. That antibody can be added to the plates prior to, during, or along with the immobilization of the MHC antigen molecule. Subsequently, recombinant cells co-expressing the single chain TCR and CD4 proteins are added to the wells and cultured for several hours or up to a few days or more. IL-2 production by the cells is measured along lines described previously.

In an embodiment of the method, the MHC antigen constructs can be covalently linked to an immunoglobulin fragment such as IgM, IgG, IgA and the like; as well as a functional fragment thereof. If desired, control wells can be prepared in which cell stimulation is measured using different MHC antigen. If also desired, further controls may be conducted such as use of at least one of the internal controls described above including those described specifically in the Examples. In this embodiment of the invention, the capacity of a test compound to modulate stimulation of the recombinant cells will be measured as a corresponding increase or decrease in the amount of cytokine produced by the cells. Test compounds that inhibit T cell stimulation by the MHC antigen but do not inhibit stimulation by the anti-TCR antibody can be pursued as lead compounds.

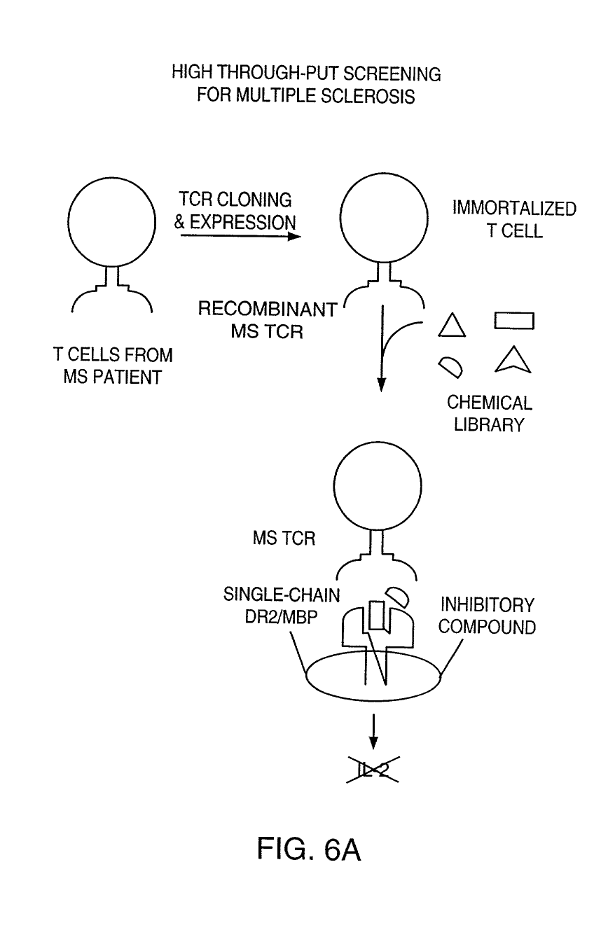

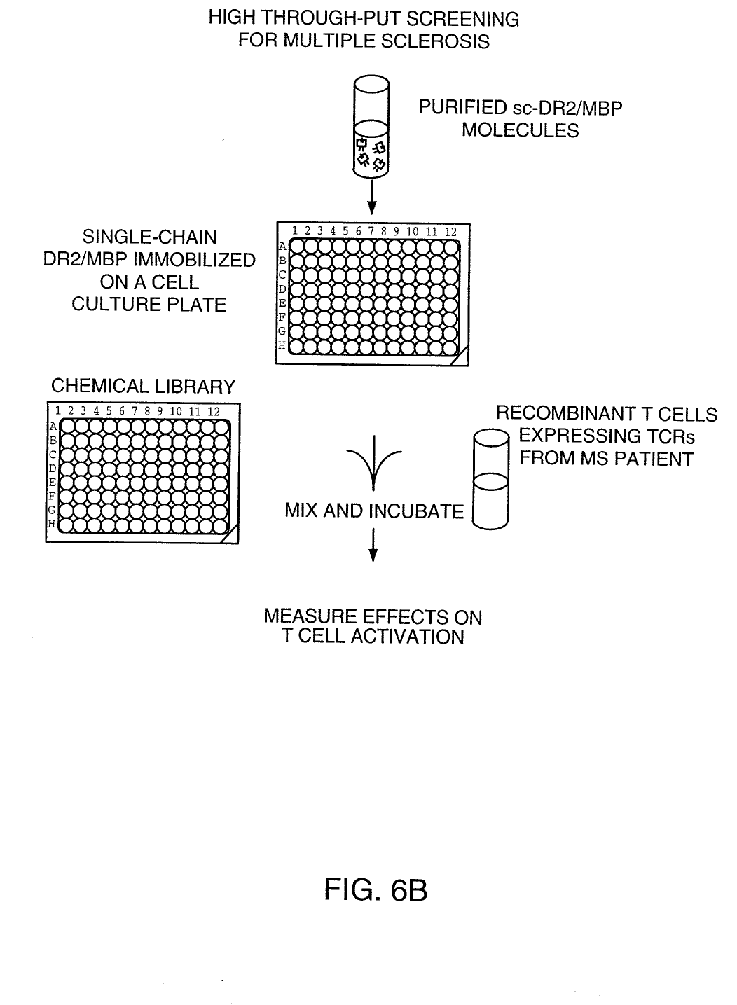

A specific example of this method is schematically shown in FIG. 6A. Here, the TCR and MHC antigen probes employed have been implicated in multiple sclerosis (MS); a common human autoimmune disorder resulting in severe axon demyelination. FIG. 6A shows an embodiment of the method in which test compounds are screened for those that modulate interactions with the MS TCR and MHC antigen probes. The T cell is preferably derived from T cells obtained from a human MS patient. Alternative, as shown, cells (i.e., recombinant MS TCR cells) are made by TCR cloning and expression steps such as those provided herein. The recombinant MS TCR cells are then added to the multi-well plates (having bound MHC antigen molecules) along lines discussed previously. In this illustration, at least one test compound is added to the wells, usually about one test compound, either before, during or after addition of the recombinant MS TCR cells. Preferably, the method is conducted in a high throughput screening format. In this example of the invention, the test compound is detected by a reduction or elimination of the amount of IL-2 produced by the MS TCR cells compared with at least one suitable control, e.g., the amount of IL-2 produced from the cells in the absence of the test compound.

As shown in FIG. 6B, purified sc-DR2/MBP (a single chain class II MHC with fused MBP presenting peptide) is immobilized onto a multi-well cell culture plate. Suitably, the plate includes a chemical library in which each well preferably includes at least one and usually about one test compound. Next, T cells expressing relevant TCRs are obtained by conventional means from a patient who is suffering from or is suspected of suffering from or susceptible to MS. In most cases however, the patient will be suffering from MS as indicated by recognized parameters. As an alternative, recombinant cells expressing relevant TCRs can be generated. A suitable amount of the T cells are then added to each well as the screen is conducted along lines mentioned above. In this embodiment, the cell response is measured in the presence and absence of the test compounds from the chemical library which response can be cytokine production as mentioned previously.

Of course, the foregoing specific methods for detecting test compounds that module MS immune complexes can be readily adapted to screen other TCR and MHC antigen molecules of interest. A more specific example of the foregoing screening methods is provided by Example 13 below. In this example of the invention, suitable T cell V-α and V-β chains are obtained from a specific human MS T cell line restricted for myelin basic protein (MBP). Nucleic acid encoding the chains can be fused, if desired, to a human CD3ζ sequence or functional fragment of that sequence. The resulting construct is then expressed in suitable cells and particularly immortalized T cells. Such transfected cells are then manipulated along lines already discussed. That is, the immortalized T cells are added to multi-well plates in which each well includes immobilized or cell expressed cognate MHC antigen, e.g., MBP peptide-loaded DR2⁺APCs. Alternatively, MHC antigen molecules can be generated in which the presenting peptide is covalently linked to the MHC as provided herein and in the U.S. Pat. No. 5,869,270. Thus, this is an embodiment of the invention in which the detection format is entirely cell based.

More particularly, Example 13 provides a detection format in which the MS restricted TCR is cell based and the cognate MHC antigen molecule, (sc-DR2+/MBP) is immobilized onto the wells. In this example of the invention, the MHC antigen protein can be fused to an acceptable immunoglobulin molecule such as IgM, IgG, IgA and the like; or a fragment thereof. After immobilizing the scDR2/MBP molecules to the wells for a few hours up to a day or so, the recombinant T cells are added to the wells in medium and incubated therein for a few hours up to a few days. Each of the wells also includes at least one and often one test compound provided in a suitable solvent. The test compound may be added at any point in the assay including prior to, during or after addition of the T cells or immobilization of the MHC antigen molecule. However, addition of the test compound along with the T cells is generally preferred for this illustration of the invention. Capacity of the compounds to modulate interaction between the MHC antigen and the TCR molecule can be monitored directly or indirectly as described earlier. Also in this example, the production of IL-2 is measured along lines discussed. If desired, this particular method can include one or more controls, including incubation of the recombinant cells in the absence of the test compounds. Additional controls that can also be employed include those control immune complexes described earlier. Particular test compounds detected by the method will significantly modulate interactions between the TCR and MHC antigen molecules but will not modulate stimulation by the anti-TCR or anti-CD3 antibodies described above. Preferred are test compounds that show substantial antagonistic activity in this assay.

In another embodiment, the invention provides cell based screening methods in which cells are designed to express at least two different antigen specific TCRs on the surface of the cells. This particular example of the invention provides advantages. For example, it often allows the method to be more sensitive to test compounds that show distinct effects toward different TCRs. As in the illustration, the test compounds may exhibit capacity to modulate one of the TCR molecules while modulating the other much less so or not at all.

By way of illustration of this embodiment, suitable cells such as immortalized T cells are engineered to express a desired (first) TCR molecule along with what is sometimes referred to herein as a “control” or “second” TCR molecule. Such cells could then be used along lines discussed previously, e.g., with APCs expressing an MHC antigen molecule of interest or with immobilized MHC antigen molecules. Capacity of the compounds to modulate interaction between the MHC antigen and the first TCR molecule can be monitored directly or indirectly as already described. Such interactions can then be compared with capacity of the compounds to modulate specific interaction between the second TCR molecule and its cognate MHC antigen. In many instances, it will be desirable to measure cytokine production, including secretion of IL-2 by cells according to methods already outlined. Particular compounds of interest will modulate interaction between the first TCR and its cognate MHC antigen while providing much less or no modulation between the second TCR molecule and its cognate MHC antigen.

Accordingly, it is an object of this invention to provide methods that include, as needed, at least one suitable internal control. Thus any one of the foregoing methods can include at least one the following steps:

1) contacting a subject TCR molecule, e.g., the first TCR molecule with at least one antibody, preferably a monoclonal antibody, capable of specifically binding the TCR preferably at one or more epitopes outside the sites bound by the MHC antigen molecule, the contacting being in the presence or absence of the test compound and under conditions sufficient to bind the TCR and the antibody as a control immune complex,

2) detecting presence of the control immune complex in the presence and absence of the test compound; and

3) selecting a test compound that does not detectably alter (increase or decrease) specific binding between the TCR and antibody.

In another embodiment, the methods can include, as needed, at least one the following steps:

1) contacting a second TCR molecule with its cognate MHC antigen molecule, the contacting being in the presence or absence of the test compound and under conditions sufficient to bind the second TCR and the MHC antigen as a control immune complex,

2) detecting presence of the control immune complex in the presence and absence of the test compound; and

3) selecting a test compound that does not detectably alter (increase or decrease) specific binding between the second TCR and MHC antigen molecule. Preferably, the second TCR has a different MHC antigen binding specificity than the subject (first) TCR molecule indicated above.

As discussed, the choice of whether or not to include one or more internal controls will be guided by recognized parameters such as the test compounds to be screened and the amount of sensitivity and selectivity required.

Example 14 below provides another specific illustration of the invention particularly with respect to including an internal control. In this example, the first TCR molecule is a single chain DO11.10 TCR molecule and the second TCR molecule is an endogenous TCR. To evaluate responsiveness of the cells to stimulation through the first and second TCR molecules, the cells are incubated in appropriate multi-well plates that include a suitable control MHC antigen molecule. For example, suitable control MHC antigens can consist of MHC/peptide complexes present on the appropriate APCs pulsed with particular peptide. Methods for preparing suitable peptides and pulsing same are known in this field. See e.g., Sette, A. et al. (1987) Nature 328:395-399, Martin, R. et al. (1990) J. Immunol. 145:540-548. Preferably, that peptide when presented in the context of the APCs predominantly stimulates the second TCR but not the first TCR. The cognate MHC antigen molecule for the first TCR is usually immobilized to the wells as outlined previously. The MHC antigen molecule can consist of an MHC component that carries a fused presenting peptide or is loaded with a peptide as desired. That peptide is preferably specific for the first TCR molecule and not the second TCR molecule. In other embodiments, the MHC antigen can consist of an MHC component complexed with a covalently linked or unlinked superantigen. That superantigen is preferably specific for the first TCR molecule and not the second TCR molecule.

In this example of the invention, at least one test compound can be added to the wells along lines previously discussed including before, during or after addition of the APCs or immobilization of the MHC antigen molecules. As an illustration, the test compound can be added after immobilizing the MHC antigen molecules and before adding cells expressing the 1 TCR molecules. A preferred cell response is cytokine production and particularly IL-2 production although in some instances it may be useful to monitor other cells responses such as those mentioned herein. In this embodiment, particular test compounds of interest will modulate interactions between the first TCR and its MHC antigen while providing little or no modulation between the second TCR and its MHC antigen. More particular test compounds of interest will show good inhibition of interactions between the first TCR and its cognate MHC antigen but will not show similar inhibition with respect to the second TCR and its cognate MHC antigen.

In more specific embodiment, the present invention encompasses a screening method that utilizes cell lines reconstituted with at least two TCR molecules preferably restricted by different MHC antigens. This feature of the invention provides numerous advantages including minimizing any assay variability.

A number of the examples below provide another specific illustration of the invention particularly with respect to including additional internal controls. For example, internal controls may include control complexes formed between the TCR and non-cognate MHC antigen molecule or between the MHC antigen and a non-cognate TCR. As described, these control complexes represent the nonspecific association between the TCR (or MHC antigen) and its non-cognate pair and can be useful as a negative control to evaluate the level of specific interactions between the TCR and cognate MHC antigen molecule. For example, particular embodiments illustrated in Examples 3, 5, 11, 12, 14, 15 and 16 utilize suitable MHC antigen molecules for establishing the nonspecific association between a TCR and a non-cognate MHC antigen molecule. In each of these examples, the non-cognate MHC antigen is a peptide-MHC complex carrying a peptide that differs from the cognate peptide specifically recognized by the TCR. Other embodiments illustrated in Examples 11, 14, and 15 utilize suitable TCRs for establishing the nonspecific association between an MHC antigen molecule and a non-cognate TCR.

Practice of the present invention provides many advantages particularly in light of prior cell-based screening attempts (See i.e., WO 96/36881). For example, the methods of this invention are fully compatible with a wide range of TCR and MHC antigen molecules. This feature of the invention is important as it expands the repertoire of cell- and protein-based assay formats for detecting the test compounds. In contrast to prior screening attempts, the method of the invention also provides for a number of internal controls that select against test compounds that modulate cell responses nonspecifically. As described, one of the major limitations with cell-based screening assays in general has been the detection of “false positive” compounds that modulate the measured cellular response nonspecifically or indirectly. Preferred use of the present invention substantially reduces and in some cases eliminates false positive or misleading test results.

As discussed, one way to boost or enhance the sensitivity of the present methods is to provide subject TCR molecules and/or MHC antigen molecules as multi-valent complexes. In this embodiment, the invention can provide significant advantages. For example, it is recognized in the field that multi-valent MHC antigen molecules can stimulate T cell responses to a greater degree, in some cases to a much greater degree, than monomeric MHC antigen molecules. See Abastado, J. (1995) J. Exp. Med. 182:439-447 and Hamad, A. R. et al. (1998) J. Exp. Med. 188:1633-1640.

For example, practice of the methods with multi-valent TCR and/or MHC antigen molecules can desirably increase the avidity of weakly interacting TCR:MHC antigen complexes. One way to achieve this result is to covalently link or fuse at least one “adapter” or “tag” molecule to the TCR molecule, the MHC antigen molecule, or both molecules. These adapters or tags act to directly or indirectly promote the formation of multi-valent complexes. Illustrative adapters or tags include biotin, streptavidin and immunoglobulin molecules such as immunoglobulin variable and/or constant domains including IgG and IgM heavy chains, κ and λ chain s; or functional fragments thereof. Particular adapters of interest can be specifically bound by another molecule including protein A, protein G, biotin, streptavidin or an antibody such as a monoclonal antibody. The antibody may be detectably-labeled or not as required. Other adapters of interest are protein domains that are capable of associating to form multimeric complexes, such as leucine zipper domains. Multi-valent forms of the MHC antigen and TCR molecules may also be generated by chemically cross-linking the molecules, either directly or indirectly. Further descriptions of the multi-valent forms of the MHC antigen molecule of the invention can be found in the U.S. patent application Ser. No. 08/960,190.

Further descriptions of the multi-valent forms of the TCRs of the invention can be found in a U.S. patent application Ser. No. 09/422,375 entitled Polyspecific Binding Molecules and Uses Thereof, filed on Oct. 21, 1999; the disclosure of which has already been incorporated by reference.

See also the U.S. patent application Ser. No. 09/422,375 for disclosure relating to other MHC antigen molecules and TCR molecules for use with this invention including multi-valent forms of such molecules.

For example, in one embodiment, a subject MHC antigen molecule can be fused by standard methods to a suitable adapter or tag molecule, e.g., an immunoglobulin constant domain such as IgG or a functional fragment thereof. In a particular example, the MHC antigen molecule is preferably configured as single chain MHC construct that includes a fused presenting peptide and the adapter. Usually the adapter or tag will be fused to the C-terminus of the single chain construct although other arrangements such as fusion to the N-terminus may be indicated for some applications. Particular multi-valent complexes will generally have about 2 to about 10 units, preferably about 2 to 5 units, with about 4 units (i.e. a tetramer) being useful for many applications.

Examples 3 and 5 below describe more specific methods of the invention using multi-valent complexes to enhance the sensitivity of the assays detecting formation of immune complexes. In these examples, multi-valent MHC antigens were generated and immobilized on a solid support i.e. multi-well plates as described previously. In this embodiment, the MHC antigen molecule is an MHC component that is covalently linked to presenting peptide. However as has been mentioned, empty MHC molecules can be used in this method in embodiments in which loading with the presenting peptide is preferred. In other embodiments, the MHC antigen can consist of an MHC component complexed with a covalently linked or unlinked superantigen. Different methods can be used to generate multi-valent complexes as described. In Example 3, the MHC antigen molecule, scIA^{d}/OVA, was fused to the immunoglobulin constant domains of the IgG heavy chain and kappa chains. In Example 4, the scIA^{d}/OVA molecule was fused to the immunoglobulin constant domains of the IgM heavy chain. In both examples, multimerization of the scIA^{d}/OVA molecules was mediated by the immunoglobulin scaffold. In this embodiment of the invention, cells expressing the DO11.10 TCR are added to the wells and cultured for several hours or up to a few days or more. IL-2 production by the cells is measured along lines described previously. As described in Examples 3 and 5 below, a significant enhancement (2 to 10 fold) in assay sensitivity is provided by use of multi-valent complexes.

In another embodiment in which the multi-valent MHC antigen molecules are used, cells expressing a cognate TCR molecule are contacted by at least one fully soluble multi-valent MHC antigen molecule, typically in an overnight incubation. Test compounds can be added to the wells along lines previously discussed including before, during or after addition of the cells or the multi-valent MHC antigen molecule. Subsequently, the cells are washed and the cells are contacted by an detectably labeled antibody capable of specifically binding the multi-valent MHC antigen molecule (now bound to the cells). The cells are then analyzed for presence or absence of the detectable label. Presence of the detectable label is taken to be indicative of formation of TCR:MHC antigen complexes. Particular test compounds of interest are those that show capacity to modulate formation of the immune complexes when compared to one or more of those controls mentioned herein.

A more specific example of the method is the plate-based assay format described below in Example 15. In this illustration of the invention, the MHC antigen molecule is a sc-IA^{d}/GD-Ig fusion protein and the cells are T cell hybridomas that express the DO11.10 or GD12 TCR. The multi-valent MHC antigen molecules are prepared in the method by incubation with an anti-Ig monoclonal antibody detectably labeled with biotin. That biotin is itself tagged by further incubation with streptavidin linked to cytochrome. In this particular example of the invention, the control performed is the co-incubation of the multi-valent MHC antigen with cells expressing the DO11.10 T cells. As explained in the Example 15, flow cytometry (i.e. FACS) of the stained cells indicated that the scIA^{d}/GD-Ig molecules specifically stained the GD 12 T cell hybridoma but failed to stain cells expressing the DO11.10 cells.

The embodiment of the invention exemplified by Example 15 can be adapted to screen test compounds in a variety of screening formats such as those high or ultra throughput assays mentioned above and in the Examples below. Typically, test compounds will be identified and selected for capacity to specifically modulate interaction between the GD12 T cell hybridoma and its cognate multi-valent MHC antigen molecule. Particular test compounds of interest will manifest a readily detectable alteration (increase or decrease) in the cell signal. This specific assay can be further adapted to screen other TCR:MHC antigen pairs and, if desired, the DO11.10 cells can be employed as a useful control for those test compounds which modulate the TCR:MHC antigen interactions non-specifically.

In another embodiment, the invention provides methods that employ soluble molecules including fully soluble TCR and MHC antigen molecules. In one embodiment, the invention particularly provides protein-based screening methods for detecting test compounds that modulate TCR:MHC antigen interactions. This feature of the invention can provide important advantages including reducing or eliminating “false positive” signals that can in some instances emanate from non-specific effects on cell responses. Typically, approaches that include ELISA, fluorescence-based and homogeneous assay formats will be preferred although other direct or indirect detection formats may be more useful in particular screening paradigms.

In this particular example of an ELISA-based format of the invention, the screen includes three useful components: 1. the soluble TCR which recognizes a cognate MHC antigen molecule, 2. the soluble cognate MHC antigen molecule and 3. the inhibitory compound which blocks the specific interaction between the TCR and MHC antigen molecule. The assay, for this example, is formatted such that the TCR (1) is fixed in a well of a microtiter plate and the MHC antigen molecule (2) and the inhibitory compound (3) are added, allowed to bind the TCR (1) and the extent of binding is then disclosed by adding a labeled antibody or other agent which recognizes the MHC antigen and provides a readout through a chemical reaction (label=horseradish peroxidase [HRP] or alkaline phosphatase [AP]) or direct measurement (label radioactive isotope or fluorescent compound).

For example, in one approach, a recombinant cell is made that expresses a soluble subject TCR, e.g., in a recombinant single chain format. Suitable amounts of the sc-TCR are purified and then immobilized onto a solid support or phase such as a multi-well plate, usually for a few hours up to about 24 hours or more. Typically, each well of the plate will include the immobilized TCR molecule. Subsequently, a suitable amount of a detectably labeled MHC antigen molecule is added to each of the wells. In one embodiment, the detectable label is selected to provide for multimerization of the MHC antigen molecule although in other embodiments the label need not provide for multimerization. As needed, the MHC antigen molecule can consist of an MHC component carrying a fused presenting peptide. Alternatively, the MHC molecule can be empty in which case the molecule can be loaded with presenting peptide in accord with established procedures. In other embodiments, the MHC antigen can consist of an MHC component complexed with a covalently linked or unlinked superantigen. However in instances in which multimeric MHC antigen molecules are used it will often be possible to boost the avidity of weakly interacting complexes. Thus it will be readily apparent that this embodiment of the invention may be preferred where the subject immune complex is known or suspected of being weakly interacting.

In this example, test compounds can be added to the wells along lines previously discussed including before, during or after addition of the TCR or MHC antigen molecules. Particular test compounds of interest will desirably modulate specific binding between the TCR molecule and the multimerized or monomeric MHC antigen molecules.

A particular example of this embodiment is provided below in Example 16. In this embodiment, the screening method is designed to detect test compounds that are capable of modulating immune complexes formed between the 264 TCR and its cognate MHC antigen, the HLA-A2 class I molecule loaded with the peptide 264-272 fragment of the p53 human tumor suppressor protein.

Another particular example of this embodiment is provided below in Example 17. In this embodiment, the screening method is designed to detect test compounds that are capable of modulating autoimmune immune complexes formed between a single-chain TCR derived from an MS patient and its cognate MHC antigen, a single-chain HLA-DR2 molecule covalently linked to an MBP peptide.

The format of this assay can be varied widely. For instance, the MHC antigen can be fixed on the plate and probed with a soluble mono- or multi-valent TCR. Alternatively, the reaction may be formatted to run in solution by fixing one or the other of the reagents to beads or dendrimers to facilitate solution chemistry, capture or detection.

In a more specific illustration, the screen can be formatted as follows: the MHC antigen is attached to the plate, the plate is washed and blocked, soluble TCR is added and allowed to incubate at room temperature for 1 hour. The plate is washed, enzyme- or biotin-linked antibody is added and allowed to interact with the TCR complex for 30 min to 1 hr. The plate is washed, reprobed with enzyme-linked avidin (if necessary) and washed. A chromogenic enzyme substrate is added and the enzyme is allowed to convert the substrate to the chromophore product The reaction is stopped and results are collected on a plate reader measuring optical absorbance at the wavelength appropriate for the chromophore product. In the case where the MHC antigen consists of a peptide-MHC complex, the overall avidity of the TCR for the MHC antigen is the sum of the affinities of the TCR for the MHC without peptide or with an irrelevant peptide (Reich et al. (1997) Nature 387:617-620 measured the K_{d} of this interaction to be >2 mM for the 2B4 TCR and an empty IE^{k}), the affinity of the TCR for the MHC loaded with the appropriate peptide (Reich et al. ibid.—K_{d} of 2B4 for IE^{k}/MCC peptide=50-90 μM; Seibel et al. (1997) J. Exp. Med 185:1919-1927—K_{d} of DO11 for IA^{d}/OVA peptide=31 μM), and the additional stability provided by oligomer formation (Reich et al.). The valency of the TCR used will effect the overall assay conditions (O'Herrin S. M. et al. (1997) J. Exp. Med. 186:1333-1345 showed an approximately 50 fold higher affinity when using divalent vs. monovalent TCRs) and is one of the variables that requires refinement.

In another embodiment, the assay may be formatted as a homogeneous assay to avoid problems associated with sample transfer and wash steps. A homogeneous assay is preferred for high throughput and ultra high through put screening. Particularly preferred homogeneous assay are those that rely of detection of fluorescence or luminescence probes for the formation of the immune complex. More specific examples of such formats include, but are not limited to assays based on optical, fluorescence or luminescence intensity, time resolved fluorescence, proximity-based energy transfer (including fluorescence resonance energy transfer assays, scintillation proximity assays and luminescence proximity assays), fluorescence polarization spectroscopy, and fluorescence correlation spectroscopy.

Example 18 below provides a particular example of this embodiment. In this example, the screening method is designed to detect test compounds that are capable of modulating autoimmune immune complexes formed between a single-chain TCR derived from an MS patient and its cognate MHC antigen, a single-chain HLA-DR2 molecule covalently linked to an MBP peptide. The purified MS sc-TCR and sc-DR2/MBP proteins will be immobilized onto donor and acceptor beads, respectively. The acceptor beads are designed to emit a chemiluminescent signal when brought into close proximity of the donor beads by the interactions between the immobilized TCR and MHC antigen molecules. The TCR- and MHC antigen-coated beads will be added to microtiter wells and the luminescent signal resulting from productive formation of the immune complex will be measured in a time-resolved mode. Test compounds can be added to the wells along lines previously discussed including before, during or after addition of the TCR or MHC antigen molecules. Particular test compounds of interest will desirably modulate specific binding between the TCR molecule and the MHC antigen molecules.

Previous screening methods have been used to detect compounds that inhibit or stimulate T cell responses. One method screened for peptides or peptidomimetics that when bound to the peptide-binding groove of empty MHC molecules could block the ability of the MHC molecule to present other peptide antigens (See Lamont A. G. et al. (1990) J. immunol. 144:2493-2498, Falcioni F. et al. (1999) Nature Biotech. 17:562-567). As disclosed, this method identified compounds (i.e. MHC antagonists) that block the formation of a functional MHC antigen molecule. Another method screened for peptides that when bound to the peptide-binding groove of empty MHC molecules resulted in formation of MHC-peptide complexes that could act as TCR antagonists or agonists (See De Magistris M. T. et al. (1992) Cell 68:625-634, Sette A. et al. (1994) Annu. Rev. Immunol. 12:413-413, Wilson D. B. et al. (1999) J. Immunol. 163:6424-6434, Hiemstra, H. S. et al. (2000) Curr. Opin. Immunol. 12:80-84. Abrams S. I. et al. (2000) Curr. Opin. Immunol. 12:85-91). As reported, this method identified compounds that act in the context of the MHC as peptide antigens and antigen mimics (agonists) or as so-called altered peptide ligands (antagonists).

The present invention features screening methods that differ from those described above including the particular screens described in the immediately proceeding paragraph. As an illustration, the invention features methods for detecting and identifying compounds that modulate the interactions between the TCR and its cognate MHC antigen, including (pre-formed) cognate peptide-MHC complexes. The methods of the invention are not constrained to identifying compounds that bind to the peptide-binding groove of empty MHC molecules. Thus, the methods of the invention offer advantages in detecting a broad variety (or range) of compounds that modulate interactions between the TCR and the MHC antigen molecule. In addition, the present invention provides for particular advantages in detecting compounds that interact with TCR and/or MHC antigen molecules. As taught, the compounds identified by the methods of the invention would be expected to have different properties (i.e., binding specificities, composition, pharmacology) than those identified by the methods described above.

It is emphasized that notwithstanding the specific examples discussed above, the present invention is highly flexible and amenable to use of one or a combination of different screening formats including use of fully soluble molecules, cell based formats; and combinations thereof (e.g., one fully soluble molecule and one cell expressed molecule), and to any disease for which TCR:MHC antigen interactions play a role.

For many applications of the invention, components of specific screens will include soluble single chain TCR, soluble single chain MHC with or without genetically linked peptide or superantigen, a library of small molecules or other potential agonist or antagonist agents, and other compounds which are necessary to generate a detectable signal. The antagonist compounds will be screened to identify those that specifically block the interaction of the TCR with the MHC antigen. In the case where the MHC antigen is a peptide-MHC complex, screening will identify antagonist compounds that block TCR interactions with peptide presented by the MHC molecule as well as those that generally inhibit interaction with the MHC component or a specific MHC family. In the case where the MHC antigen is a superantigen-MHC complex, screening will identify antagonist compounds that block TCR interactions with superantigen bound by the MHC molecule as well as those that generally inhibit interaction with the MHC component. In the case where the MHC antigen is a alloreactive (xenoreactive) MHC molecule, screening will identify antagonist compounds that block TCR interactions with the alloreactive (xenoreactive) MHC component.

In this embodiment, the present methods can provide additional advantages. As discussed, the screen can efficiently select for test compounds that specifically inhibit the interaction of a TCR with a MHC molecule bearing a unique peptide or those that inhibit the interaction with a particular MHC family. This compound could then be used to suppress autoimmune diseases or allergic conditions triggered by the interaction of the T cell (bearing the TCR) with the antigen presenting cell (bearing the peptide associated with the MHC). These inhibitory compounds would offer a significant advantage over therapies which suppress the overall immune response by inhibiting the reaction specific for the disease while leaving the rest of the immune response intact.

In another embodiment, the screen can efficiently select for test compounds that specifically inhibit the interaction of a TCR with a MHC molecule bearing a superantigen. This compound could then be used to suppress illness and disease conditions triggered by the interaction of the T cell (bearing the TCR) with the antigen presenting cell (bearing the superantigen associated with the MHC). Again, these inhibitory compounds would offer a significant advantage over therapies which suppress the overall immune response by inhibiting the reaction specific for the disease while leaving the rest of the immune response intact.

In yet another embodiment, the screen can efficiently select for test compounds that specifically inhibit the interaction of a TCR with an alloreactive (xenoreactive) MHC molecule. This compound could then be used to suppress immune responses associated with tissue transplantation triggered by the interaction of the T cell (bearing the TCR) with the antigen presenting cell (bearing the alloreactive or xenoreactive MHC). Again, these inhibitory compounds would offer a significant advantage over therapies that suppress the overall immune response by inhibiting the reaction specific for the disease while leaving the rest of the immune response intact.

In other embodiments, agonist compounds will be screened to identify those that specifically augment the interaction of the TCR with the MHC antigen. In the case where the MHC antigen is a peptide-MHC complex, screening will identify agonist compounds which augment TCR interactions with peptide presented by the MHC molecule as well as those which generally augment interaction with the MHC component or a specific MHC family. In the case where the MHC antigen is a superantigen-MHC complex, screening will identify agonist compounds that augment TCR interactions with superantigen bound by the MHC molecule as well as those that generally augment interaction with the MHC component. In the case where the MHC antigen is an alloreactive MHC molecule, screening will identify agonist compounds that augment TCR interactions with the alloreactive MHC component.

In this embodiment, the present methods can provide additional advantages. As discussed, the screen can efficiently select for test compounds that specifically augment the interaction of a TCR with a MHC molecule bearing a unique peptide or those that augment the interaction with a particular MHC family. This compound could then be used to stimulate immune responses triggered by the interaction of the T cell (bearing the TCR) with the antigen presenting cell (bearing the peptide associated with the MHC). Such compounds could be useful in the treatment of infectious agents and cancer. These agonist compounds would offer a significant advantage by offering a unique method of stimulating the immune reaction specific for the disease. These compounds may be useful in conjunction with traditional immunostimulatory therapies, such as vaccination.

In other embodiments, the agonists compounds could be useful in the treatment of immune disorders such as autoimmune diseases and allergies. Use of such compounds may lead to immune deviation or induction of immune responses capable of down regulating or suppressing the harmful immune response.

As discussed, the TCR may be one of several forms of the soluble molecule depending, e.g., upon the conditions of the assay. The preceding discussion and the examples, which follow, provide for many of these soluble TCR molecules. For example, the molecule can be expressed as a three domain single chain form (scTCR), i.e. the alpha variable domain (Vα) is fused to the beta variable domain (Vβ) and beta constant domain (Vβ) via a 20 amino acid glycine-serine linker ([G₄S]₄). In its monomeric form the TCR can be expressed as a soluble single chain or fused to a kappa constant region. Multimeric complexes of the TCR can be produced in a number of ways; by genetic fusion to the Ig constant region (CH1, CH2, and CH3) to produce a dimer, by co-expression of the scTCR-IgG kappa chain and scTCR-IgG heavy chain in a cell to produce a tetrameric complex, as well as other methods described herein.

The screening method is compatible with other TCR molecules. For example, higher order multimers may be made by changing the IgG to IgA or IgM. Functional multimers may also be formed by chemically cross-linking the scTCR to soluble solid supports such as dendrimers. Description of the forms and other uses of the multimeric TCR molecules will be filed in another patent disclosure. Also see U.S. patent application Ser. No. 09/422,375. Alternate forms of the TCR molecule may also be produced which contain the MHC-binding regions of the co-receptors CD4 or CD8.

The MHC antigen molecule employed in the screen may include a genetically tethered (presenting) peptide and may be one of several forms of the soluble molecule depending upon the conditions of the assay. Description of the soluble forms of this particular MHC antigen molecule can be found, e.g., in the U.S. patent application Ser. No. 08/596,387 and the U.S. patent application Ser. No. 08/960,190. Multimeric forms of the MHC antigen, similar to those described for the TCR, may be produced for the assay if necessary.

As discussed, the present screening methods are flexible and can be tailored to detect, identify and select different test compounds depending, e.g., on the subject TCR/MHC antigen molecules and desired results.

For example, it will often be preferred to detect test compounds that behave as an antagonist of a particular immune complex. In this embodiment, the particular testing method employed will preferably show at least about a 10% decrease in the formation and/or stability of the subject immune complex relative to at least one of the suitable controls provided herein.

Additionally preferred test compounds are deemed capable of being an agonist of the immune complex if the testing method shows at least a 10% increase in the formation and/or stability of the immune complex relative to at least one of the suitable controls provided herein.

More specifically preferred test compounds are capable of inhibiting or eliminating formation of the subject immune complex. Further preferred test compounds are capable of increasing or decreasing the affinity constant [K_{A}] of the immune complex.

Methods for determining the affinity constant are known in the field and include conventional thermodynamic measurements. See, for example, Alam, S M et al. (1999) Immunity 10:227-237. More specifically, if [TCR] is the molar concentration of unbound TCR molecule, [MHC antigen] is the molar concentration of unbound MHC antigen molecule and [TCR/MHC antigen] is the molar concentration of the immune complex, then K_{A} can be readily determined by measuring the amount of immune complex at about equilibrium. In embodiments in which detection of antagonistic test compounds is indicated, that compound preferably shows at least about a 10%, preferably at least about 20%, more preferably at least about 50%, and still more preferably at least about 75% or 100% decrease in affinity constant relative to at least one of the suitable controls provided herein such as the K_{A} in the absence of the test compound.

However in other embodiments in which the detection method is optimized to screen for agonists of the subject immune complex, preferred test compounds will show about a 10%, preferably at least about 20%, more preferably at least about 50%, and still more preferably at least about 75% or 100% up to a 500% to about 1000% increase in affinity constant K_{A} relative to the control.

Also preferred are test compounds that are deemed to increase or decrease the rate of formation of the immune complex.

Rates of immune complex formation can be ascertained using conventional thermodynamic approaches. See, for example, Alam, S M et al. (1999) Immunity 10:227-237. In particular, the rate of immune complex formation [α]and the rate of dissociation [k_{-I}] can be readily ascertained using such approaches and related techniques. Generally, to determine the rates of formation and dissociation, formation of a subject immune complex between a particular TCR and MHC antigen molecule is monitored over a time period (e.g., a few seconds up to several minutes or more) in the presence and absence of the test compound. In embodiments in which the test compound is deemed to be an antagonist, that compound preferably shows at least about a 10%, preferably at least about 20%, more preferably at least about 50%, and still more preferably at least about 75% or 100% decrease in the rate of immune complex formation [α]relative to at least one of the suitable controls discussed herein, e.g., the rate of immune complex formation in the absence of the test compound.

Alternatively, in embodiments in which the test compound is deemed to destabilize the immune complex and enhance the rate of dissociation, that compound preferably shows about a 10%, preferably at least about 20%, more preferably at least about 50%, and still more preferably at least about 75% or 100% up to a 500% to about 1000% increase in rate of dissociation [k_{-I}] relative to the control.

In those embodiments in which the test compound enhances the rate of dissociation of the immune complex, that test compound may sometimes be referred to herein as destabilizing TCR/MHC antigen interactions and/or destabilizing the immune complex.

Also preferred are test compounds that show capacity to modulate immune complex avidity. Avidity can be observed and quantified, if desired, by one or a combination of immunological techniques such as those described below. In embodiments in which a particular test compound reduces the avidity of a TCR and MHC antigen molecules, that compound preferably shows at least about a 10%, preferably at least about 20%, more preferably at least about 50%, and still more preferably at least about 75% or 100% decrease in avidity relative to at least one of the suitable controls provided herein including such avidity in the absence of the test compound. In another embodiment however in which the test compound is deemed to be an agonist, that compound preferably shows about a 10%, preferably at least about 20%, more preferably at least about 50%, and still more preferably at least about 75% or 100% up to a 500% to about 1000% increase in avidity relative to the control.

Also preferred are test compounds that show capacity to modulate immune complex multimerization. Multimerization can be observed and quantified, if desired, by one or a combination of techniques. See, for example, Alam et al. (1999) Immunity 10:227-237, Reich et al. (1997) Nature 387:617-620. In embodiments in which a particular test compound reduces the multimerization of a TCR and MHC antigen molecules, that compound preferably shows at least about a 10%, preferably at least about 20%, more preferably at least about 50%, and still more preferably at least about 75% or 100% decrease in avidity relative to at least one of the suitable controls provided herein including such multimerization in the absence of the test compound. In another embodiment however in which the test compound is deemed to be an agonist, that compound preferably shows about a 10%, preferably at least about 20%, more preferably at least about 50%, and still more preferably at least about 75% or 100% up to a 500% to about 1000% increase in multimerization relative to the control.

Further preferred are test compounds that show capacity to modulate, i.e., increase or decrease at least one cell response, typically a T cell or T cell hybridoma response, by about 1.5 to about 100 times relative to a suitable control. In particular embodiments of the methods disclosed herein, a preferred response is cytokine production and particularly production of the IL-2 cytokine.

Additionally suitable test compounds of the invention preferably have negligible capacity to modulate formation of a control immune complex. In one embodiment, that control immune complex includes the TCR molecule and an antibody capable of specifically binding an epitope on the TCR molecule not specifically bound by a particular MHC antigen molecule such as a peptide bound MHC molecule (anti-TCR antibody). Alternatively, the control immune complex includes at least one cluster of differentiation (CD) protein associated with the TCR molecule, e.g., in a cell membrane. Preferably, the CD protein is CD3 and the antibody is capable of specifically binding CD3 associated with the TCR molecule (anti-CD3 antibody).

As discussed, it is an object of this invention to provide useful pharmaceutical compositions that include at least one of the test compounds detected by the present methods. Such compositions can be administered to a subject in any of several ways. For example, a given test compounds or combination of test compounds can be administered as a prophylactic to prevent the onset of or reduce the severity of a targeted immune condition. Alternatively, such test compounds or pharmaceutical compositions can be administered before, during or after the course of the targeted condition.

A pharmaceutical compound of this invention, sometimes referred to as a treatment compound, can be administered to a subject, either alone or in combination with one or more therapeutic agents, as a pharmaceutical composition in mixture with conventional excipient, i.e. pharmaceutically acceptable organic or inorganic carrier substances suitable for parenteral, enteral or intranasal application which do not deleteriously react with the active compounds and are not deleterious to the recipient thereof. Suitable pharmaceutically acceptable carriers include but are not limited to water, salt solutions, alcohol, vegetable oils, polyethylene glycols, gelatin, lactose, amylose, magnesium stearate, talc, silicic acid, viscous paraffin, perfume oil, fatty acid monoglycerides and diglycerides, petroethral fatty acid esters, hydroxymethyl-cellulose, polyvinylpyrrolidone, etc. The pharmaceutical preparations can be sterilized and if desired mixed with auxiliary agents, e.g., lubricants, preservatives, stabilizers, wetting agents, emulsifiers, salts for influencing osmotic pressure, buffers, colorings, flavorings and/or aromatic substances and the like which do not deleteriously react with the active compounds.

Such compositions may be prepared for use in parenteral administration, particularly in the form of liquid solutions or suspensions; for oral administration, particularly in the form of tablets or capsules; intranasally, particularly in the form of powders, nasal drops, or aerosols; vaginally; topically e.g. in the form of a cream; rectally e.g. as a suppository; etc.

The pharmaceutical agents may be conveniently administered in unit dosage form and may be prepared by any of the methods well known in the pharmaceutical arts, e.g., as described in Remington 's Pharmaceutical Sciences (Mack Pub. Co., Easton, Pa., 1980). Formulations for parenteral administration may contain common excipients such as sterile water or saline, polyalkylene glycols such as polyethylene glycol, oils of vegetable origin, hydrogenated naphthalenes and the like. In particular, biocompatible, biodegradable lactide polymer, lactide/glycolide copolymer, or polyoxyethylene-polyoxypropylene copolymers may be useful excipients to control the release of certain test compounds identified by the methods of this invention.

Other potentially useful parenteral delivery systems include ethylene-vinyl acetate copolymer particles, osmotic pumps, implantable infusion systems, and liposomes. Formulations for inhalation administration contain as excipients, for example, lactose, or may be aqueous solutions containing, for example, polyoxyethylene-9-lauryl ether, glycocholate and deoxycholate, or oily solutions for administration in the form of nasal drops, or as a gel to be applied intranasally. Formulations for parenteral administration may also include glycocholate for buccal administration, methoxysalicylate for rectal administration, or citric acid for vaginal administration. Other delivery systems will administer the therapeutic agent(s) directly at a body site of interest

A pharmaceutical composition of this invention can be employed in the present treatment methods as the sole active pharmaceutical agent or can be used in combination with other active ingredients including known agents with recognized immunomodulatory effects. Examples of such agents include, but are not limited to, cyclosporin, certain cytotoxic agents, lymphocyte immune globulin, adrenocorticosteroids, sulfasalazine, FK-506, methoxsalen, rapamycin, and thalidomide. For disclosure relating to these and other useful agents see, e.g., The Pharmacological Basis of Therapeutics (8th ed.) Gilman, A. et al. (eds.) McGraw-Hill Health Professionals Division, pp. 1264-1276, (1993).

The concentration of one or more treatment compounds in a therapeutic composition will vary depending upon a number of factors, including the dosage of the particular formulation to be administered, the chemical characteristics (e.g., hydrophobicity) of the composition employed, and the intended mode and route of administration. In general terms, one or more than one of the detected test compounds may be provided in an aqueous physiological buffer solution containing about 0.1 to 10% w/v of a compound for parenteral administration.

It will be appreciated that the actual preferred amounts of active compounds used in a given therapy will vary according to e.g. the specific compound being utilized, the particular composition formulated, the mode of administration and characteristics of the subject, e.g. the species, sex, weight, general health and age of the subject. Optimal administration rates for a given protocol of administration can be readily ascertained by those skilled in the art using conventional dosage determination tests conducted with regard to the foregoing guidelines. Suitable dose ranges may include from about 1 μg/kg to about 100 mg/kg of body weight per day.

Therapeutic compounds of the invention are suitably administered in a protonated and water-soluble form, e.g., as a pharmaceutically acceptable salt, typically an acid addition salt such as an inorganic acid addition salt, e.g., a hydrochloride, sulfate, or phosphate salt, or as an organic acid addition salt such as an acetate, maleate, fumarate, tartrate, or citrate salt. Pharmaceutically acceptable salts of therapeutic compounds of the invention also can include metal salts, particularly alkali metal salts such as a sodium salt or potassium salt; alkaline earth metal salts such as a magnesium or calcium salt; ammonium salts such an ammonium or tetramethyl ammonium salt; or an amino acid addition salts such as a lysine, glycine, or phenylalanine salt.

For many applications of this invention, it will be helpful to use TCR and MHC antigen molecules that are substantially pure particularly in embodiments in which fully soluble molecules are employed. Preferably, such molecules will have been isolated from cell substituents that naturally accompany it so that the fusion proteins are present in at least 90 to 95% homogeneity (w/w). TCR and MHC antigen molecules including recombinant fusion proteins having at least 98 to 99% homogeneity (w/w) are most preferred for many applications of this invention such as those involving pharmaceutical, clinical and research use. Once substantially purified the molecules should be substantially free of contaminants for such applications. Once purified partially or to substantial purity, the molecules can be used therapeutically, or in performing in vitro or in vivo assays as disclosed herein. Substantial purity can be determined by a variety of standard techniques such as chromatography and gel electrophoresis. See generally Sambrook et al. in Molecular Cloning: A Laboratory Manual (2d ed. 1989); and Ausubel et al. (1989), Current Protocols in Molecular Biology, John Wiley & Sons, New York for discussion relating to many of these standard methods; the disclosures of which are incorporated herein by reference.

As discussed, the TCR and MHC antigen molecules can be separated and purified by appropriate combination of known techniques. These methods include, for example, methods utilizing solubility such as salt precipitation and solvent precipitation, methods utilizing the difference in molecular weight such as dialysis, ultra-filtration, gel-filtration, and SDS-polyacrylamide gel electrophoresis, methods utilizing a difference in electrical charge such as ion-exchange column chromatography, methods utilizing specific affinity such as affinity chromatography, methods utilizing a difference in hydrophobicity such as reverse-phase high performance liquid chromatography and methods utilizing a difference in isoelectric point, such as isoelectric focusing electrophoresis, metal affinity columns such as Ni-NTA. See generally Sambrook et al. and Ausubel et al. supra for disclosure relating to these methods.

By the term “fully soluble” or similar term is meant that the subject TCR or MHC antigen molecule is not readily sedimented under low G-force centrifugation (e.g. less than about 30,000 revolutions per minute in a standard centrifuge) from an aqueous buffer, e.g., cell media. Further, the fusion molecule is soluble if the it remains in aqueous solution at a temperature greater than about 5-37° C. and at or near neutral pH in the presence of low or no concentration of an anionic or non-ionic detergent. Under these conditions, a soluble protein will often have a low sedimentation value e.g., less than about 10 to 50 svedberg units.

As used herein, the terms “fragment” or “functional fragment” as used in reference to a TCR molecule means a portion of that molecule (heterodimer or single chain construct comprising essentially full-length V chains) that is capable of specifically binding at least about 70% and preferably at least about 80%, 90%, 95% up to 100% or more of a particular MHC antigen molecule when compared to the corresponding full-length molecule. As an illustration, a full-length sc-TCR is defined as a molecule with a full-length V-α and V-β chain. Similarly, a full-length TCR heterodimer is defined as a molecule having full-length α and β chains particularly those also with corresponding transmembrane and cytoplasmic domains.

As used in reference to a MHC antigen molecule as disclosed herein, the term “fragment” or “functional fragment” means a portion of that molecule (heterodimer or single chain construct comprising essentially full-length chains) that is capable of specifically binding at least about n 70% and preferably at least about 80%, 90%, 95% up to 100% or more of a particular TCR molecule when compared to the corresponding full-length molecule. As an illustration, a full-length sc-NMC is defined as a molecule with a full-length α and β chain. Similarly, a full-length MHC or HLA heterodimer is defined as a molecule having full-length α and β chains particularly those also with corresponding transmembrane and cytoplasmic domains.

The terms “fragment” or “functional fragment” as used to reference a particular CD3ζ sequence is meant a portion of the full-length sequence capable of inducing a cellular response of this invention. Preferred are fragments capable of binding at least about 70% and preferably at least about 80%, 90%, 95% up to 100% or more of a particular cellular response when compared to the corresponding full-length molecule.

Assays for detecting specific binding of the fragments and functional fragments disclosed are discussed herein and specifically include flow cytometry and BIAcore assays.

### EXAMPLE 1

### Generation of Monovalent Single-Chain MHC/Peptide Complexes

A. Construction of Vectors for Expressing Soluble Single-Chain IE^{k} MHC Class II Molecules with a Specific Presenting Peptide.

The goal of these studies was to develop screening methods for detecting agonists and antagonists of interaction between the MHC antigen molecule, such as an MHC/peptide complex, and the T cell receptor (TCR). It has been demonstrated previously that the MHC class IA^{d}/peptide complex could be generated as a recombinant single-chain molecule that retains its ability to interact with TCR and stimulate T cell responses (Rhode et al. 1996. J. Immunol. 157: 4885, WO 99/21572, U.S. Ser. No. 09/204,979). The generation of single-chain (sc) murine IA^{d}/peptide and human HLA-DR2/peptide complexes has been described (WO 99/21572, U.S. Ser. No. 09/204,979). The sc-class II molecules consists of a fusion between the β1-β2 fragment and the α1-α2 fragment via a flexible peptide linker. In addition, antigenic peptide sequences were linked to the amino terminus of the β1 domain via a peptide linker. For example, sequences encoding the chicken ovalbumin peptide, OVA 323-339 (ISQAVHAAHAEINEAGR) or the HSV-1 glycoprotein D peptide, GD 246-261 (APYTSTLLPPELSETP) were linked to the β1 gene fragment of the scIA^{d} gene fusion to create the scIA^{d}/OVA and scIA^{d}/GD molecules, respectively. The antigenic peptides were positioned such that they could bind to the peptide-binding groove formed by the properly folded α1 and β1 domains. These molecules were shown to be capable of specifically stimulating T cell responses in vitro. For example, the scIA^{d}/OVA protein when immobilized on a tissue culture plate was capable of stimulating IL-2 release from the DO11.10 T cell hybridoma whereas the scIA^{d}/GD protein stimulated IL-2 release from the GD12 T cell hybridoma (Rhode et al. 1996. J. Immunol. 157: 4885). As described in the example below, these T cell assays were modified to identify compounds that inhibited the sc-MHC/peptide molecule's ability to stimulate T cell responses. As a control for the MHC specificity of these inhibitors, a T cell assay involving specific stimulation using a different murine sc-class II/peptide complex was developed. Generation of this murine sc-class II/peptide molecule, single-chain IE^{k} linked to PCC 88-104 (scIE^{k}/PCC), is described as follows.

The MHC class II genes used for the scIE^{k}/PCC constructs were isolated by PCR amplification of cDNA generated from the appropriate APC, CH12 cells. Modified versions of gene fragments of the IE^{k} α and β chains were generated by PCR amplification using the cloned genes as template DNA. These fragments were assembled in the cloning scheme shown in FIG. 7A-C of the drawings and resulted in a chimeric gene encoding the antigenic peptide, PCC 88-104, linked to a single-chain IE^{k} molecule.

The development of the various genetic constructs containing individual α and β chain fragments is described in the following paragraph. The α1-α2 gene fragment first strand cDNA served as the template for PCR amplification using primers VW487 (470) and VW489 (477). The sequences of the oligonucleotides used in cloning are shown in Table 1. This fragment was extended at the 3′ end by reamplifying with VW487 (470) and VW485 (471) which resulted in the α fragment ending with the IA^{d} transmembrane “hinge” region followed by the amino acid sequence MSGGGGC. This fragment was cloned into pGEM T-Easy (Clonetech, Palo Alto, Calif.) to yield plasmid pTVW406. Subsequent amplification of this α chain insert with primers VW487 (470) and VW490 yielded a fragment flanked by XhoI and EcORI containing a “birA” site for in vitro biotinylation at the 3′ end. This fragment was cloned into pGEM T-Easy to yield plasmid pTVW420-8. Both the C terminal and the BirA terminal fragments contain a XhoI restriction site at the 5′ end and an EcORI restriction site at the 3′ end. The β₁-β₂ gene fragment first strand cDNA served as the template for PCR amplification using primers VW486 (469) and VW488 (472). This fragment was extended at the 5′ end by reamplifying with VW484 (468) and VW486 (469), which resulted in the p fragment beginning with LSSAS where the codons for LS represents an AflII restriction site and those for AS represents a NheI restriction site. The fragment was cloned into pGEM T-Easy (Clonetech, Palo Alto, Calif.) to yield plasmids pTVW407-1 and-3. The resulting construct had 13 amino acids preceding the first amino acid of the mature β chain, LS (AflII) S AS (NheI) GGGGSGGG. Oligonucleotide primers, VW482 (466) and VW483 (467), encoding the PCC peptide (KAERADLIAYLKQATAK) and overhanging cohesive ends which match those left by digestion with AflII and NheI, were annealed and cloned into pTVW407-3 which had been digested with AflII+NheI to yield plasmid pTVW410-35.

A description of the construction of the scIE^{k} with follows. A pGEM T-Easy vector containing the scIA^{d}/OVA fusion gene was constructed to serve as a cloning template for the scIE^{k} gene. The NcoI to EcORI fragment from plasmid SSC1 (see WO 99/21572, U.S. Ser. No. 09/204,979) containing the scIA^{d}/OVA fusion gene was introduced into a pGEM T-Easy based vector (pTVW406) cut with the same restriction enzymes. This resulting cloning template (pTVW408) contains the β chain leader sequence, the β chain with linked peptide flanked by the restriction sites AflII+SpeI, a 20 amino acid linker sequence (G₄S)₄, and the α chain flanked by restriction sites XhoI and EcORI. Alpha chain sequence containing a carboxyl terminal C derived from pVW406-2 was introduced into pTVW408 as a restriction fragment bounded by XhoI and EcORI resulting in a vector, 406+408, that contained the IA^{d} β°chain and the IE^{k}α chain. The IE^{k}β chain fragments containing either the linked PCC peptide or a single serine (derived from pTVW407-3 and 410-35, respectively) were cloned into this plasmid backbone after digestion with AflII and SpeI resulting in plasmids pTVW417-6 and pTVW416-6, respectively. The β-chain in these constructs contained a mutation that resulted in amino acid 38 being changed from an asparagine (N) to a serine (S). This was corrected by replacing a HindIII to SpeI fragment in pTVW417-6 and pTVW416-6 with one from pTVW407-1 (which contained the correct p chain sequence) to yield plasmids pTVW422-1 (containing PCC peptide) and pTVW423-2 (containing serine). The resulting scIE^{k} gene fusions were cloned back into plasmid SSC 1 as AflII/EcORI -fragments resulting in plasmids pTVW424-1 (PCC) and pTVW425-2 (S). This was done in order to incorporate sequences important for efficient translation (i.e. the Kozak sequence). The sequence encoding the birA site was added to the single chain construct by cloning a XhoI to EcORI fragment from pTVW420-8 into pTVW423-2 resulting in plasmid pTVW427-3.

Insect cell expression vectors containing the scIek genes were constructed in plasmid pBluBac4.5 (InVitrogen) such that they encoded each of the following combinations of β and α chain; β preceded by the PCC peptide linked to α with a carboyl-terminal cysteine (PCC/C), β preceded by serine (“blank” form) linked to α with a carboxyl-terminal cysteine (S/C), β preceded by the PCC peptide linked to α with a carboxyl-terminal birA sequence (PCC/birA), and β preceded by serine (“blank” form) linked to a with a carboxyl-terminal birA sequence (S/birA). The PCC/C and S/C forms of the vectors were constructed by cloning XmaI to EcORI fragments from pTVW424-1 and pTVW425-2, respectively, into pBlueBac4.5 cut with the same enzymes yielding plasmids pTVW429-2 and pTVW428-1, respectively. The birA forms of these vectors were made by cloning a SpeI to EcORI fragment from pTVW427-3 into pTVW429-2 and pTVW428-1 to yield pTVW431-1 (PCC/birA) and pTVW430-1 (S/birA).

TABLE 1

Oligonucleotides used in cloning

| | |
|---|---|
| Designation | Sequence |

| | |
|---|---|
| VW482 (466) | 5′-tta agt aag gca gaa cga gca gat cta ata gct |
| | tat cta aaa aaa gcc acc gcg aag g-3′ |
| VW483 (467) | 5′-cta gcc ttc gcg gtg gct tgt ttt aga taa gct |
| | att aga tct gct cgt tct gcc tta c-3′ |
| VW484 (468) | 5′-ctt aag tag cgc tag cgg agg ggg cgg aag cgg |
| | cgg agg ggc aag ctt cag acc atg g-3′ |
| VW485 (471) | 5′-ccc gaa ttc tat tag cag cct cct cca cct gac |
| | att ggg agg agg gtt ttc tc-3′ |
| VW486 (469) | 5′-cca cta gtc cac tcg acc gtg aca gg-3′ |
| VW487 (470) | 5′-ggt tcc tcg agt atc aaa gag gaa cac acc atc-3′ |
| VW488 (472) | 5′-gga ggg gca agc ttc aga cca tgg-3′ |
| VW489 (477) | 5′-tga cat tgg gag gag ggt ttt ctc ttc-3′ |
| VW490 | 5′-gta cga att cta tta ttc gtg cca ttc gat ttt |
| | ctg agc ttc gaa gat gtc gtt cag gcc tcc tcc acc |
| | tga cat-3′ |

B. Production of Soluble scIE^{k}/PCC Molecules in Insect Cells.

Purified plasmids pTVW429-2, pTVW430-1 and pTVW401-l were used in cotransfection with Bac-N-Blue DNA (InVitrogen) and recombinant virus was isolated and enriched as described in Invitrogen's Bac-N-Blue Transfection Kit Manual, Version F. SF9 cells were used throughout.

Supernatant samples from plates or production flasks were tested for the presence of recombinant product (scIE^{k}) by ELISA. The ELISA involves coating 100 ng of 14-4-4S (PharMingen) anti-IE^{k} antibody onto wells of Nunc Maxisorp plates in 100 μl of PBS (phosphate buffered saline) overnight at 4° C. Blocking was done with 200 μl 10% FBS (fetal bovine serum)-PBS for at least one hour at 37 C and plates were washed three times with wash buffer (Tris-buffered saline (TBS)-0.1% Tween 20). Samples were added at 100 1/well and incubated 1 hr at room temperature. Plates were then washed 4 times with 200 μl wash buffer and 50 ng of biotinylated antibody 17-7-3 (PharMingen) was added in 100 μl 10% FBS-PBS followed by incubation for 1 hour at room temperature. Plates were washed 5 times with wash buffer and 100 μl of a 100 ng/ml NeutrAvidin horseradish peroxidase conjugated (Pierce) was added. After incubation for one hour at room temperature the plates were washed 5 times with wash buffer and 100 μl of using 3,3′,5,5′-tetramethylbenzidine (TMB) reagent (BioFX Laboratories) was added per well. One hundred 1 of 0.18M H₂SO₄ was added to quench the reaction and absorbance was read at 450 mm.

The cotransfection mixes were plated for individual plaques as outlined in the InVitrogen manual. Isolated blue plaques were used to prepare small-scale plate lysates to be used as viral stocks and to be tested for IE^{k} reactivity by the ELISA described above. An example of A₄₅₀ values for supernatants from 5 plaques resulting from a cotransfection with pTVW431-1 (PCC/birA) is shown in Table 2. The specificity of the signal is due to the secretion of scIE^{k}/PCC into culture supernatant by SF9 cells.

TABLE 2
IE^{k} ELISA of insect cell culture supernatant from cotransfection

| | A₄₅₀ values Plaque Designation | | | | |
|---|---|---|---|---|---|
| μl supernatant | A | B | C | D | E |
| 100 | 0.495 | 0.432 | 0.235 | 0.427 | 0.292 |
| 50 | 0.276 | 0.243 | 0.146 | 0.280 | 0.177 |
| 25 | 0.162 | 0.162 | 0.110 | 0.189 | 0.127 |
| 0 | 0.052 | 0.051 | 0.055 | 0.053 | 0.055 |

High titer virus stocks were prepared from the small-scale plate lysates and used to infect 1-liter cultures of SF9 cells grown to 1×10⁶ cells/ml. Supernatant from these infections was used to purify the scIE^{k} as described below. The purified scIE^{k} with linked PCC peptide was assayed for its ability to modulate the activation of the 2B4 T cell hybridoma as described below.

C. Purification of scIE^{k}/PCC Molecules Expressed in Insect Cells.

Supernatant from SF9 cells infected with recombinant baculovirus expressing sc IE^{k}/PCC protein was harvested 5 days after infection. Cells were removed from the medium by centrifugation at 9,500×g, 4° C. for 10 minutes. The pH of the clarified supernatant was then adjusted to 8 resulting in a cloudy white precipitate. This precipitate was removed by centrifugation at 12,000×g, 4° C. for 30 min to 2 hours. The supernatant from this centrifugation was filtered through a 0.2μ filter in preparation for immunoaffinity chromatography.

Immunoaffinity chromatography was carried out by applying the sample prepared as above on to a column of Sepharose 4B (Pharmacia) cross-linked with N22, an anti-IA and IE antibody produced by ATCC (American Type Culture Collection) cell line HB225. The column was equilibrated with 20 mM Tris pH 8.0 and was washed with this buffer until the A₂₈₀ₙₘ baseline was reached. The antibody column was then washed with the same buffer containing 1 M NaCl to remove nonspecifically bound proteins. The IE^{k} protein was eluted by application of PBS followed by 50 mM glycine-NaOH, pH 11. The protein eluted in two peaks (monitored by A₂₈₀), the first in the PBS and the second in pH 11. The peak eluted at pH 11 was immediately adjusted to pH 7-8 with HCl. The samples from each peak were then concentrated and buffer exchanged into PBS by ultrafiltration. The purity and functionality of lEk samples were tested by sodium dodecyl sulfate-polyacrylamide gel electrophoresis and T cell activation assay (see below), respectively. Purified scIE^{k}/PCC from this preparation showed no contaminating bands on a Coomassie stained polyacrylamide gel and a total protein concentration of 0.37 mg/ml as assayed by absorbance.

D. Activation of T Cells by scIE^{k}/PCC Complex.

The T cell hybridoma 2B4 responds to the PCC peptide in the context of IE^{k} class II MHC molecules by producing IL-2. The production of IL-2 was assessed in an activation assay performed as described in the following paragraph. Individual wells in Nunc Maxisorp plates were coated with a mixture of scIE^{k}/PCC and anti-murine IL-2 antibody (PharMingen, 18161D) suspended in 100 μl PBS such that the scIE^{k} molecule was present at concentrations between 400 and 12.5 ng/well and the anti-IL2 antibody was present at 100 ng/well. After coating overnight at 4° C. the solution was removed from the plate and 1×10⁵ 2B4 cells were added per well in 100 μl of IMDM containing 10% FBS. The plate was incubated for 8 hours at 37° C. in 10% CO₂ and then washed three times with wash buffer (TBS with 0.1% Tween-20). Biotin-labeled anti-murine IL-2 antibody (PharMingen, 18172D) in 10% FBS-PBS was added and incubated overnight at 4° C. Plates were then washed 3 times with wash buffer and 100 μl of a 100 ng/ml NeutrAvidin horseradish peroxidase conjugated (Pierce) was added and incubated for 15 min. to one hour at room temperature. This incubation was followed by 3 washes with wash buffer and 100 μl of TMB substrate was added and allowed to develop. One hundred μl of 0.18M H₂SO₄ was added to quench the reaction and absorbance was read at 450 nm. Results of one assay demonstrating that the scIE^{k}/PCC activates 2B4 cells are shown in Table 3. Peak stimulation took place between 100 and 200 ng of scIE^{k}/PCC fixed on the plate and absorbance values decreased at higher and lower concentrations.

TABLE 3
Activation of 2B4 T cells by scIE^{k}/PCC protein

| scIE^{k}/PCC (ng/well) | Absorbance (450 nm) |
|---|---|
| 400 | 0.216 |
| 200 | 0.485 |
| 100 | 0.413 |
| 50 | 0.234 |
| 25 | 0.081 |
| 12.5 | 0.054 |
| 0 | 0.056 |

### EXAMPLE 2

### Generation of Multi-Valent Single-Chain IA^{d}/Peptide-Biotin/Avidin Complexes

A. Construction of Vectors for Expressing scIA^{d}/Peptide-BirA Complexes.

Example 1 describes the generation and use of monovalent sc-class II/peptide molecules in stimulating T cell responses. One of the limitation of monovalent complexes as probes for MHC antigen-TCR interactions is the intrinsic low affinity between the MHC antigen molecule and the TCR. This problem can be overcome through the formation of multi-valent MHC antigen complexes. These multi-valent complexes have a number of uses in developing screening methods (described in the examples below) for detecting inhibitors of the MHC antigen-TCR interaction. Described in this example and in examples 3 to 5 are methods for generating multi-valent single-chain MHC antigen complexes.

One method of generating multi-valent MHC antigens such as multi-valent MHC/peptide complexes involves biotinylating the MHC/peptide molecule and binding up to four of the biotinylated molecules to a single avidin molecule. Specific peptide sequences that serve as target sequences for the biotin ligase enzyme can be added to the MHC/peptide molecule. These sequences allow the enzymatic addition of a single biotin moiety per MHC/peptide molecule. The biotinylated MHC/peptide molecules can then form multi-valent complexes (dimers to tetramers) via high affinity interactions between the biotin and avidin.

For example, a DNA sequence encoding the IA^{d} α1-α2 fragment tagged with the biotin ligase BirA target sequence, GGGSLNDIFEAQKIEWHE, was transferred from pTVW 421-41 as a XhoI-EcORI fragment into pTVW408-1 to create pMBBirI. This vector was then cut with SalI and the restriction site overhang was digested to create a blunt end. The vector was then cut with NcoI and the DNA insert fragment was purified. The Blubac III vector (InVitrogen part number 120415) was cut with Hind III and the restriction site overhang was digested to create a blunt end. The vector was then cut with NcoI and the vector backbone fragment was purified and ligated to the insert fragment. The resulting Blubac III vector, pMB033, carries the scIA^{d}/OVA-BirA insert.

B. Production of Soluble scIA^{d}/OVA-BirA Molecules in Insect Cells.

The Blubac III vector carrying the scIA^{d}/OVA-BirA insert was used in a cotransfection and recombinant virus was enriched from wild type AcMNPv by plaque purification (see D. R. O'Reilly et al. Baculovirus expression vectors: A laboratory manual. W. H. Freeman & Co. New York (1992) and the InVitrogen Bac-N-Blue Transfection Kit Instruction Manual Version G Catalog # K855-01). SF9 cells were used throughout.

Insect cells were infected at high multiplicity of infection (moi) in TNMFH insect cell media (JRH Biosciences part no. 51942-79P) with 10% fetal bovine serum (Hyclone SH 30071.03) to produce recombinant scIA^{d}/OVA-BirA protein. Approximately 96 hours post infection, the pH of the infection supernatant was adjusted to 8.0 with 10N NaOH, centrifuged at high speed (greater then 10,000×g) to remove particulate matter and 0.2 μm filtered. The processed supernatant was tested by an IA-specific ELISA as described previously (Rhode et al. 1996. J. Immunol. 157: 4885).

C. Purification and Multimerization of scIA^{d}/OVA-BirA Molecules.

Filtered infection supernatant at pH 8.0 was applied onto a column of Protein A Sepharose cross-linked with M5, an anti-IA^{d} monoclonal antibody. Following sample application, the immunoaffinity column was washed with 20 mM Tris-HCl, pH 8.0 until A₂₈₀ₙₘ baseline was reached. The antibody column was then washed with the same buffer containing 1M NaCl as above to remove non-specific bound proteins. The scIA^{d}/OVA-BirA protein was then eluted with 50 mM glycine NaOH, pH 11. The eluted peak protein (monitored by A₂₈0 nm) was immediately adjusted to pH 8.0 with 2M glycine, pH 2.0, concentrated and buffer exchanged into PBS by ultrafiltration. The purified sample was stored at 4-8° C. The purity and functionality of the scIA^{d}/OVA-BirA sample were tested by sodium dodecyl sulfate-polyacrylamide gel electrophoresis (SDS-PAGE).

Biotinylation of the lysine in the BirA tag was achieved by addition of E. coli Biotin Ligase enzyme (Avidity BIRA500) and Biomix buffers A and B (Avidity, supplied with Biotin Ligase). Biotinylated protein was incubated with avidin to form complexes of the scIA^{d}/OVA-BirA. At a 4:1 ratio of scIA^{d}/OVA-BirA:Avidin, about 80% of the scIA^{d}/OVA-BirA molecules formed multimeric complexes larger than monomer as seen by SDS-PAGE.

D. Activation of T Cells by Multi-Valent scIA^{d}/OVA-BirA Complexes.

To test the activity of these multimers, either multimer or monomer (no avidin) was coated on Nunc microtiter wells, DO11.10 cells were added and after 24 hours supernatant was harvested and relative IL-2 levels were determined by ELISA as described previously (Rhode et al. 1996. J. Immunol. 157: 4885). Absorbance values for a typical experiment are shown in Table 4.

TABLE 4
Activation of DO11.10 T cells by
multi-valent scIA^{d}/OVA-BirA complexes

| | IL-2 ELISA Values (A₄₅₀) | |
|---|---|---|
| | Stimulation by | |
| ng complex/ | multi-valent scIA^{d}/OVA- | Stimulation by monovalent |
| well | BirA complexes | scIA^{d}/OVA-BirA complexes |
| 1000 | 1.865 | 1.164 |
| 500 | 1.598 | 1.055 |
| 125 | 0.916 | 0.513 |
| 62 | 0.744 | 0.398 |
| 31 | 0.554 | 0.242 |

It was shown that the scIA^{d}/OVA-BirA molecule could be biotinylated, that a single molecule of avidin could bind more than one molecule of biotinylated scIA^{d}/OVA-BirA and that multimer was a much more potent stimulator of IL-2 production than monomer.

### EXAMPLE 3

### Generation of Multi-Valent Single-Chain IA^{d}/Peptide-IgG Complexes

A. Construction of Vectors for Expressing scIA^{d}/Peptide-IgG Complexes.

A second strategy to create multi-valent MHC antigen complexes made use of the immunoglobulin constant domains as a molecular scaffold. In this approach, expression vectors were constructed encoding single-chain MHC/peptide molecules as fusion to the constant domains of IgG heavy and light chains. The generation of DNA constructs expressing peptide-linked single-chain MHC class II molecules fused to immunoglobulin Cκ domains has been described previously (WO 99/21572). The pIADK vector contains an expression cassette encoding the OVA 323-339 peptide linked to an scIA^{d} fused to the mouse IgG Cκ domain. The pDRHK vector contains an expression cassette encoding the human MBP 84-102 peptide linked to an scDR2 (1501) fused to the human IgG Cκ domain. Assembly of these vectors was described in detail previously (WO 99/21572). These vectors have been deposited with the ATCC pursuant to the Budapest Treaty. The DNA vectors were deposited on Sep. 17, 1997 and have been assigned Accession Nos. 209274 (pDRHK) and 209275 (pIADK). These vectors and their construction intermediates served as starting materials for the construction of the vectors described below and in Example 4.

In addition, a vector was constructed for the mammalian expression of the scIA^{d}/GD-mouse IgG Cκ fusion. To construct this vector, a bacterial shuttle vector pJA (see WO 99/21571) containing the scIA^{d}/OVA construct was digested with EcORV and NheI to remove sequence encoding the OVA peptide. Annealed oligonucleotides (OPR225/OPR226-oligonucleotides are listed in Table 5) encoding the gD 246-261 peptide were subcloned into the digested pJA vector, resulting in the pIAGK shuttle vector. The pIAGK vector was then digested with EcORV and BstBI. The fragment containing the scIA^{d}/GD gene was purified and subcloned into EcORV/BstBI-digested pIADK vector, resulting in the pJAGMK expression vector. This vector is capable of expressing the scIA^{d}/GD-Cκ fusion in mammalian cells.

Vectors capable of expressing fusions between the scIA^{d}/peptide molecules and the C_{H}1-C_{H}2-C_{H}3 domains of murine IgG2b heavy chain have been constructed. For the scIA^{d}/OVA construct, pIADK served as template DNA for PCR amplifications using oligonucleotide primers OPR237 and OPR239. For the scIA^{d}/GD construct, pIAGMK served as template DNA for PCR amplifications using oligonucleotide primers OPR238 and OPR239. The resulting PCR product carries the scIA^{d}/OVA or scIA^{d}/GD gene fusions carrying a 5′ NruI site and a 3′ EcORI site needed for cloning into the mammalian expression vector, pSUN7. This pcDNA3-based vector carries an expression cassette encoding immunoglobulin leader sequence and the mouse IgG2b C_{H}2-C_{H}2-C_{H}3 domains. In this vector, NruI and EcORI restriction sites are located near the 3′ end of the leader sequence and the beginning (5′ end) of the CH1 domain, respectively. The PCR products were cloned into pGEM-T Easy generating the pGEM-GD7 vector for the scIA^{d}/GD construct and the pGEM-OVA3 vector for the scIA^{d}/OVA construct. Following sequence verification, the scIA^{d}/GD and scIA^{d}/OVA gene fragments were isolated following digestion with NruI and EcORI. The purified fragments were subcloned into NruI/EcORI-cut pSUN7 resulting in the pJA-IgG2b/GD and pJA-IgG2b/OVA expression vectors for the scIA^{d}/GD-IgG heavy chain fusion and scIA^{d}/OVA-IgG heavy chain fusion, respectively.

TABLE 5

Oligonucleotides used in scIA^{d}/peptide-IgG
vector construction

| | |
|---|---|
| OPR225 | 5′-ATC GCC CCC TAC ACC AGC ACC CTG CTG CCC CCC GAG CTG AGC |
| | GAG ACC CCC G-3′ |
| OPR226 | 5′-CTA GCG GGG GCT TCG CTC AGC TCG GGG GGC AGC AGG GTG |
| | CTG GTG TAG GGG GCG AT-3′ |
| OPR237 | 5′-CAC CGC GTC GCG ACA GCT ACA GGT GTC CAC TCC ATC TCT CAG |
| | GCT GTT CAC-3′ |
| OPR238 | 5′-CAC CGC GTC GCG ACA GCT ACA GGT GTC CAC TCC GCC CCC TAC |
| | ACC AGC ACC CTG CTG CCC-3′ |
| OPR239 | 5′-CAC CGC GAA TTC GCT CCC TGA CAT GGG GGC TGG AAT CTC |
| | AGG TTC-3′ |

B. Mammalian Cell Production and Purification of Multimeric scIA^{d}/Peptide IgG Fusions.

To express multimeric scIA^{d}/GD-IgG molecules, mammalian cells were co-transfected with the pIAGMK and pJA-IgG2b/GD vectors and a vector, pPUR (Clontech) containing a gene that confers resistance to puromycin. Briefly, CHO cells were mixed with the linearized plasmid DNAs and transfected using Superfect transfection reagent by the manufacturer's instructions (Qiagen). The transfected cells were grown in non-selective IMDM medium overnight. Puromycin selective medium (IMDM medium, 20 μg/ml puromycin) was then added and the cells were transferred to 96 wells flat bottom tissue culture plates. Colonies of the cells co-transfected with the vectors became evident after 14-21 days. The transfectants carrying both the scIA^{d}/GD-Cκ and scIA^{d}/GD-Ig heavy chain vectors were expanded and were screened for expression of the soluble scIA^{d}/Ig fusion molecules by an ELISA specific for the IgG2b heavy chain/kappa chain multimer. Briefly, the ELISA involves coating 100 ng of goat anti-mouse IgG2b (Southern Biotechnology Associates) onto wells of Nunc Maxisorp plates in 100 μl of PBS (phosphate buffered saline) overnight at 4° C. The coating solution was removed and 200 1 10% FBS (fetal bovine serum)-PBS was added to each well for 2-3 hr at room temperature to block the wells. The blocking solution was removed and samples were added at 1001l/well and incubated 1 hr at room temperature. Plates were then washed 4 times with 200 μl wash buffer and 100 μl goat anti-mouse kappa chain-HRP (Southern Biotechnology Associates-Stock diluted 1/1000 in sample diluent) and incubated for 2 hr at room temperature. Plates were washed 6 times with wash buffer and 100 μl of TMB was added. After color development, 100 μl of 0.18M H₂SO₄ was added to quench the reaction and absorbance was read at 450 mm. Positive wells were also screened with an IA-specific ELISA described previously (WO 99/21572, U.S. Ser. No. 09/204,979). Based on the results of these assays, positive transfectants were selected and cloned by limiting dilution cloning as described previously (U.S. Ser. No. 09/204,979). The cloned transfectants were expanded and growth for 2-3 weeks in multiple T175 tissue culture flasks to produce 2-4 L of culture media containing the scIA^{d}/GD-IgG multimer. Similar work was carried out to generate CHO cell lines co-transfected with the pIADK and pJA-IgG2b/OVA vectors. These transfectants were expanded in T 175 flasks to produce 2-4 L of culture media containing the scIA^{d}/OVA-IgG multimer.

The sc-class II-IgG fusion molecules were purified by affinity chromatography using Protein A-Sepharose by standard methodologies (U.S. Ser. No. 09/204,979). In some cases, an additional chromatography step using an anti-bovine IgG Ab column was added to remove residual serum bovine immunoglobulin. SDS-PAGE analysis of purified scIA^{d}/OVA-Ig and scIA^{d}/GD-Ig molecules is shown in FIG. 8.

C. Activation of T Cells by Multi-Valent scIA^{d}/OVA-IgG Complexes.

The purified scIA^{d}/OVA-IgG and scIA^{d}/GD-IgG complexes were tested for their ability to activate the peptide-specific T cell hybridomas as measured by IL-2 production. This method involves coating the scIA^{d}/peptide-IgG protein and a rat anti-mouse IL-2 monoclonal antibody (PharMingen part no. 18161D) onto Maxisorp wells (NUNC Part No. 469949) in PBS overnight at 4° C. The coating solution was removed and 1×10⁵ T cells (DO11.10 or GD12) were added per well in 200 μl IMDM (MediaTech Cellgro, Part No. 15-016-CV) containing 10% FBS. Following an 8 hour incubation in a humid incubator at 37° C. with 10% CO₂, the plates were washed 3 times with wash buffer and an anti-IL-2 antibody conjugated to biotin (PharMingen part no. 18172D) was added at 100 ng/well in 100μl 10% FBS-PBS. Following an overnight incubation at 4° C., wells were washed 3 times with wash buffer and 250 ng of avidin peroxidase (Sigma part no. A3151) in 100 μl 10% FBS-PBS was added per well. Following incubation at 37° C. for 30 minutes, the wells were washed 5 times with wash buffer and 100 μl ABTS substrate (Kirkegaard and Perry, Part No. 5060-00) was added per well. Absorbance was measured at 405 nm. The result of an activation assay using DO11.10 cells is shown in Table 6 and using GD12 cells is shown in Table 7. Changing the antigenic peptide in the scIA^{d}-IgG construct from OVA to GD eliminated the ability of the single chain molecule to stimulate IL-2 production by the DO11.10 cells whereas the opposite specificity was seen for the GD 12 cells. The ability of the scIA^{d}/peptide-IgG protein to activate T cell responses was further characterized by comparison with the monovalent scIA^{d}/OVA molecules in the T cell activation assays. These assays showed that the immobilized scIA^{d}/peptide-IgG molecules were 2-8 fold more active in stimulating T cell responses than the scIA^{d}/peptide monomers.

TABLE 6
Activation of DO11.10 T cell by scIA^{d}/OVA complexes

| | IL-2 ELISA Values (A₄₀₅) | | | |
|---|---|---|---|---|
| ng complex/ | | | | scIA^{d}/ |
| well | scIA^{d}/OVA-IgG | scIA^{d}/GD-IgG | scIA^{d}/OVA | GD |
| 100 | 2.90 | 0.166 | 3.06 | 0.152 |
| 50 | 2.11 | 0.155 | 1.19 | |
| 25 | 0.758 | 0.166 | 0.209 | |
| 12.5 | 0.184 | 0.159 | 0.176 | |

TABLE 7
Activation of GD12 T cell by scIA^{d}/GD complexes

| | IL-2 ELISA Values (A₄₀₅) | | | |
|---|---|---|---|---|
| ng complex/ | | | scIA^{d}/ | |
| well | scIA^{d}/GD-IgG | scIA^{d}/OVA-IgG | GD | scIA^{d}/OVA |
| 100 | 3.16 | 0.115 | 0.484 | 0.123 |
| 50 | 1.76 | 0.122 | 0.138 | |
| 25 | 0.751 | 0.115 | 0.121 | |
| 12.5 | 0.347 | 0.123 | 0.134 | |

### EXAMPLE 4

### Generation of Multi-Valent Single-Chain DR2/MBP-IgG Complexes

A. Construction of Vectors for Expressing Multimeric Single-Chain DR2/MBP-IgG Complexes.

Construction of the vector expressing the scDR2/MBP-IgG heavy chain fusion was carried out as follows. The pDRHK vector served as template DNA for PCR amplifications using oligonucleotide primers OPR254 and OPR257 (see Table 8). The resulting PCR product carries the scDR2/MBP gene fusion and a 5′ BsiWI site and a 3′ EcORI site needed for cloning into the mammalian expression vector, pJRS355. This pcDNA3-based vector carries an expression cassette encoding immunoglobulin leader sequence and the human IgG1 C_{H}1-CH²-C_{H}3 domains. In this vector, the BsiPI and EcORI restriction sites are located near the 3′ end of the leader sequence and the beginning (5′ end) of the C_{H}1 domain, respectively. The PCR product was cloned into pGEM-T generating the JA-DR2-3 vector. Following sequence verification, the scDR2/MBP gene fragment was isolated from JA-DR2-3 following digestion with BsiWI and EcORI. The purified fragment was subcloned into BsiWI/EcORI-cut pJRS355 resulting in the pD2MIgH expression vector.

TABLE 8
Oligonucleotides used in scDR2/MBP-IgG vector construction

| | |
|---|---|
| OPR254 | 5′-CAC CGC GAA TTC GCT CCC TGG GAG AGG GCT TGG AGC ATC-3′ |
| OPR257 | 5′-CAC CGC GCG TAC GTC TTG TCC TAC GAC GAG AAC CCC GTG-3′ |

B. Mammalian Cell Production and Purification of Multimeric scDR2/MBP IgG Fusions

To generate cell lines expressing multimeric scDR2/MBP-IgG molecules, a CHO cell line previously transfected with the pDRKH expression vector was used. These cells are described in detail in U.S. Ser. No. 09/204,979. The pDRKH-transfected CHO cells were mixed with the linearized pD2MIgH and supercoiled pMACS plasmid DNAs and electroporated using a Gene Pulser (Biorad). The pMACS vector allows for the transient expression of a membrane bound CD4 protein. The co-transfected cells were selected for cell surface CD4 expression as described previously (U.S. Ser. No. 09/204,979). The cells were grown in non-selective IMDM medium in 96 wells flat bottom tissue culture plates. Colonies of the cells transfected with the vectors became evident after 14-21 days. The transfectants carrying both the scDR2/MBP-Cκ and scDR2/MBP-Ig heavy chain vectors were expanded and were screened for expression of the soluble scDR2-Ig fusion molecules by an ELISA specific for the heavy chain-HLA-DR fusion. Briefly, the ELISA involves coating 1 μg of sheep anti-Fd antiserum (The Binding Site) onto wells of Nunc Maxisorp plates in 100 μl of PBS (phosphate buffered saline) overnight at 4° C. The coating solution was removed and 300 μl 10% FBS (fetal bovine serum)-PBS was added to each well for 2-3 hr at room temperature to block the wells. The blocking solution was removed and samples were added at 100 μl/well and incubated 1 hr at room temperature. Plates were then washed 4 times with 200 μl wash buffer and 100 μl anti-HLA-DR monoclonal antibody L243-HRP (Stock diluted 1/1000 in sample diluent) and incubated for 2 hr at room temperature. Plates were washed 6 times with wash buffer and 100 μl of TMB was added. After color development, 100 μl of 0.18M H₂SO₄ was added to quench the reaction and absorbance was read at 450 nm. Positive transfectants were also screened with a DR-specific ELISA described previously (WO 99/21572, U.S. Ser. No. 09/204,979). Based on the results of these assays, positive transfectants were selected and cloned by limiting dilution cloning as described previously (U.S. Ser. No. 09/204,979). The cloned transfectants were expanded and growth for 2-3 weeks in multiple T 175 tissue culture flasks to produce 2-4 L of culture media containing the scDR2/MBP-IgG multimer.

### EXAMPLE 5

### Generation of Multi-Valent scIA^{d}/Peptide-IgM Molecules

A. Construction of Vectors for Expressing Soluble scIA^{d}/Peptide-IgM Fusion Molecules in Insect Cells.

As another approach of using the Ig scaffold to form multi-valent complexes, expression vectors were constructed encoding single-chain MHC/peptide molecules as fusion to the CH2-CH3-CH4 domains of IgM. The CH2-CH3-CH4 portion of the human IgM heavy chain was amplified using RT/PCR from the human B lymphoma cell line RL (ATCC number CRL-2261). Total RNA was prepared using the Qiagen total RNA kit (Part No. 14162) and cDNA was produced using the primer MBimr (5′-GGG GGG CCA TGG CTC GAG TCA GTA GCA GGT GCC AGC TG-3′). The cDNA served as template for PCR amplifications done using MBimr and MBimf (5′-GGGG GGA TCC GTG ATT GCT GAG CTG CCT CC-3′). The amplified gene fragment was ligated into the InVitrogen pGEM T-Easy vector (Part No. A1360) for sequence verification and was transferred as a BamHI/XhoI fragment into the PSL1190 vector (Pharmacia).

Gene fragments containing either the OVA 323-339 or HSV 246-261 antigenic peptides genetically linked to scIA^{d} fusion as described in pMBSC2.1 (U.S. Pat. No. 5,869,270) were amplified by PCR using primers MB101 (5′-GGG CCA TGG CTC TGC AGA TCC CCA GCC-3′) and MB100 (5′-GGG GGA TCC ACT AGC CCG GGA CCA GTG-3′). The PCR products were digested with NcoI and BamHI and transferred into the IgM-PSL1190 vector cut with NcoI/BamHI, resulting in pMB062 carrying the scIA^{d}/OVA-IgM construct and pMB063 carrying the scIA^{d}/GD-IgM construct. The entire gene fusion was transferred as a NcoI/HindIII fragment into the pBlubac III vector from InVitrogen (Part No. 120415), resulting in pMB060 carrying the 110 scIA^{d}/OVA-IgM construct and pMB061 carrying the scIA^{d}/GD-IgM construct.

B. Production of Soluble scIA^{d}/Peptide-IgM Fusion Molecules in Insect Cells.

The pMB060 and pMB061 vectors with insert were each used in a cotransfection and and recombinant virus was enriched from wild type AcMNPv by plaque purification (see D. R. O'Reilly et al. Baculovirus expression vectors: A laboratory manual. W. H. Freeman & Co. New York (1992) and the InVitrogen Bac-N-Blue Transfection Kit Instruction Manual Version G Catalog # K855-01). SF9 cells were used throughout.

Insect cells were infected at high multiplicity of infection (moi) in TNMFH insect cell media (JRH Biosciences part no. 51942-79P) with 10% fetal bovine serum (Hyclone SH 30071.03) to produce recombinant scIA^{d}/OVA-IgM protein. Approximately 96 hours post infection, the pH of the infection supernatant was adjusted to 8.0 with 10N NaOH, centrifuged at high speed (greater then 10,000×g) to remove particulate matter and 0.2 μm filtered. The processed supernatant was tested by ELISA to identify soluble fusion protein. The ELISA was carried out by coating 100 ng of M5/114 (ATCC TIB 120) anti -IA^{d} onto wells of a 96 well microtiter plate in 50 μl of PBS. A high protein rinse was done with 125 μl 10% FBS-PBS to remove unbound antibody. Samples are diluted into 10% FBS-PBS and were added at 100 1/well. After 1 hour at 37° C., the plates were washed 3 times with 375 μl of wash buffer (PBS with 0.05% Tween-20). Biotinylated goat anti-human IgM (Sigma part no. B-1265) was added at a 1:4000 dilution in 100 μl 10% FBS-PBS 10% FBS. After 1 hour at 37° C., the plates were washed 3 times with wash buffer. Avidin peroxidase (Sigma Part No. A3151) was added at 250 ng/well in 100 μl 10% FBS-PBS and incubated for 30 minutes at 37° C. Plates were then washed times with 375 μl of wash buffer and 100 μl of ABTS substrate (Kirkegaard and Perry, Part No. 5060-00) was added per well. Absorbance was measured at 405 nm.

Infection was done by adding 1 purified plaque to 50 ml of growth media containing SF9 cells at high viability. After 72 hours, 10 ml of the 50 ml infection was used as virus stock to infect 400 ml SF9 cells. After 96 hours, the ELISA was performed as above on supernatant from the 400 ml infection. Mean absorbance of duplicate wells is shown in Table 9 below. The specificity of the signal observed shown to be due to secretion of scIA^{d}/OVA-IgM into infection supernatant by SF9 cells.

TABLE 9
scIA^{d}/OVA-IgM ELISA of insect cell infection supernatants

| Sample Dilution | Absorbance (405 nm) |
|---|---|
| Undiluted | 1.274 |
| 1:2 | 0.964 |
| 1:4 | 0.739 |
| 1:8 | 0.488 |
| 1:16 | 0.361 |
| 1:32 | 0.279 |

Negative controls including sample diluent (A₄₀₅=0.171) and insect cell produced scIA^{d}/OVA without an IgM tail (A₄₀₅=0.175) showed background binding in this assay.

Supernatant from insect cells infected with the baculovirus carrying the scIA^{d}/GD-IgM expression cassette showed similar production of the scIA^{d}/GD-IgM molecules by this ELISA.

C. Purification of scIA^{d}/Peptide-IgM Fusion Molecules.

Filtered infection supernatant at pH 8.0 was applied onto a column of Protein A Sepharose cross-linked with M5, an anti-IA^{d} monoclonal antibody. Following sample application, the immunoaffinity column was washed with 20 mM Tris-HCl, pH 8.0 until A₂₈₀ nm baseline was reached. The antibody column was then washed with the same buffer containing 1M NaCl as above to remove non-specific bound proteins. The scIA^{d}/OVA-IgM protein was then eluted with 50 mM glycine NaOH, pH 11. The eluted peak protein (monitored by A₂₈₀ₙₘ) was immediately adjusted to pH 8.0 with 2M glycine, pH 2.0, concentrated and buffer exchanged into PBS by ultrafiltration. The purified sample was stored at 4-8° C. The purity and functionality of the scIA^{d}/OVA-IgM sample was tested by SDS-PAGE and T cell activation, respectively. Purified scIA^{d}/OVA-IgM from this preparation showed no contaminating bands on a Coomassie stained polyacrylamide gel and total protein was 60 μg/ml by total protein assay. When analyzed without reduction of the interchain disulfide bonds, the scIA^{d}/OVA-IgM protein appear to be composed of higher order (trimers and greater) multimers. Similar purification methods were used to generate scIA^{d}/GD-IgM protein from infected insect cell culture supernatant.

D. Activity of scIA^{d}/Peptide-IgM Complexes in Stimulating T Cell Responses.

The purified scIA^{d}/OVA-IgM complexes were tested whether they could activate the OVA-specific DO11.10 T cell hybridoma as measured by IL-2 production. This method involves coating the scIA d/OVA-IgM protein (or scIA^{d}/GD-IgM protein as a control) and a rat anti-mouse IL-2 monoclonal antibody (PharMingen part no. 18161D) onto Maxisorp wells (NUNC Part No. 469949) in PBS overnight at 4° C. The coating solution was removed and 1×10⁵ DO11.10 cells were added per well in 200 μl IMDM (MediaTech Cellgro, Part No. 15-016-CV) containing 10% FBS. Following an 8 hour incubation in a humid incubator at 37° C. with 10% CO₂, the plates were washed 3 times with wash buffer and an anti-IL-2 antibody conjugated to biotin (PharMingen part no. 18172D) was added at 100 ng/well in 100 μl 10% FBS-PBS. Following an overnight incubation at 4° C., wells were washed 3 times with wash buffer and 250 ng of avidin peroxidase (Sigma part no. A3151) was added per well in 100 μl 10% FBS-PBS. Following incubation at 37° C. for 30 minutes, the wells were washed 5 times with wash buffer and 100 μl BTS substrate (Kirkegaard and Perry, Part No. 5060-00) was added per well. Absorbance was measured at 405 nm. The results of one activation assay are shown in Table 10 below changing only the antigenic peptide in the scIA^{d}-IgM construct from OVA to GD eliminate ability of the single chain molecule to stimulate IL-2 production by the DO11.10 cells. Neither DO11.10 cells with immobilized anti-IL2 antibody alone nor DO11.10 cells with scIA^{d}/GD-IgM and anti-IL2 antibody resulted in IL-2 secretion.

TABLE 10
Activation of DO11.10 T cell by
multi-valent scIA^{d}/OVA-IgM complexes

IL-2 ELISA Values (A₄₀₅)

| ng complex/ | Stimulation by | Stimulation by |
|---|---|---|
| well | scIA^{d}/OVA-IgM | scIA^{d}/GD-IgM |
| 300 | 0.853 | 0.248 |
| 100 | 1.700 | 0.221 |
| 33 | 1.637 | 0.206 |
| 11 | 0.601 | 0.188 |
| 4 | 0.230 | 0.179 |
| 1 | 0.195 | 0.184 |
| 0.3 | 0.192 | 0.189 |
| Anti-IL-2 alone | 0.214 | 0.200 |

To further characterize the ability of the scIA^{d}/peptide-IgM protein to activate T cell responses, the multi-valent scIA^{d}/peptide-IgM molecules were directly compared to the monovalent scIA^{d}/peptide molecules in the T cell activation assays described above. The results of assays using DO11.10 and GD 12 T cells are shown in Table 11 and 12, respectively. These assays showed that the immobilized scIA^{d}/peptide-IgM molecules were 5-10 fold more active in stimulating T cell responses than the scIA^{d}/peptide monomers. The peptide specificity of the response was observed for both monomeric and multimeric scIA^{d}/peptide molecules.

TABLE 11
Activation of DO11.10 T cell by scIA^{d}/OVA complexes

| | IL-2 ELISA Values (A₄₀₅) | | | |
|---|---|---|---|---|
| ng complex/ | | | | scIA^{d}/ |
| well | scIA^{d}/OVA-IgM | scIA^{d}/GD-IgM | scIA^{d}/OVA | GD |
| 250 | 1.475 | 0.193 | 0.665 | 0.180 |
| 125 | 1.610 | 0.180 | 1.059 | 0.179 |
| 62 | 1.334 | 0.157 | 1.032 | 0.164 |
| 31 | 1.417 | 0.165 | 0.646 | 0.213 |
| 15 | 1.178 | 0.140 | 0.397 | 0.152 |
| 7 | 0.838 | 0.148 | 0.236 | 0.163 |
| 3 | 0.529 | 0.140 | | |
| 1 | 0.268 | 0.153 | | |

TABLE 12
Activation of GD12 T cell by scIA^{d}/GD complexes

| | IL-2 ELISA Values (A₄₀₅) | | | |
|---|---|---|---|---|
| ng complex/ | | | | scIA^{d}/ |
| well | scIA^{d}/OVA-IgM | scIA^{d}/GD-IgM | scIA^{d}/OVA | GD |
| 250 | 0.232 | 1.406 | 0.230 | 0.688 |
| 125 | 0.235 | 1.324 | 0.189 | 0.913 |
| 62 | 0.201 | 1.076 | 0.183 | 0.863 |
| 31 | 0.190 | 0.982 | 0.181 | 0.450 |
| 15 | 0.185 | 0.833 | 0.160 | 0.174 |
| 7 | 0.170 | 0.799 | 0.178 | 0.156 |
| 3 | 0.188 | 0.623 | | |
| 1 | 0.156 | 0.404 | | |

The results from Examples 2, 3 and 5 clearly show that the multi-valent forms of the scIA^{d}/peptide complex are more potent at stimulating T cell responses than the monovalent form. These complexes would be preferred in screening methods where T cell responses to the MHC/peptide complex require a elevated threshold of TCR:MHC/peptide interaction. Such screening methods using T cells expressing recombinant scTCR-CD3ζ fusions are described in Examples 11-14 below. The multi-valent MHC/peptide complexes are also preferred in screening methods that rely on the detection of interactions between the MHC/peptide complex and membrane-bound TCR (see Example 15) or purified TCR (see Examples 16 and 17).

### EXAMPLE 6

### Generation of Multi-Valent DO11.10 Single-Chain T Cell Receptor Complexes.

A. Construction of Vectors Expressing the DO11.10 scTCR/Murine IgG2b Fusion Molecule.

As described above, development of screening methods to detect interactions between the MHC antigen molecule and the TCR can be greatly facilitated by the availability of monovalent and preferably multi-valent MHC antigen complexes and TCR reagents. Methods for generating monovalent and multi-valent single-chain TCR (scTCR) molecules have been described previously (Weidanz, et al. 1998. J. Immunol. Methods 221:59, U.S. patent application Ser. Nos. 08/813,731 and 08/943,086). This example describes use of these methods to create multi-valent DO11.10 scTCR molecules for screening application such as those described in Examples 16 and 17.

The cloning of the DO11.10 scTCR has been described in the pending U.S. application Ser. No. 08/813,731 (“Fusion Proteins Comprising Bacteriophage Coat Protein and A Single-chain T Cell Receptor”) and Ser. No. 08/943,086 (“Fusion Proteins Comprising Single-Chain T-Cell Receptor and an Immunoglobulin Light Chain Constant Region”).

The mammalian IgG2b expression vector pSUN7 has been described in the most recent TCR patent application (“Polyspecific Binding Molecules and Uses Thereof”, Ref. No. 48,531-P).

For the construction of the DO11.10 scTCR/murine IgG2b fusion molecule, the pSUN7 expression vector was digested with NruI and EcORI (which drops out the scFv originally cloned into the vector). The DO111.10 scTCR was reamplified from pSUN23 using specific primers KC267 (5′ NruI) and KC268 (3′ EcORI) (see Table 13). The PCR products were cloned into the pGEM-T Easy vector for sequencing. The correct DO11.10 scTCR fragment was isolated by restriction digest/gel purification and then ligated into the NruI/EcORI-digested pSUN7 expression vector to create the final DO11.10 scTCR/murine IgG2b expression vector.

TABLE 13
Oligonucleotides used in cloning

| | |
|---|---|
| KC267 | 5′-GAG GTG TCG CGA GAG CAG GTG GAG CAG CTT CC-3′ |
| KC268 | 5′-GTG GAG GAA TTC GTC TGC TCG GCC CCA G-3′ |
| KC303 | 5′-GAG GTG GTT AAC GAT CCC AAA CTC TGC TAC-3′ |
| KC304 | 5′-GAG GTG ATC GAT AAG TGT ACT TAC GTT TTT AGC GAG GGG |
| | GCA GGG C-3′ |
| KC312 | 5′-GAG GTG GTT AAC GAT CCC AAA CTC TGC TAC TTG CTA GAT |
| | GGA ATC CTC-3′ |

B. Mammalian Expression of the DO11.10 scTCR/Murine IgG2b Fusion Molecules.

The CHO cells were prepared for transfection by washing with cold DPBS. Approximately 1 cells were resuspended in DPBS and mixed with 20 μg of PvuI-linearized DO11.10 scTCR/murine IgG2b DNA. After 5 minutes on ice, the cells were electroporated using a Gene Pulser (BiORad) set to deliver one pulse of 250 volts, 0.25 μFd. The pulsed cells were placed on ice for 5 minutes. The cells were diluted into 10 ml of IMDM (with 10% FBS) and grown in T-25 cm² flasks overnight at 37° C. with 5% CO₂.

The transfected cells were diluted 1:200 and plated out the next day in 96 well plates in selective medium (i.e. 10% FBS-IMDM with 0.75 mg/ml G-418 sulfate).

Approximately two weeks later, colonies were tested by ELISA for protein expression. A 96 well plate (Nunc Maxisorp) was coated with the KJ-1 mAb (this mAb recognizes correctly folded DO11.10 TCR) in PBS overnight at 4° C. After blocking with 10% FBS-PBS for 1 hour, the colony supernatants were added and incubated for 30 minutes at room temperature. After washing, an anti-murine IgG2b-HRP conjugated polyclonal antibody (Caltag) was added for 30 minutes. The ELISA was developed using TMB substrate and quenched with 0.1N sulfuric acid. The absorbance was read at 450 nm.

Only 10% of the tested colonies were positive in the ELISA. Three candidates were chosen for expansion and primary cloning by limiting dilution.

Approximately ten days later, the primary clones were tested by ELISA to select for the highest protein producing cell line. The ELISA was similar to the assay described above except the F23.1 mAb (this mAb detects a portion of the TCR V beta 8 chain) was used in place of the KJ-1 mAb. Four candidates were expanded and the clone with the highest TCR expression level (#13-1) was chosen for further characterization studies.

C. Generation of Multi-Valent DO11.10 scTCR Fusion Molecules.

Expression of the DO10.10 scTCR/murine IgG2b fusion molecule in mammalian cells produces monomers and dimers of TCR as shown in FIG. 9. The addition of a kappa chain fusion to complement the IgG2b heavy chain fusion could lead to the formation of tetrameric molecules. To test this hypothesis, it was first necessary to establish that a scTCR/IgG2b fusion molecule could pair effectively with a scTCR/kappa fusion molecule.

The cloning of the DO11.10 scTCR/murine kappa fusion molecule has been described in the pending U.S. application Ser. No. 08/943,086. To demonstrate heavy chain/kappa chain pairing, a transient transfection experiment was set up following Qiagen's Superfect Transfection Reagent protocol for transient transfection of adherent cells. Briefly, 2.5×10⁵ Cos-7 cells were seeded (per well) in a 6 well plate. The next day, 2 μg of DNA (1 μg of DO11.10 scTCR/kappa plus 1 μg of DO11.10 scTCR/IgG2b) was added to 100 μl of IMDM medium. Ten 1 of the Superfect Reagent (Qiagen) was then added and mixed. After a 10 minutes incubation at room temperature to allow DNA/dendrimer formation, 600 μl of IMDM containing 10% FBS was added to the complexes. The COS cells were washed with DPBS and the complexes were pipetted onto the cells. After 2 hours at 37° C., the cells were washed again and fresh IMDM containing 10% FBS was added to the wells. The transfections were incubated at 37° C. with 5% CO₂ for 3 days before being assayed.

Supernatant from the transient transfections was tested by ELISA for pairing of the scTCR/IgG2b fusion molecule with the scTCR/kappa fusion molecule. A 96 well plate (Nunc Maxisorp) was coated with goat anti-murine kappa polyclonal antibody (Southern Biotech) in PBS overnight at 4° C. After blocking with 10% FBS-PBS for 1 hour, the transient supernatants were added and incubated for 30 minutes at room temperature. After washing, an anti-murine IgG2b-HRP conjugated polyclonal antibody (Caltag) was added for 30 minutes. The ELISA was developed using TMB substrate and quenched with 0.1N sulfuric acid. Absorbance (read at 450 run) was 40 times greater than the negative control, clearly indicating that the successful pairing of scTCR/kappa with scTCR/IgG2b.

The following examples describe the generation of cell lines expressing recombinant T cell receptors.

### EXAMPLE 7

### Generation of a Murine T Cell Hybridoma Carrying a Recombinant T Cell Receptor.

Development of cell-based screening methods to detect interactions between the MHC antigen molecule and the TCR can be greatly facilitated by the availability of immortalized T cell lines expressing the appropriate TCR. Fusions between isolated T cells and thymoma cells (i.e. BW5147) have been made to generate murine T cell hybridomas. However the same methods can not be readily applied to the generation of immortalized human T cell lines. As more generalized method, immortalized T cell lines have been generated by transfection to express recombinant TCRs on the cell surface (see Engel et al. 1992. Science 256:1318; Hastings et al. 1996. J. Immunol. 157:3460; Brawley, J. V. and P. Concannon. 1999. J. Immunol. 163:4946). A similar approach is described in this and the next two examples. In each case, a fusion was made between the extracellular antigen recognition domains of the TCR and the intracellular signaling domain of the CD3-ζ chain. When transfected into T cells, the fusion molecules showed functional activity when stimulated with MHC-peptide complex. In this example, the T cell line used in the transfection was a hybridoma expressing an endogenous TCR on its surface. In examples 8 and 9, the T cell lines used do not express functional endogenous TCRs on their surfaces.

A. Construction of the DO11.10 scTCR/CD3ζ Fusion Molecules.

The cloning of the DO11.10 scTCR has been described in the pending U.S. applications Ser. No. 08/813,731 (“Fusion Proteins Comprising Bacteriophage Coat Protein and A Single-chain T Cell Receptor”) and Ser. No. 08/943,086 (“Fusion Proteins Comprising Single-Chain T-Cell Receptor and an Immunoglobulin Light Chain Constant Region”).

In the most recent patent application (“Polyspecific Binding Molecules and Uses Thereof”, Ref. No. 48,531-P), the construction of a pGEM T-Easy-based “shuttle vector” was described. The vector consisted of a scTCR amplified as a 5′ AgeI-3′ HpaI/BspEI/NruI/ClaI DNA fragment cloned into the pGEM-T Easy vector. The 3′ end then served as a polylinker region for the addition of a linker, a scFv and a purification tag to create a bispecific molecule.

For the construction of the DO11.10 scTCR/CD3ζ fusion molecules, the DO11.10 scTCR/linker/scFv/tag/pGEM-T Easy “shuttle vector” was digested with HpaI and ClaI to remove the polylinker region, leaving only the DO11.10 scTCR as a 5′ AgeI-3′ HpaI fragment in pGEM-T Easy.

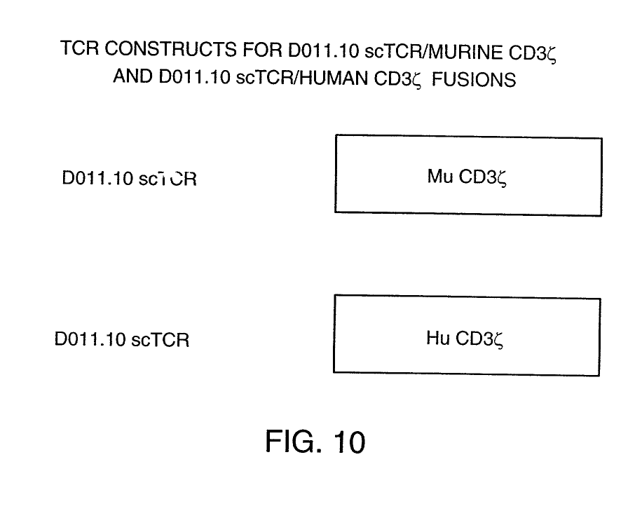

The CD3ζ chains were amplified from cDNA (murine cDNA was made from 2B4 T cell hybridoma RNA; human CD34 cDNA was a kind gift from Dr. Linda Sherman's laboratory) using mouse specific primers KC312 (5′ HpaI) and KC304 (3′ ClaI) or human specific primers KC303 (5′ HpaI) and KC304. The KC304 primer also encoded for a 5′ stop codon and splice site. The PCR products were cloned into the pGEM vector (the original vector from Promega) for sequencing. Correct CD3ζ fragments were isolated by restriction digest/gel purification and then ligated into the HpaI/ClaI-digested DO11.10/pGEM vector (described above) to create either a DO11.10 scTCR/murine CD3ζ or DO11.10 scTCR/human CD3ζ fusion construct in pGEM-T Easy, shown in FIG. 10.

The mammalian IgG kappa vector pSUN9 (described in the pending U.S. Application No. 08/943,086) was selected for expression of the scTCR/CD3′ fusion molecules. The expression vector and the fusion constructs (in pGEM-T Easy) were both digested with AgeI and ClaI. The scTCR/CD3ζ DNA fragments were isolated and ligated into the cut pSUN9 vector to create the final mammalian cell expression vectors, DO11.10 scTCR/murine CD34 and DO11.10 scTCR/human CD3ζ.

B. Generation of 2B4 Cells Expressing Membrane-Bound DO11.10 scTCR/Murine CD3ζ Fusion Molecules.

The 2B4 T cell hybridoma is the result of a fusion of T cells from B 10.A mice with the BW5147 cell line. 2B4 cells recognize pigeon cytochrome C(PCC) when presented in the context of IE^{k}.

2B4 cells were prepared for transfection by washing with cold DPBS. Approximately 10⁷ cells were resuspended in DPBS and mixed with 20 μg of PvuI-linearized DO11.10 scTCR/murine CD34 DNA. After 5 minutes on ice, the cells were electroporated using a Gene Pulser (BiORad) set to deliver one pulse of 250 volts, 960 μFd. The pulsed cells were placed on ice for 5 minutes. The cells were diluted into 10 ml of 10% IMDM and grown in a T-25 cm² flask overnight at 37° C. with 5% CO₂.

After 24 hours, the cells were transferred to a T-75 cm² flask and 40 ml of selective medium (i.e. 10% FBS-IMDM with 1 mg/ml G418) was added. Three days later, the “bulk” 2B4 transfectants were plated out in 96 well plates at 10, 100 or 1000 cells per well in selective medium. Approximately two weeks later, the plated transfectants were screened for DO11.10 expression using flow cytometry. The cells were stained with biotinylated-F23.1 mAb for 30 minutes on ice. The F23.1 mAb detects a portion of the TCR V beta 8 chain; therefore it binds to the DO11.10 TCR but not the 2B4 TCR. The cells were gently spun, the supernatant was aspirated and streptavidin-labeled CyChrome was added. After 15 minutes on ice, the cells were washed twice with 1% FBS/DPBS and resuspended. Cell staining results were acquired on a Becton Dickinson FACScan instrument.

Approximately 90% of the tested colonies were positive for DO11.10 scTCR expression. Two candidates were selected for primary cloning by limiting dilution.

Two weeks later, primary clones were screened by flow cytometry as previously described. Six clones, with various levels of CD4 and DO11.10 scTCR expression, were chosen for further characterization studies. Use of these clones in T cell activation studies using recombinant scIA^{d}/OVA complexes is described in Example 11.

### EXAMPLE 8

### Generation of murine T cell lines carrying recombinant T cell receptor and recombinant CD4 molecules.

A. Generation of BW5147 cells expressing membrane-bound DO11.10 scTCR/CD3ζ fusion molecules.

The BW5147 (BW) cell line is a mouse thymoma that lacks surface expression of TCR alpha and beta chains. (This cell line is often used as the fusion partner with T cells to generate T cell hybridomas.)

The BW cells were prepared for transfection by washing with cold DPBS. Approximately 10⁷ cells were resuspended in DPBS and mixed with 20 μg of PvuI-linearized DO11.10 scTCR/murine CD3ζ DNA. In a separate tube, 10 cells were mixed with 20 g of PvuI-linearized DO11.10 scTCR/human CD3ζ DNA. After 5 minutes on ice, the cells were electroporated using a Gene Pulser (BiORad) set to deliver one pulse of 250 volts, 960 μFd. The pulsed cells were placed on ice for 5 minutes. The cells were diluted into 10 ml of 10% IMDM and grown in T-25 cm² flasks overnight at 37° C. with 5% CO₂.

After 24 hours, the cells were transferred to T-75 cm² flasks and 40 ml of selective medium (i.e. 10% FBS-IMDM with 1 mg/ml G418) was added to each. Three days later, the “bulk” BW transfectants were plated out in 96 well plates at 10, 100 or 1000 cells per well in selective medium. A “bulk” transfected culture was also maintained for each.

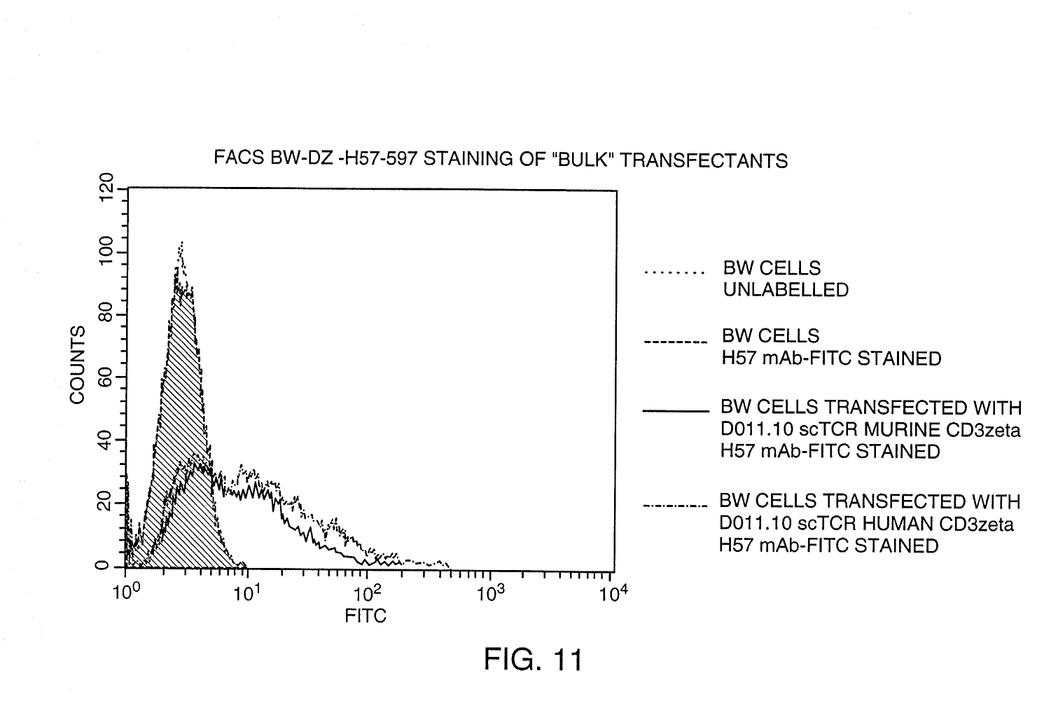

Approximately two weeks later, the “bulk” transfected cultures were screened for positive transfectants using flow cytometry. The cells were stained with FITC-conjugated H57-597 mAb for 30 minutes on ice. The H57-597 mAb detects a linear epitope of the TCR Cβ chain. After washing twice with 1% FBS/DPBS, the cells were resuspended in the same buffer. Cell staining results were acquired on a Becton Dickinson FACScan instrument. The “bulk” transfected cultures both showed a broad range of TCR expression, as shown in FIG. 11.

The “bulk” transfected cultures were also tested in an activation assay using immobilized H57-597 mAb. A 96 well plate was coated with 300 ng/well of mAb overnight. The following day, 10⁵ cells were added to each well and the assay was incubated overnight at 37° C. with 5% CO₂. The supernatants from the assay were tested in a murine IL-2 sandwich ELISA as described previously (WO 99/21572, U.S. Ser. No. 09/204,979). The “bulk” transfected cultures were specifically stimulated to produce IL-2 based on this assay.

The transfectants that grew on the 1000 cells/well plate were screened by flow cytometry for expression of the DO11.10 scTCR/murine CD3ζ molecule as described in Example 7. One-third to two-thirds of the tested candidates were positive for TCR expression. The DO11.10 scTCR/murine CD3ζ transfectant (BW/D-Z #8) with the highest expression level was primary cloned, expanded and further characterized.

Primary clones from BW/D-Z #8 were screened by flow cytometry. One clone (#8-39) was selected as the best cell line to use for additional characterization studies.

The BW/D-Z cell line was to compare to the original T cell hybridoma DO11.10 from which the scTCR gene was cloned. When the two cell lines were stained side-by-side, it was evident that the level of TCR expression on the transfected BW cells was higher than on the native T cell hybridoma as disclosed by the H57-597 mAb. Both cell lines were stimulated to produce IL-2 in the presence of APCs (A20 cells) loaded with the OVA peptide. However, when purified single-chain IA^{d}/OVA was immobilized on a plate, the DO11.10 T cell hybridoma was activated but the BW/D-Z cell line failed to produce detectable levels of IL-2. It was believed that an accessory molecule, involved in the stabilization of the MHC antigen/TCR interaction, might be the missing component in the activation assay. The CD4 molecule was the most logical choice as it is directly involved in TCR binding to MHC molecule and the BW/D-Z cells do not express CD4 on their surface.

B. Construction of Vectors for Expression of Membrane Bound CD4 Receptors.

The CD4 receptor used for this construct was originally isolated by PCR amplification of cDNA generated from the mouse T cell hybridoma GD12 (Grammer, S. F. et al. J. Immunol. 145:249 (1990)).

Two different CD4 receptor genes were constructed: a full-length gene containing the extracellular, transmembrane (TM) and cytoplasmic (CM) domains, and a truncated receptor lacking a cytoplasmic domain. The latter construct was conceived to elucidate the contribution of CD4 extracellular receptor domain to the TCR:MHC/peptide interaction and to the amplification of this interaction through its cytoplasmic domain.

The two CD4 receptor genes were generated by PCR and were cloned into the expression vector pcDNA3.1: Hygro+.

To generate the full length CD4 gene, two oligonucleotide primers (see Table 14) were designed to amplify a 1550 bp PCR fragment containing the entire open reading frame of the CD4 receptor gene and 169 bp of upstream sequence. Oligonucleotides CDF1 and CDF3 were used in PCR to amplify the full-length CD4 gene while CDF 1 and CDF4 were used to amplify the CD4 deltaCM gene. Both DNA fragments were constructed with flanking HindIII and XhoI restriction sites. Since an XhoI site is present 95 bp upstream of the initiating methionine of CD4, to clone the full-length gene, a 1480 bp XhoI fragment was generated and ligated to an XhoI digested pcDNA3.1 Hygro+vector (Clontech). The resulting plasmid was linearized with BglII and used to transfect BW/D-Z cells (as described in Example 7).

A similar strategy was devised to generate the truncated CD4 gene. The oligonucleotide primers CDF1 and CDF4 were used to amplify a 1497 bp PCR fragment containing the open reading frame of the CD4 receptor gene lacking the cytoplasmic domain and containing 169 bp of upstream sequence. This fragment was flanked with HindIII and XhoI restriction sites. An XhoI site is present 95 bp upstream of the initiating methionine. A 1427 bp XhoI fragment was ligated to a XhoI digested pcDNA3.1 Hygro+vector (Clontech) and the resulting plasmid was linearized with BglII and used to transfect BW/D-Z cells.

TABLE 14

Oligonucleotide primers used in cloning of murine CD4
and CD4 delta CM

| | |
|---|---|
| Designation | Sequence |

| | |
|---|---|
| CDF1 | 5′-GGG GAA GCT TTT TTC ATT TAC GAA CAT CTG TGA AGG C-3′ |
| CDF3 | 5′-GGG GTC GAG TTA TCA GAT GAG ATT ATG GCT CTT CTG C-3′ |
| CDF4 | 5′-GGG GCT CGA GTT ATC AGC GCT GTT GGT GCC GGC ACC TGA |
| | CAC AGC AGA GGA TGC AGA G-3′ |

C. Generation of BW:D-Z Cells Expressing Membrane-Bound CD4 Receptors.

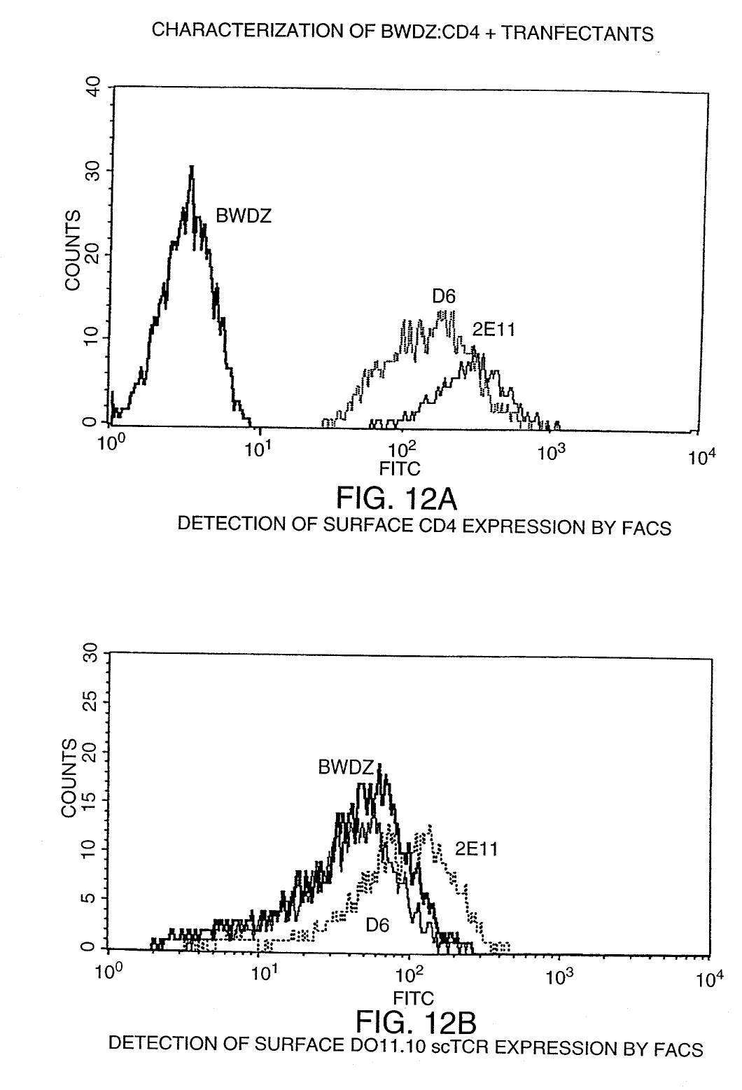

BW/D-Z cells transfected with either the full length or the truncated CD4 genes were grown in IMDM medium containing 10% fetal bovine serum (FBS) and 1 mg/ml Hygromycin. Resistant colonies were cloned by limited dilutions and were examined by FACS analysis for surface expression of CD4 receptor. Approximately 1 cells were mixed with FITC labeled rat anti murine CD4 antibody (PharMingen) in 50 μl of PBS with 1% serum. Cell surface expression of the recombinant CD4 receptor was analyzed by flow cytometry as shown in FIG. 12A.

Positive clones were also examined by FACS analysis for membrane bound expression of DO11.10 scTCR. Approximately 10⁵ cells were mixed with anti-TCR beta-chain biotinylated antibody H57. The cells were washed and mixed with streptavidin-FITC and analyzed by flow cytometry as shown in FIG. 12B. Of the double positive cell lines examined, BWDZ:CD4+(2E11) and BWDZ:deltaCM (D6) were selected for further TCR:MHC/peptide interaction studies using cognate OVA-peptide presenting cells (APCs). The same T cell lines were used in stimulation studies with immobilized recombinant scIA^{d}/OVA molecules described in Example 12.

D. Stimulation of BWDZ:CD4+ T Cells by Peptide-Loaded APCs.

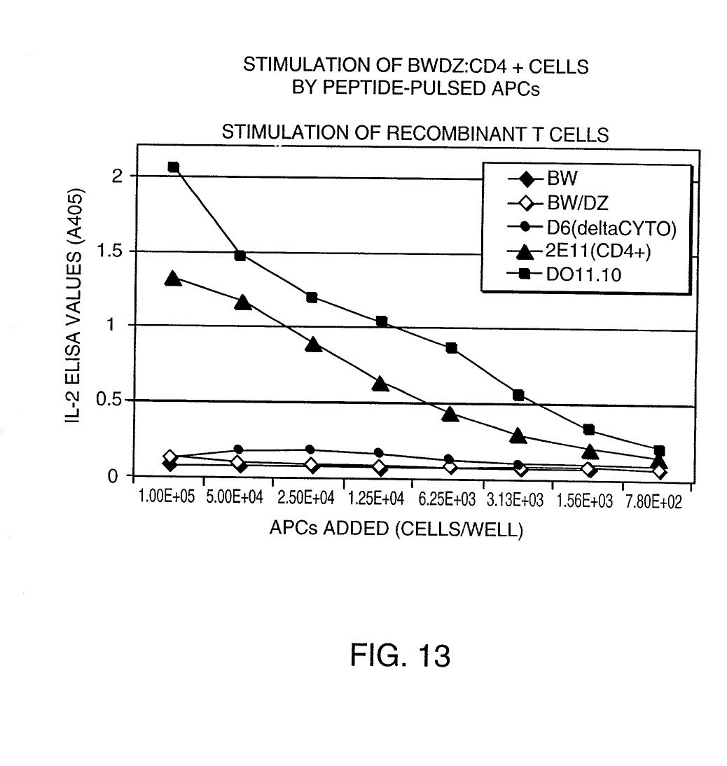

To demonstrate that BWDZ:CD4+cells are stimulated with cognate APCs, A20 cells were mixed with OVA peptide. A two-fold serial dilution of APCs was made and mixed with 105 T cells. The cell mix was incubated in 200 μl of IMDM medium containing 10% FBS at 37° C. overnight. Production of IL-2 in the culture media was determined by an IL-2 specific ELISA as described previously. Production of IL-2 indicates the extent of T cell stimulation, as shown in FIG. 13. DO11.10 cells were used as a positive control and BW cells were used as a negative control. The IL-2 production of the two cell lines expressing CD4 was compared to that of the 2B4/DZ. The addition of the full-length CD4 accessory molecule restored IL-2 production of the BW/DZ cells up to 75%-80% of that of DO11.10 cells. The truncated CD4 molecule lacking the cytoplasmic domain restored only 10% by comparison. As predicted, CD4 cytoplasmic domain plays a major role in amplifying the TCR:MHC/peptide interaction, through its recruitment of lck⁵⁶.

### EXAMPLE 9

### Generation of a human T cell line expressing recombinant human T cell receptor.

A. Construction of Vectors Containing cDNA Clones of the TCR α and β Chains From the Human MBP-Reactive Cell Line E11.

The human T cell line E11 recognizes and reacts to a peptide from myelin basic protein (MBP 83-102, YDENPVVHFFKNIVTPRTPP) in the context of the human MHC II protein HLA-DR2 (DRB1*1501). It was originally isolated from a patient with multiple sclerosis and was provided to us as a frozen cell pellet by Corixa Corp.

Total RNA was prepared from 1×10⁸ cells using the RNeasy kit from Qiagen. After dilution in RLT (lysis) buffer, the preparation was divided into aliquots equivalent to 1×10⁷ cells for further processing. Final RNA concentration was determined spectrophotometrically. First strand cDNA was prepared using the 5′ RACE cDNA synthesis primer and the SMART II oligo from Clonetech (Menlo Park, Calif.) as outlined in their user manual (PT3269-1, March 1999). Oligonucleotides used are listed in Table 15. PCR amplification of the first strand (RACE) was performed according to instructions with the following exceptions; the reaction volume was doubled to 100 μl and ExTaq enzyme and buffer (Panvera Corp.) were used. The gene specific primers used in the reaction primed in the constant region of the α and β chain, either close to the variable region (“N terminal”) or at or near the end (“C terminal”). The RACE reactions yielded products of the expected size for all four of the combinations and these products were then cloned into pGEM-T Easy (Promega Corp., Madison, Wis.) for sequence analysis. Analysis of 12 β chain candidates yielded two variable region gene sequences; 11 of 12 clones were TRVB12-4 and one of 12 clones was TRVB14 (IGMT nomenclature, see ImMunoGeneTics databases, http://imgt.cnusc.fr:8104). Analysis of 14 α chains yielded two sequences in approximately equal frequency; 5 of 14 clones were TRAV9s1, 7 of 14 clones were TRAV22s1 and the remaining two were TRAVls3 and TRAV4s1 (IGMT nomenclature).

TABLE 15

Oligonucleotide primers used in cloning of TCR
α and β chains from the E11 cell line

| | |
|---|---|
| designation | sequence |

| | |
|---|---|
| SMART II | 5′-AAG CAG TGG TAA CAA CGC AGA GTA CGC GGG-3′ |
| 1^{st} strand cDNA (Clonetech) | |
| 5′-RACE | 5′(T)₂₅N₋₁N-3′ (N = A, C, G, or T; N − 1 = A, G, or T) |
| 1^{st} strand cDNA (Clonetech) | |
| Universal | long (0.2 μM) |
| primer mix | 5′-CTA ATA CGA CTC ACT ATG GGC AAG CAG TGG TAA CAA |
| | CGC AGA GT-3′ |
| | short (1.0 μM) |
| | 5′-CTA ATA CGA CTC ACT ATG GGC-3′ |
| RACE 5′ primer (Clonetech) | |
| VW510 | 5′-ATC CTT TCT CTT GAG CAT GGC CAT C-3′ |
| RACE 3′, β chain, C terminal | |
| VW511 | 5′-CAC AGC GAC CTC GGG TGG GAA CAC-3′ |
| RACE 3′, β chain, N terminal | |
| VW512 | 5′-GCT GGA CCA CAG CCG CAG CGT CAT G-3′ |
| RACE 3′, α chain, C terminal | |
| VW513 | 5′-CAG CTG GTA CAC GGC AGG GTC AGG-3′ |
| RACE 3′, α chain, N terminal | |

B. Construction of Mammalian Expression Vectors Containing cDNA Clones of the TCR α and β Chains from the Human MBP-Reactive Cell Line E II as Fusions to Human CD3ζ and Transfection of Mammalian Cells.

Chimeric molecules were constructed that contained either the TCR α or β chain (s) from the E11 cell line fused genetically to the transmembrane and cytoplasmic domain of human CD3ζ. Engel, Ottenhoff and Klausner (Science 256:1318-1321, 1992) demonstrated that murine constructs, similar to those described below for human, when expressed in a rat basophilic leukemia cell line produced cells which could be activated by the specific MHC antigen molecules.

The E11 clones containing variable regions for the β chain TRVB 12-4 and the α chains TRVA22s1 and 9s1 and their corresponding constant regions served as templates for amplification with primers to introduce restriction endonuclease sites 5′ of the initiating methionine and at the end of the constant domain before the transmembrane region. The a chain clones used for amplification were pTVW443-1 and-7 (α 9s1 and 22s1 respectively) and oligonucleotide primers used to amplify them are listed in Table 16. Briefly, the 5′ primers contained the restriction sites SalI and KpnI. The 3′ primer contains a HpaI restriction site that allows an inframe fusion with a human CD3ζ, fragment upon digestion and ligation (i.e. in the DO11.10 scTCR/human CD34 pGEM-T Easy vector described in Example 7). The β chain clone used for amplification was pTVW441-2 (β 12-4). The 5′ primer for this construct contained the restriction sites SalI and HindIII and the 3′ primer contained the HpaI site for inframe fusion to human CD3ζ. Amplification by PCR using the oligonucleotides described generated appropriate fragments that were then cloned into pGEM-T Easy to make plasmids pTVW450-1, pTVW451-1 and pTVW449-1 (containing fragments encoding α9s1, α22s1 and β12-4 respectively). These plasmids were digested with SalI and HpaI to yield fragments necessary for cloning into the DO11.10 scTCR/human CD3ζ pGEM-T Easy vector cut with the same enzymes. The resulting plasmids pTVW455-1, pTVW454-1 and pTVW453-1 (encoding α9s1, α22s1 and β12-4 respectively) contained the extracellular domains of TCR α or β chains fused inframe with human CD3ζ transmembrane and cytoplasmic domains. These fusion fragments were then transferred into vectors from InVitrogen (Carlsbad, Calif.) for expression in mammalian cells, either pcDNA3.1(+) (G418 selection) for the β chain or pcDNA3.1(+)/hygro (hygromycin antibiotic resistance) for the α chain. The alternate drug resistance markers allowed for selection of the presence of both plasmids in transfected mammalian cells. The α9s1/hCD3ζ fragment cloned into pcDNA3.1(+)/hygro as a KpnI to NotI fragment. The a22s1/hCD3ζ fragment was cloned into pcDNA3.1(+)/hygro as a fragment containing a blunt end, made by cutting with the restriction enzyme SalI and filling in with the enzyme T4 polymerase, and a NotI end. The corresponding pcDNA3.1(+)/hygro vector was prepared by cutting with the restriction enzyme HindIII and filling in with T4 polymerase then cutting with NotI. The 12-4/hCD3z fragment was cloned into pcDNA3.1(+) as a HindIII to NotI fragment. The vectors resulting from these cloning steps are designated pTVW456-1, pTVW459-1 and pTVW457-1 and contain α9s1/hCD3ζ, α22s1/hCD3ζ and β12-4/hCD3ζ respectively.

TABLE 16

Oligonucleotide primers used in cloning TCR
α and β chains as CD3ζ fusions

| | |
|---|---|
| designation | sequence |

| | |
|---|---|
| VW535 | 5′-TAT GGT CGA CAA GCT TCA CAG AGG GCC TGG TCT GG-3′ |

(5′ primer for β 12-4, inserts
HindII and SalI site 47 nt preceding ATG in leader)

| | |
|---|---|
| VW537 | 5′-TAT GGT CGA CGG TAC CCC AGA AAA GAC CTC CAG AAA ATA GC-3′ |

(5′ primer for α 9s1, inserts
SalI and KpnI sites 49 nt preceding ATG in leader)

| | |
|---|---|
| VW536 | 5′-GGT GGT TAA CGT CTG CTC TAC CCC AGG CCT C-3′ |

(3′ primer for β constant, inserts
HpaI site just before 3^{rd} cysteine in constant region)

| | |
|---|---|
| VW539 | 5′-GGT GGT TAA CGG AAC TTT CTG GGC TGG GGA AGA AGG-3′ |

(3′ primer for α constant, inserts
HpaI site just before 3^{rd} cysteine in constant region)

| | |
|---|---|
| VW540 | 5′-TAT GGT CGA CGG TAC CCT TCA TGT TAA GGA TCA AGA CCA TTA TTT GG-3′ |

(5′ primer for a 22s1, inserts SalI and KpnI sites 45 nt preceding the
ATG in the leader.)

The human T cell line, J.RT3 (ATCC number TIB-153), selected for transfection with the TCR-hCD3ζ fusions is a derivative of the Jurkat lymphoblast line which does not express TCR P chain. These cells were transfected by electroporation with plasmids pTVW456-1, pTVW459-1 and pTVW457-1 singly or as α+β chain pairs, i.e. pTVW456-1+pTVW457-1 or pTVW459-1+pTVW457-1. Selection of the transfectants was accomplished using the appropriate antibiotic, G418 or hygromycin or both. Dual selection (using both antibiotics) took place in two stages. The cells were transfected and were allowed to grow in antibiotic-free medium for 1-2 days. Growth of the transfectants was then selected with media containing G418. After 7 days, hygromycin was added to the growth media. Expression of the TCR fusion molecules was evaluated by FACS analysis of cells stained with antibodies which detect either a monomorphic determinant of the human α/βchain of the T-cell receptor complex (clone BMA031, Beckman Coulter, Miami, Fla.) or the specific β chain variable region in the complex (clone 56C5, Beckman Coulter). These cells will be assayed for activation by DR2 restricted antigen presenting cells, D0208915 from Johns Hopkins Laboratories (Baltimore, Md.), which have been pulsed with the human MBP peptide containing amino acid residues 83-102 (amino acid sequence YDENPVVHFFKNIVTPRTPP) to confirm that the combination of α and β chains resulted in a functional TCR. The use of the recombinant T cells in screening assays is described in Example 13.

The following Examples pertain to various screening methodologies of the invention using MHC antigens consisting of MHC/peptide complexes.

### EXAMPLE 10

### Identification of Compounds that Inhibit TCR:MHC/Peptide Interactions Using a Cell-Based T Cell Activation Assay

A T cell-based screening assay was developed to detect inhibitors of the TCR:MHC/peptide interaction. Normally, T cells become activated when they come into contact with antigen presenting cells (APCs) bearing specific MHC/peptide complexes on their surfaces. For example, following incubation with IA^{d}-positive A20 APCs pulsed with OVA peptide, DO1.10 T cells become activated and produce IL-2. While interaction between a number of different cell surface receptors can contribute to level of the T cell response, the specific interactions between the DO11.10 TCR and the OVA/IA^{d} complex are required to initiate the response. To separate responses due solely to interaction between the TCR and MHC/peptide complex from those contributed to by other surface receptors, an assay method was developed for stimulating T cell responses with purified recombinant single-chain MHC/peptide complexes. This method can be used to monitor the ability of the T cell to respond to different MHC/peptide complexes as described previously (U.S. Ser. No. 09/204,979). This method is also particularly useful in detecting compounds that inhibit or stimulate the ability of the T cell to respond to a MHC/peptide complex.

Of particular interest is the detection of small molecular weight compounds, such as organic chemicals, peptides and nucleic acids, that inhibit or antagonize T cell responses to the MHC/peptide complex. More specifically, detection of small molecular weight compounds that antagonize the interaction between the TCR and the MHC/peptide complex is of great interest. Such compounds could have utility in the development of immunosuppressive drug candidates for treatment of immune disorders. Compounds that stimulate the ability of the T cell to respond to the MHC/peptide complex could have utility in the development of vaccine strategies.

To facilitate the analysis of a large number of compounds, a “dual” assay format was developed in which both stimulation of the T cells and measurement of the T cells responses can be carried out in a single well of a microtiter plate, as shown in FIG. 5B. In this assay, anti-cytokine mAb and the stimulating sc-MHC/peptide complex are immobilized on the well. The MHC/peptide-restricted T cells are added to the well together with the soluble test compound or compound diluent. Cytokines produced by the activated T cells are captured by the anti-cytokine antibody. Following an incubation period, the T cells and compounds are removed, the wells washed and secondary anti-cytokine mAb is added to allow detection of the amount of cytokine produced by the T cells. Compounds that inhibit T cell stimulation by the immobilized sc-MHC/peptide complex result in less cytokine production than is seen in wells receiving compound diluent.

For example, 50 ng of purified scIA^{d}/OVA protein and 100 ng of rat anti-mouse 1L-2 mAb (PharMingen part no. 18161D) were simultaneously coated in 50 μl PBS (Cellgro Catalog no. 21-031-cv) onto wells of a 96 well microtiter plate (Nunc Maxisorp). After 24 hours, the coat solution was removed and the wells received 100 μl media (IMDM with 10% FBS) with 2.5% dimethyl sulfoxide (DMSO Sigma part no. 2650) or 100 μl media containing the test compound in 2.5% DMSO. DO11.10 T cells (1×10⁵ cells in 100 μl media) were then added. Following an 8 hours incubation at 37° C. in 10% CO₂, the cells were removed and the wells were washed with Tris buffered saline with 1% Triton X-100. Biotinylated anti-IL-2 mAb (PharMingen Part no. 18172D) (100 ng in 100 μl PBS with 10% FBS) was added to each well. After overnight incubation at 4° C., the wells were washed and 250 ng avidin peroxidase (Sigma part no. A3151) in 100 μl 10% FBS-PBS was added to each well. After 30 min at 37° C., the wells were washed and 100 μl of TMB substrate (BioFX laboratories cat no. TMBW-0100-01) was added. Substrate reaction was stopped with 1M sulfuric acid and the absorbance of the TMB chromophore product was read at 450 nm. In this assay, the absorbance reading correlates with the amount of IL-2 produced by the cells.

By titering each of the assay components, a signal to noise ratio above 5 was achieved. Two thousand small molecules from a chemical library were tested for inhibition of IL-2 production in the above assay format. In each plate, multiple wells were run containing compound diluent (DMSO) to establish the amount of IL-2 produced by the scIA^{d}/OVA-stimulated DO11.10 T cells. Any compound that significantly lowered the amount of IL-2 produced, i.e. showed IL-2 absorbance readings in this assay more than 3 standard deviations lower than that seen in the diluent wells, was characterized further.

For example in assay plate 5-2, DO11.10 T cell stimulation was assayed in eight wells as described above using DMSO as the control compound diluent. The mean IL-2 absorbance reading for these wells was 2.198 with a standard deviation of 0.248. Forty test compounds were run in duplicate on this microtiter plate. Two compounds (5E1 and SF6) showed significantly lower IL-2 absorbance readings (1.256 and 0.136, respectively) than the control (mean-3SD=1.454) indicating that these compounds inhibited IL-2 production by the scIA^{d}/OVA-stimulated DO11.10 T cells. The mean IL-2 absorbance readings for wells receiving the other 38 compounds were not less than 1.454, indicating the range of the inhibitory responses. Of the 2000 compounds tested, 46 were repeatedly found to inhibit IL-2 production by the scIA^{d}/OVA-stimulated DO11.10 T cells in this assay format.

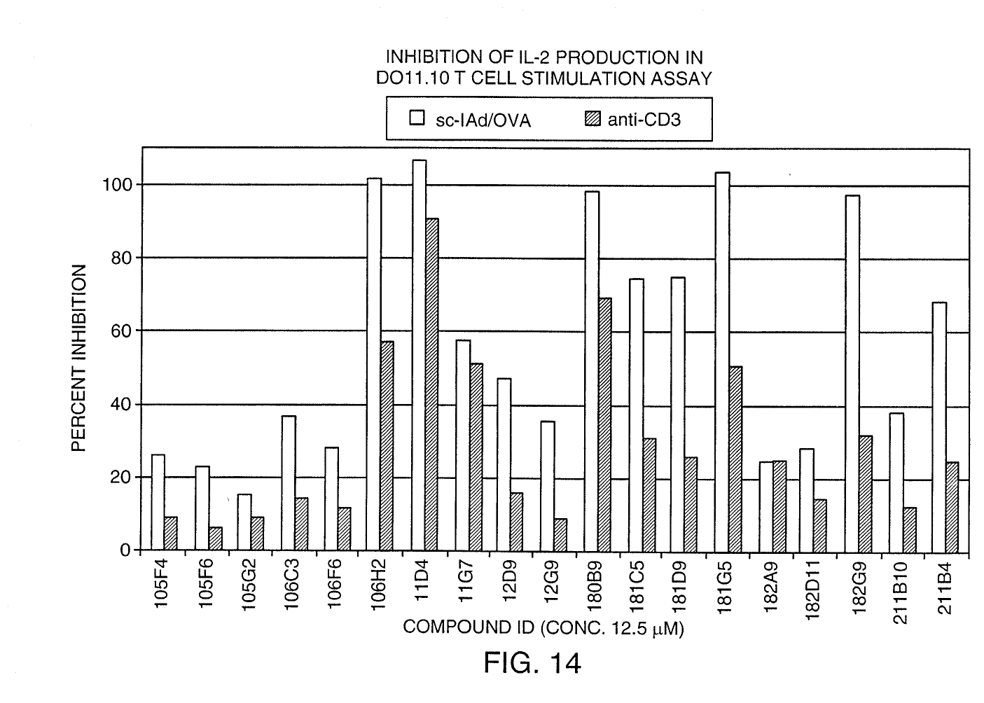

To determine if inhibition was due specifically to interference of scIA^{d}/OVA:DO10.10 TCR interactions and not due to other nonspecific sources of inhibition, the effects of the inhibitory compound on T cell stimulation by anti-CD3 mAb was assessed. Anti-CD3 antibody is able to bind to the TCR/CD3 complex at a different site than is recognized by the MHC/peptide complex. This antibody when immobilized on the well is able to stimulate IL-2 production by the DO11.10 T cell. For this assay, wells of a 96 well microtiter plate were coated simultaneously with 25 ng of anti-CD3 antibody (PharMingen part no. 0151D) and 100 ng of rat anti-mouse IL-2 mAb in 50 μl PBS. After 24 hours, the coat solution was removed and the wells received 100 μl media (IMDM with 10% FBS) with 2.5% dimethyl sulfoxide or 100 μl media containing the test compound in 2.5% DMSO. DO11.10 T cells (1×10⁵ cells in 100 μl media) were then added. Following an 8 hours incubation at 37° C. in 10% CO₂, the cells were removed and the wells were washed with Tris buffered saline with 1% Triton X-100. Biotinylated anti-IL-2 mAb (100 ng in 100 μl PBS with 10% FBS) was added to each well. After overnight incubation at 4° C., the wells were washed and 250 ng avidin peroxidase in 100 1 10% FBS-PBS was added to each well. After 30 min at 37° C., the wells were washed and 100 μl of TMB substrate was added. Substrate reaction was stopped with 1M sulfuric acid and the absorbance of the TMB product was read at 450 nm. Representative assay results are shown in FIG. 14. Thirty compounds that inhibited scIA^{d}/OVA-mediated stimulation were found to have insignificant inhibition of IL-2 production in the anti-CD3 assay. These results indicate that although MHC/peptide-dependent stimulation was inhibited, these compounds do not affect the ability of the T cells to respond to stimulation through the TCR/CD3 complex. Therefore, it appears unlikely that these compounds act through inhibiting intercellular mechanisms leading to IL-2 production or release.

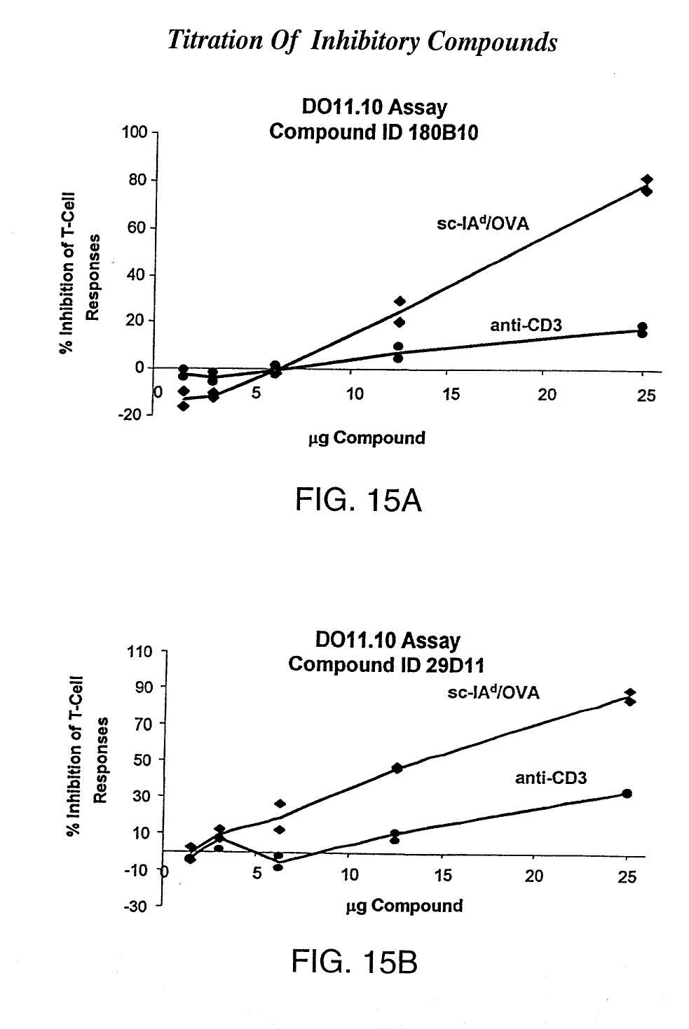

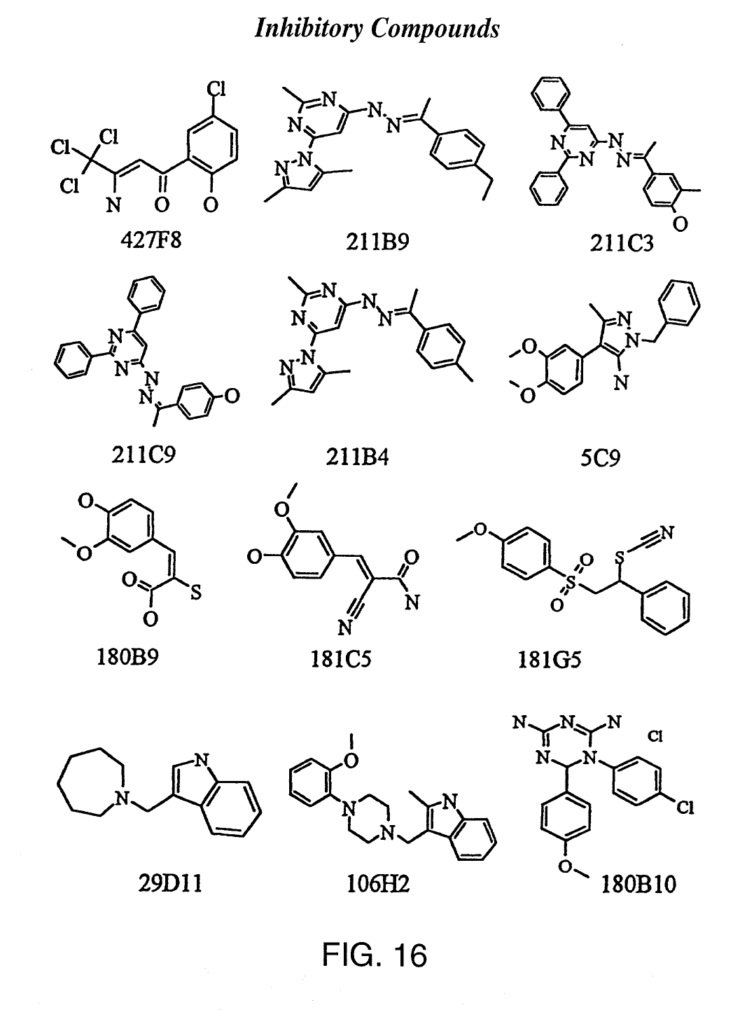

The thirty inhibitory compounds were then titered out in the scIA^{d}/OVA and the anti-CD3 Ab assays described above and showed dose dependent inhibition of scIA^{d}/OVA-mediated stimulation. Examples of these titration curves are shown in FIGS. 15A and 15B and representatives of the thirty inhibitory compounds are shown in FIG. 16.

To determine if inhibition was specific to the interaction with the OVA peptide in the scIA^{d}/OVA complex, the effects of the inhibitory compounds on T cell stimulation by scIA^{d}/GD was assessed. It has previously been shown that immobilized scIA d/GD could stimulate the GD12 T cell hybridoma to produced IL-2. To test whether the inhibitory compounds also had an effect on the GD12 cells, 100 ng of purified scIA^{d}/GD protein and 100 ng of rat anti-mouse IL-2 mAb were simultaneously coated in 50 μl PBS onto wells of a 96 well microtiter plate. After 24 hours, the coat solution was removed and the wells received 100 μl media (IMDM with 10% FBS) with 2.5% dimethyl sulfoxide or 100 μl media containing the test compound in 2.5% DMSO. GD12 T cells (1×10⁵ cells in 100 μl media) were then added. Following an 8 hours incubation at 37° C. in 10% CO₂, the cells were removed and the wells were washed with Tris buffered saline with 1% Triton X-100. Biotinylated anti-IL-2 mAb (100 ng in 100 μl PBS with 10% FBS) was added to each well. After overnight incubation at 4° C., the wells were washed and 250 ng avidin peroxidase in 100 μl 10% FBS-PBS was added to each well. After 30 min at 15=37° C., the wells were washed and 100 μl of TMB substrate was added. Substrate reaction was stopped with 1M sulfuric acid and the absorbance was read at 450 nm. All of the compounds that inhibited scIA^{d}/OVA-mediated DO11.10 stimulation also inhibited IL-2 production by the sc-IA^{d}/GD stimulated GD12 cells, indicating that the compounds do not inhibit stimulation in a peptide-specific manner.

The specificity of the inhibitory compounds was examined further using a different MHC/peptide:T cell combination. As described in Example 1, the IE^{k}/PCC was constructed as a single chain MHC class II molecule and found to activate 2B4 T cells to secrete IL-2. A dual format assay was developed as above using 2B4 cells and the scIE^{k}/PCC protein. In this assay, 200 ng of purified scIE^{k}/PCC protein and 100 ng of rat anti-mouse IL-2 mAb were simultaneously coated in 50 μl PBS onto wells of a 96 well microtiter plate. After 24 hours, the coat solution was removed and the wells received 100 μl media (IMDM with 10% FBS) with 2.5% dimethyl sulfoxide or 100 μl media containing the test compound in 2.5% DMSO. 2B4 T cells (1×10⁵ cells in 100 μl media) were then added. Following an 8 hours incubation at 37° C. in 10% CO₂, the cells were removed and the wells were washed with Tris buffered saline with 1% Triton X-100. Biotinylated anti-IL-2 mAb (100 ng in 100 μl PBS with 10% FBS) was added to each well. After overnight incubation at 4° C., the wells were washed and 250 ng avidin peroxidase in 100 μl 10% FBS-PBS was added to each well. After 30 min at 37° C., the wells were washed and 100 μl of TMB substrate was added. Substrate reaction was stopped with 1M sulfuric acid and the absorbance was read at 450 nm. Twenty-two small molecules that showed greater inhibition of IL-2 secretion with immobilized scIA^{d}/OVA compared that of the anti-CD3 Ab were tested in the scIE^{k}/PCC:2B4 assay. One small molecule (427-F8) showed greater inhibition of the scIA^{d}/OVA:DO11.10 T cell interaction than the scIE^{k}/PCC:2B4 T cell interaction as measured by IL-2 secretion. A typical titration curve showing percent inhibition at multiple doses of 427-F8 in both assays is shown in Table 17. Similar results were observed in three independent assays. The results suggest that the test compounds can exhibit some degree of specificity for inhibiting TCR:MHC/peptide interactions.

TABLE 17
Selective Activity of Compound 427-F8 in
Inhibiting IL-2 Secretion by MHC/peptide-Stimulated T Cells

% Inhibition of IL-2 Secretion
Assay Format

| | scIA^{d}/OVA: | scIE^{k}/PCC: |
|---|---|---|
| 427-F8 Conc. (μM) | DO11.10 T cells | 2B4 T cells |
| 16.6 | 83 | 86 |
| 5.5 | 85 | 86 |
| 1.8 | 85 | 83 |
| 0.6 | 57 | 11 |
| 0.2 | 0 | 4 |
| 0.06 | 0 | 3 |

The activity of the inhibitory compounds was examined further in stimulation assays in which antigen presenting cells (APC) carrying the appropriate MHC on their surface are loaded with exogenous peptide and used to stimulate T cell responses in vitro. In this assay, 100 ng of anti-IL2 antibody was coated onto a well of a 96 well microtiter plate. After 24 hours, the coat solution was removed and the wells received 2×10⁵ A20 cells (APCs carrying IA^{d} molecules) in 100 μl media (IMDM with 10% FBS), 5 μg of OVA peptide in 10 μl of media, 1×10⁵ DO11.10 T cells in 100 μl of media, and 5 μl of the test compound in PBS. After 3 hrs at 37° C. in a 10% CO₂ incubator, the cells were removed and the wells were washed with Tris buffered saline with 1% Triton X-100. Biotinylated anti-IL-2 mAb (PharMingen Part no. 18172D) (100 ng in 100 μl PBS with 10% FBS) was added to each well. After 1 hr at 37° C., the wells were washed and 250 ng avidin peroxidase (Sigma part no. A3151) in 100 μl 10% FBS-PBS was added to each well. After 30 min at 37° C., the wells were washed and 100 μl of ABTS substrate (BioFX laboratories) was added. Substrate reaction was developed and the absorbance was read at 405 nm. The absorbance reading correlate with the ability of the OVA peptide-loaded IA^{d} molecules on the A20 cells to interact with the DO11.10 TCR and stimulate IL-2 production. The ability of a compound to antagonize interactions between the MHC/peptide complex and TCR is measured as a decrease in the amount absorbance read at 405 nm. Typical results of these studies for candidate inhibitory compounds are shown in Table 18 and indicate that the compounds are able to block the ability of the peptide-loaded APC to stimulate T cell responses.

TABLE 18
Inhibitory effects of test compounds on DO11.10 T cell
stimulation by OVA peptide-loaded A20 cells
IL-2 ELISA Values

| 211-C3 | | 211-C9 | | 12G9 | | 211B9 | |
|---|---|---|---|---|---|---|---|
| μM | A-405 | μM | A-405 | μM | A-405 | μM | A-405 |
| 0 | 2.024 | 0 | 2.024 | 0 | 1.906 | 0 | 1.920 |
| 2.3 | 2.128 | 1 | 2.101 | 1.5 | 2.047 | 1.8 | 1.608 |
| 7 | 1.984 | 3 | 1.947 | 5 | 1.940 | 5.5 | 1.154 |
| 20 | 1.437 | 10 | 1.289 | 14 | 1.795 | 16.7 | 1.127 |
| 62 | 0.262 | 31 | 0.235 | 42 | 1.630 | 50 | 0.944 |

In summary, a practical method has been developed to screen for substances that will interfere with TCR:MHC antigen interactions.

### EXAMPLE 11

### Development of Screening Assays Using T Cell Hybridomas Engineered to Express Surface D011.10 scTCR-CD3ζ Fusion Protein.

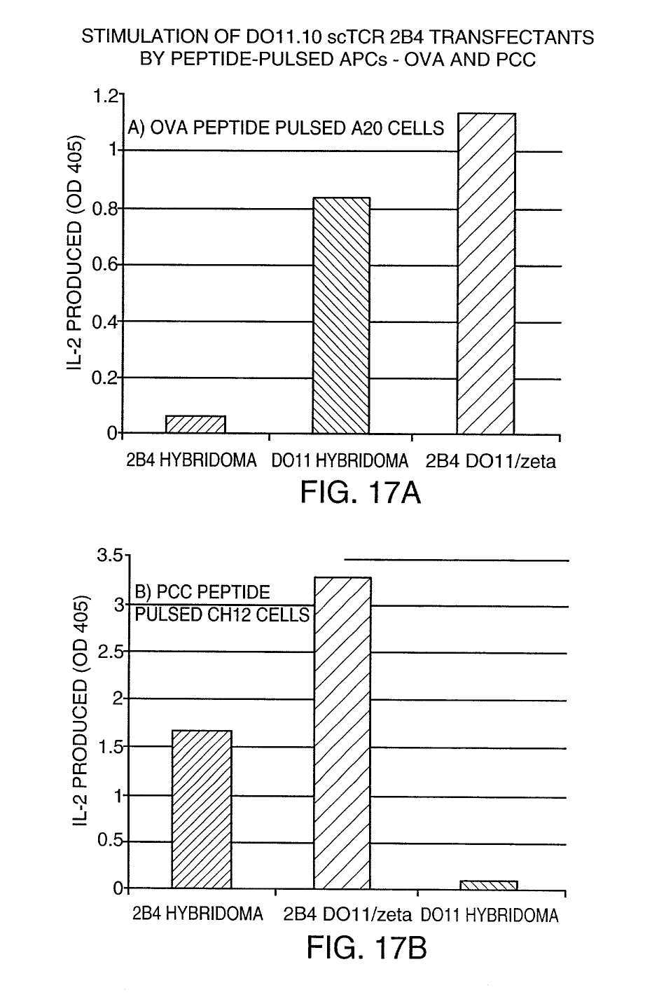

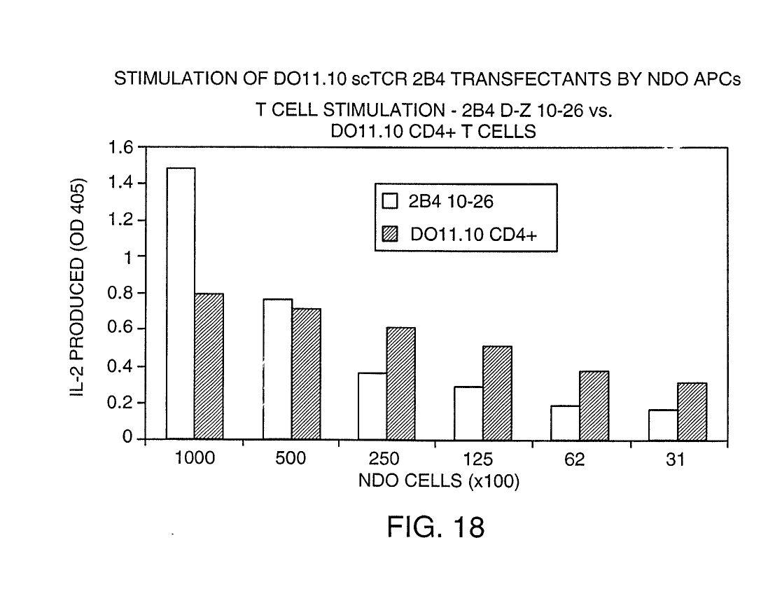

The objective was to further develop a cell-protein based approach for identifying agonists and antagonists capable of interfering with the interaction between the TCR and MHC antigen molecules. As described in this example, an assay format was developed using an immortalized T cell line expressing a recombinant single-chain T cell receptor. The advantage of this approach is that a sufficient amount of cells can be generated to conduct the screening of a large number of test compounds. As described in Example 7, the 2B4 murine T cell hybridoma was used to create a novel cell line that expresses on its surface the DO11.10 TCR as a three-domain scTCR-CD3ζ fusion protein. Specific stimulation was observed for 2B4 T cells transfected with DNA encoding for DO11.10 scTCR-CD3ζ after incubating cells with A20 cells pulsed with OVA peptide, as shown in FIG. 17A. Furthermore, these same cells were not stimulated when exposed to A20 cells pulsed with GD peptide. In another experiment using NSO myeloma cells expressing surface bound scIA^{d}/OVA (NDO cells), stimulation of the DO11.10 scTCR transfected T hybridoma cells was observed, as shown in FIG. 18. The wild-type 2B4 T cells (expressing the 2B4 TCR on their surface) incubated in the presence of the NDO cells were non-responsive. These data support the claim that scTCR-CD3ζ fusions can be used to reconstitute T cells with new antigen specificity.

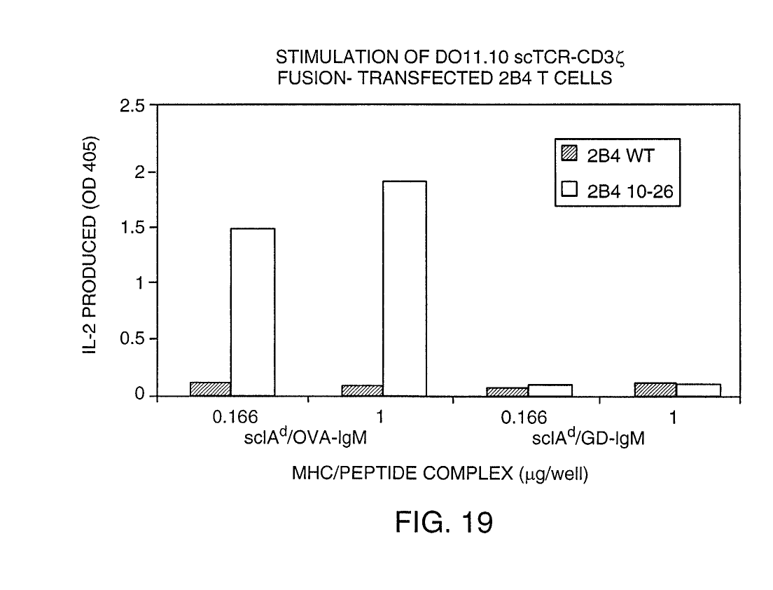

As discussed above, an ideal approach to screening the chemical libraries would be to develop a cell line that could be stimulated by purified recombinant MHC/peptide complexes. To test the concept of using purified MHC/peptide molecules to stimulate a T cell line engineered to express surface scTCR-CD3ζ fusion protein, wells were coated with either 1000 or 160 ng of scIA^{d}/OVA-IgM fusion protein and the appropriate T cells were added. Cell stimulation was measured by assaying for IL-2 protein secreted into the culture supernatant. The detection of IL-2 was determined using a pair of anti-murine IL-2 antibodies purchased from PharMingen and were used in a sandwich ELISA format as described previously (Rhode et al. 1996. J. Immunol. 157: 4885). Control wells were coated at the same concentrations of protein except the recombinant scIA^{d}/GD-IgM protein was used. After incubating 10⁵ 2B4 cells or 2B4 transfectants in the wells for 14 hours at 37° C. in the presence of 5% CO₂, 100 μl of supernatant was collected and assayed for IL-2. FIG. 19 shows the results from one experiment. As expected, control wells coated with scIA d/gD-IgM could not stimulate either the DO11.10 scTCR 2B4 transfectant or the wild-type 2B4 T cells. More importantly, only the transfectants produced IL-2 when presented with immobilized scIA^{d}/OVA-IgM protein, in contrast, the same protein did not stimulate the wild-type 2B4 T cells. These results show that T cells expressing recombinant DO11.10 scTCR-CD3ζ on their surface can be efficiently stimulated in the presence of immobilized IA^{d}/OVA complexes, but not by scIA^{d}/GD-IgM complexes.

To further develop a screening assay using the transfected cell line, the dual format assay was set-up using varying concentrations of immobilized scIA^{d} molecules. The assay conditions were identical to those described in Example 10. The results from this assay are shown in Table 19. These results indicate that both the scIA^{d}/OVA and the scIA^{d}/OVA-IgM are capable of stimulating the DO11.10 scTCR transfectants. As was observed previously, immobilized scIA d/OVA-IgM molecules were more potent in stimulating the response than immobilized scIA^{d}/OVA molecules. The peptide specificity of the transfectants was also observed. The use of the IgM format for the MHC antigen complex shows how the use of multi-valent formats can increase the sensitivity of the assay.

TABLE 19
Activation of DO11.10 scTCR 2B4 Transfectants by
scIA^{d}/OVA complexes

| | IL-2 ELISA Values (A₄₀₅) | | | |
|---|---|---|---|---|
| ng complex/ | | | | scIA^{d}/ |
| well | scIA^{d}/OVA-IgM | scIA^{d}/GD-IgM | scIA^{d}/OVA | GD |
| 250 | 2.274 | 0.224 | 1.063 | 0.227 |
| 125 | 2.325 | 0.233 | 1.059 | 0.179 |
| 62 | 1.822 | 0.224 | 1.032 | 0.164 |
| 31 | 1.324 | 0.226 | 0.646 | 0.213 |
| 15 | 0.273 | 0.199 | 0.397 | 0.152 |
| 7 | 0.264 | 0.213 | 0.236 | 0.163 |
| 3 | 0.234 | 0.190 | | |
| 1 | 0.214 | 0.196 | | |

Based on the results shown above, a screening method could be developed using a TCR transfected T cell line and immobilized MHC antigen molecules. For example, purified scIA^{d}/OVA-IgM protein and rat anti-mouse IL-2 mAb are simultaneously coated in 50 μl PBS onto wells of a 96 well microtiter. After 24 hours, the coat solution is removed and the wells receive 100 μl media (IMDM with 10% FBS) with 2.5% dimethyl sulfoxide or 100 μl media containing the test compound in 2.5% DMSO. The DO11.10 scTCR 2B4 transfectants (1×10⁵ cells in 100 μl media) are then added. Following an 8 hours incubation at 37° C. in 10% CO₂, the cells are removed and the wells are washed with Tris buffered saline with 1% Triton X-100. Biotinylated anti-IL-2 mAb (100 ng in 100 μl PBS with 10% FBS) is added to each well. After overnight incubation at 4° C., the wells are washed and 250 ng avidin peroxidase in 100 μl 10% FBS-PBS is added to each well. After 30 min at 37° C., the wells are washed and 100 μl of TMB substrate is added. Substrate reaction will be stopped with 0.18 M sulfuric acid and the absorbance of the reaction product is read at 450 nm. The ability of a compound to antagonize interactions between the MHC/peptide complex and the recombinant TCR will be measured as a decrease in the amount of cytokine secreted into the culture media by the T cell transfectants. Assays in which anti-TCR Ab is used instead of the MHC/peptide complex to stimulate the T cells will be carried out to determine whether the inhibitory compounds act by antagonizing TCR:MHC/peptide complex interactions or through a different mechanism. Compounds that inhibit T cell stimulation by the MHC/peptide complex but do not inhibit stimulation by the anti-TCR antibody will be pursued as lead compounds.

### EXAMPLE 12

### Development of Screening Assays Using T Cell Hybridomas Engineered to Express Surface D011.10 scTCR-CD3ζ Fusion Protein and Recombinant CD4 Receptors.

The next goal was to further refine the cell-based screening approach. Recombinant T cells were engineered to express a single-chain TCR-CD3ζ fusion and the CD4 receptor on the cell surface. These manipulations allow these cells to be stimulated by MHC/peptide complex. The advantages to this approach are that the cells can be designed and optimized to suit the particular assay parameters. For example, cells expressing high levels of recombinant scTCR and/or CD4 receptors may show greater sensitivity to stimulation by MHC/peptide complexes. As described in Example 8, stable BWDZ-CD4⁺ T cell transfectants were generated that expressed both DO11.10 scTCR-CD3ζ fusion protein and recombinant CD4 receptor on the cell surface. To test the concept of using purified MHC/peptide molecules to stimulate the BWDZ:CD4⁺ T cell line, wells were coated with varying concentrations of scIA^{d}/OVA-IgM fusion protein. The wells were also coated with rat anti-mouse IL-2 antibody as described in Example 10. The BWDZ:CD4⁺ T cells were then added and T cell stimulation was measured by assaying for IL-2 secreted into the culture supernatant as described in Example 10. Control wells were coated at the same concentrations of protein except the recombinant scIA^{d}/GD-IgM protein was used. The results from such an assay are shown in Table 20. The results indicate that the recombinant BWDZ:CD4⁺ T cells expressing surface DO11.10 scTCR and CD4 receptor can be specifically stimulated by immobilized scIA^{d}/OVA-IgM and that a screening method could be developed using recombinant T cell lines and immobilized MHC/peptide molecules.

TABLE 20
Activation of BWDZ: CD4+ Transfectants by
scIA^{d}/OVA-IgM complexes

IL-2 ELISA Values (A₄₀₅)

| ng complex/well | scIA^{d}/OVA-IgM | scIA^{d}/GD-IgM |
|---|---|---|
| 1000 | 1.277 | 0.375 |
| 200 | 0.340 | 0.309 |
| 40 | 0.305 | 0.282 |

For example, purified scIA^{d}/OVA-IgM protein and rat anti-mouse IL-2 mAb are simultaneously coated in 50 μl PBS onto wells of a 96 well microtiter. After 24 hours, the coat solution is removed and the wells receive 100 μl media (IMDM with 10% FBS) with 2.5% dimethyl sulfoxide or 100 μl media containing the test compound in 2.5% DMSO. The BWDZ:CD4+transfectants (1×10⁵ cells in 100 μl media) are then added. Following an 8 hours incubation at 37° C. in 10% CO₂, the cells are removed and the wells are washed with Tris buffered saline with 1% Triton X-100. Biotinylated anti-IL-2 mAb (100 ng in 100 μl PBS with 10% FBS) is added to each well. After overnight incubation at 4° C., the wells are washed and 250 ng avidin peroxidase in 100 μl 10% FBS-PBS is added to each well. After 30 min at 37° C., the wells are washed and 100 μl of TMB substrate added. Substrate reaction is stopped with 0.18 M sulfuric acid and the absorbance read at 450 nm. The ability of a compound to antagonize interactions between the MHC/peptide complex and the recombinant TCR will be measured as a decrease in the amount of cytokine secreted into the culture media by the T cell transfectants. Assays in which anti-TCR Ab is used instead of the MHC/peptide complex to stimulate the T cells will be carried out to determine whether the inhibitory compounds act by antagonizing TCR:MHC/peptide complex interactions or through a different mechanism. Compounds that inhibit T cell stimulation by the MHC/peptide complex but do not inhibit stimulation by the anti-TCR antibody will be pursued as lead compounds.

### EXAMPLE 13

### Development of Cell-Based Screening Assays to Detect Inhibitors of Autoimmune TCR:MHC/Peptide Interactions.

Our objective was to further develop a cell-protein based approach for identifying antagonists capable of interfering with the interaction between the TCR and MHC antigen molecules associated with human autoimmune diseases. As described in this example, an assay format was devised using an immortalized T cell line expressing a recombinant T cell receptor derived from T cells isolated from a multiple sclerosis patient. Human T cell lines or clones are very difficult to grow in large amounts in culture and after extended growth often lose specificity for their antigens. The advantage of the recombinant T cell approach is that a sufficient amount of cells can be generated to conduct the screening of a large number of test compounds. As described in Example 9, the TCR α and β chain genes were cloned from the MBP-restricted human MS T cell line, E11. The expression vectors capable of expressing the extracellular domains of TCR α or β chains fused inframe with human CD3ζ, transmembrane and cytoplasmic domains were generated and transfected into immortalized T cells. The transfected cells will be used in stimulation assays with MBP peptide-loaded DR2+APCs. The E11 TCR transfected cell line can then be used to develop screening assays with immobilized scDR2/MBP protein.

For example, purified scDR2/MBP-IgG protein (see Example 4) and anti-human IL-2 mAb are simultaneously coated in 50 μl PBS onto wells of a 96 well microtiter. The amount of scDR2/MBP-IgG required to generate a linear response from the recombinant T cell will be determined experimentally. After 24 hours, the coat solution is removed and the wells receive 100 μl media (IMDM with 10% FBS) with 2.5% dimethyl sulfoxide or 100 μl media containing the test compound in 2.5% DMSO. The E11 TCR T cell transfectants (1×10⁵ cells in 100 μl media) are then added. Following an 8 hours incubation at 37° C. in 10% CO₂, the cells are removed and the wells washed with Tris buffered saline with 1% Triton X-100. Biotinylated anti-IL-2 mAb (100 ng in 100 μl PBS with 10% FBS) is added to each well. After overnight incubation at 4° C., the wells are washed and 250 ng avidin peroxidase in 100 μl 10% FBS-PBS is added to each well. After 30 min at 37° C., the wells are washed and 100 μl of TMB substrate added. Substrate reaction is stopped with 0.18 M sulfuric acid and the absorbance read at 450 nm. The ability of a compound to antagonize interactions between the MHC/peptide complex and the recombinant TCR will be measured as a decrease in the amount of cytokine secreted into the culture media by the T cell transfectants. Assays in which anti-TCR Ab is used instead of the MHC/peptide complex to stimulate the T cells will be carried out to determine whether the inhibitory compounds act by antagonizing TCR:MHC/peptide complex interactions or through a different mechanism. Compounds that inhibit T cell stimulation by the MHC/peptide complex but do not inhibit stimulation by the anti-TCR antibody will be pursued as lead compounds.

### EXAMPLE 14

### Development of a Model Cell-Based Screening Method for Identifying Compounds that Modulate TCR:MHC Antigen Interactions.

This example describes a model cell-based screening system for screening chemical libraries for agonists and antagonists capable of specifically modulating TCR interactions with the MHC antigen molecule. This model is based on an immortalized T cell line (e.g. T cell hybridoma) to express two or more different antigen specific T cell receptors on its surface. For example, the T cell could express its endogenous TCR and also a recombinant single-chain TCR of unique antigen specificity. The objective of the screening assay would be to identify compounds that show a differential effect in agonizing or antagonizing TCR:MHC/peptide interactions for one of the TCRs compared to the other TCR. These cells would be used in a plate assay with wells pre-coated with immobilized MHC/peptide molecules described above (e.g. single chain monomer, IgG or IgM). The MHC/peptide would stimulate IL-2 secretion by the T cell. Compounds would be screened for their ability to augment or inhibit IL-2 secretion by the T cells. Because of the generally weak interaction between TCR and MHC/peptide complex and the many steps in the IL-2 production/secretion process, one would predict the identification of many compounds that interfere (specifically and non-specifically) with T cell stimulation as evidenced by a decrease in IL-2 production. The use of a T cell that expresses two TCRs specific for unique targets would facilitate the screening process by providing an internal control for compounds that inhibit T cell stimulation by non-specific (TCR:MHC/peptide independent) mechanisms. For example, many of the compounds to be screened will probably block IL-2 production at points along the signaling pathway after TCR triggering and not necessarily at the point of TCR interaction with MHC/peptide. Compounds with general cytotoxic effects may also block IL-2 production. By testing stimulation with different MHC/peptide complexes, the dual TCR cell line will be extremely useful in determining whether the inhibitory compound acts by interfering with TCR:MHC/peptide interaction or at some other point in the IL-2 production/secretion process.

For this example, a model cell line expressing the endogenous 2B4 TCR and a recombinant DO11.10 scTCR was generated as described in Example 7. To evaluate the responsiveness of the T cell line to stimulation through the 2B4 and DO11.10 TCR, the cells were incubated in wells containing peptide pulsed antigen presenting cells (APC). For example, 2B4 transfectants were incubated with A20 (IA^{d}) cells pulsed with either OVA or PCC or with CH12 (IE^{k}) cells pulsed with OVA or PCC. The results of these experiments (FIG. 17A and FIG. 17B) showed that the 2B4 transfectants and wild-type 2B4 cells responded to CH12 cells pulsed with PCC but did not respond to CH12 cells pulsed with OVA or to A20 pulsed with PCC. However, in the presence of A20 cells pulsed with OVA, the 2B4 transfectants responded and produced IL-2 whereas the wild-type 2B4 cells were non-responsive. These data suggest that a T cell can be engineered to express at least two functionally distinct antigen specific TCRs.

The next experiment was to test these 2B4 transfectants for responsiveness to immobilized MHC/peptide molecules. The rationale for running these studies are explained in Example 10 and the results are shown in Example 11.

A model assay system will be developed to identify compounds that block the DO11.10 scTCR interaction with immobilized scIA^{d}/OVA. Assays using the DO11.10 scTCR 2B4 transfectants will be carried out as described in Example 11. The compounds that can inhibit stimulation of cells by blocking the DO11.10 TCR interaction with IA^{d}/OVA will then be screened in stimulation assays using the same DO11.10 scTCR 2B4 transfectants and immobilized scIE^{k}/PCC. If 2B4 TCR engagement with scIE^{k}/PCC resulted in cell stimulation, the inhibitor compound probably acts by targeting specific blocking of the DO11.10 TCR interaction with scIA^{d}/OVA. However, if inhibition of cell stimulation was found to be independent of the MHC/peptide complex used, the compound would probably be classified as a non-specific inhibitor of T cell stimulation.

The long-term objective will be to create a cell line reconstituted with two or more TCRs restricted by different MHC families. The advantage to this approach is that the variability of handling and running multiple cell lines within each screening experiment is eliminated.

### EXAMPLE 15

### Development of Cell Surface Receptor-Based Detection Assays for Identifying Inhibitors of TCR:MHC/Peptide Interactions

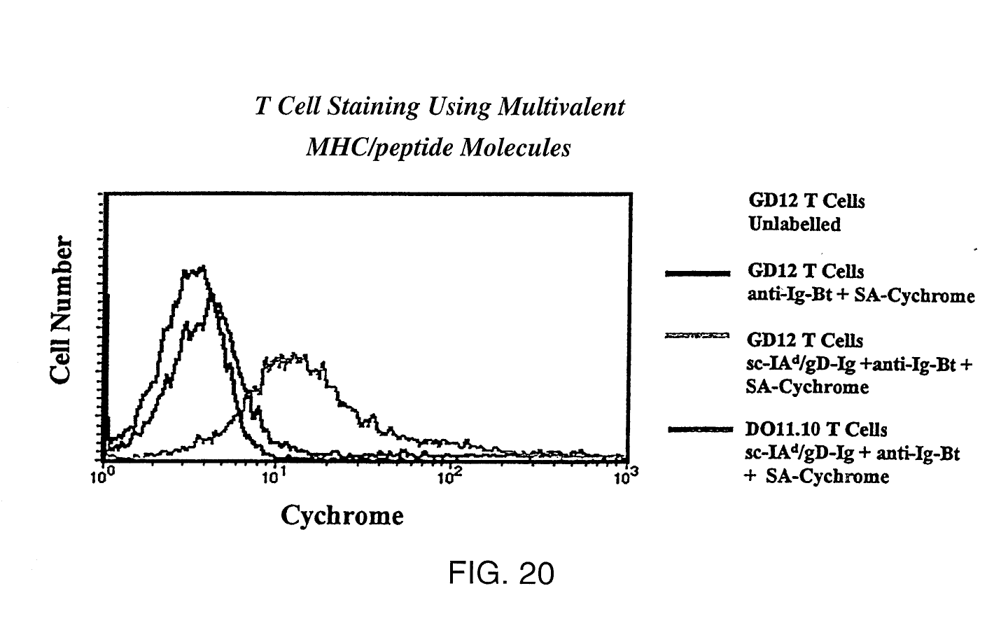

Multi-valent MHC/peptide complexes and TCRs have been used to fluorescently stain cells bearing the cognate receptors on their surfaces (Altman J. D. et al. (1996) Science 274:94-96). For example, T cell hydridomas can be specifically stained using multimeric sc-class II-Ig molecules. In this study, GD12 or DO11.10 T cell hybridomas (5×10⁵ cells/well) were incubated overnight at 37° C. with 10 μg/well scIA^{d}/GD-Ig molecules. The GD12 TCR is specific to the gD 246-261 peptide in the context of IA^{d}, whereas the DO11.10 TCR is specific to the OVA 323-339 peptide in the context of IA^{d}. Following the overnight incubation, the cells were washed with PBS and incubated with anti-mouse IgG2b mAb-biotin and streptavidin-Cychrome for 1 hour at 4° C. The cells were then washed with PBS, PBS containing 1% formaldehyde, and PBS. Flow cytometry of the stained cells indicated that the scIA^{d}/GD-Ig molecules specifically stained the GD12 T cell hybridoma but failed to stain the DO1.10 T cells (FIG. 20). No cell staining was observed in GD12 T cells incubated with anti-mouse IgG2b mAb-biotin and streptavidin-Cychrome alone. These results demonstrate that this assay can detect cell-surface TCRs using multi-valent MHC/peptide complexes as probes.

Simple plate-based cell ELISAs for detecting antigens on the surface of T cells have also been described in the literature (See Grunow, R. et al. (1994) J Immunol. Methods 171:93-102). While these assays detected binding of monoclonal antibodies, similar assay formats could be developed to detect compounds that inhibited interaction of the multi-valent MHC/peptide complex with the cell-surface TCR. For example, GD12 T cells are added in growth media to poly-lysine coated 96 well microtiter plates. The plates are incubated at 37° C. and 10% CO₂ to allow the T cell hydridomas to adhere to the coated wells. The multi-valent scIA^{d}/GD-Ig molecules are added to the cells with the test compound (or compound diluent as a control). Following incubation, the unbound scIA^{d}/GD-Ig molecules are removed and the adherent cells washed. A variety of commercially available probes, including enzyme-linked and fluorochrome linked antibodies, could be used to disclose the interaction between the scIA^{d}/GD-Ig molecules and the GD12 TCR. For example, an anti-IgG2b mAb-HPR probe can be added to detect the TCR-bound scIA^{d}/GD-Ig molecules. Unbound antibody is removed and the wells are incubated with TMB substrate to detect the TCR:scIA^{d}/GD-Ig:anti-IgG2b mAb-HPR complex. After development, the reactions are stopped with 1M sulfuric acid and the absorbance is determined at 450 nm. Initial studies will determine the appropriate number of T cells and the amounts of multi-valent sc-class II-Ig molecules and disclosing antibody probe required for sensitive and linear detection of the MHC/peptide-TCR interactions. Controls will be run in which a different MHC multimer, such as scIA^{d}/OVA-1 g, is added instead of the scIA^{d}/GD-Ig to establish the assay background. The ability of the test compound to inhibit specific MHC/peptide-TCR interactions will be measured as a loss of signal. For example, a test compound that inhibits interaction between the cell-surface GD 12 TCR and scIA^{d}/GD-Ig multimer would result in lower absorbance readings than observed in control wells containing compound diluent with the cells and MHC/peptide probe. The specificity of the inhibitory compounds could then be examined in other cell-based ELISAs using cells bearing different TCRs and their corresponding multi-valent MHC/peptide probes.

### EXAMPLE 16

### Development of Protein-Based Screening Methods for Identifying Compounds that Augment and Inhibit TCR:MHC Antigen Interactions.

The objective of this example was to develop a protein-protein binding assay using recombinant TCR and MHC antigen reagents. By establishing a protein-based assay, a high throughput screen could be developed to identify compounds that directly agonize or antagonize the target TCR:MHC/peptide interaction. The advantage to such a screening assay compared to the cell-based assay would be the reduction in the number of “false positive” compounds detected that are cytotoxic or nonspecifically affect cellular responses. The approach taken was to establish an ELISA format using purified recombinant TCR and MHC antigen complexes.

Briefly, cDNA encoding the 264 TCR (peptide fragment 264-272 from wild-type human tumor suppressor protein, p53 and restricted by HLA-A2) was provided by Dr. Linda Sherman (Scripps Research Institute, La Jolla, Calif.). Amplification of the Vα3.1 and the Vβ3.0 genes was carried out as described in the pending U.S. patent application Ser. No. 08/813,731 (Fusion Proteins Comprising Bacteriophage Coat Protein and A Single-Chain T cell Receptor” and Ser. No. 08/943,086 (“Fusion Proteins Comprising Single-Chain T Cell Receptor and an Immunoglobulin Light Chain Constant Region”) except that the oligonucleotides used were specific for the 264 TCR. The cloning of the 264 TCR as a three-domain scTCR-C kappa fusion protein was carried out in the same manner as described for the DO11.10 TCR (as described in the pending U.S. patent applications—see above). The 264 scTCR-C kappa fusion vector was transfected into CHO cells for expression of soluble protein in the culture media. The 264 scTCR protein was then purified to homogeneity on an affinity column as confirmed by SDS-PAGE analysis.

Separately, a vector system used to produce the HLA-A2 and beta-2 microglobulin molecules was provided by Dr. John Altman (Emory University, Atlanta, Ga.) and has been described in Altman et al. (Altman, J. D., P. A. H. Moss, P. J. R. Goulder, D. H. Barouch, M. G. McHeyzer-Williams, J. I. Bell, A. J. McMichael & M. M. Davis. 1996. Science 274:94-96). To produce HLA-A2 molecules loaded with the peptide of interest, a protocol by Dr. Mark Davis (Stanford University, Palo Alto, Calif.) (see Altman et al. 1996. Science 274:94-96) was followed that resulted in the formation of functional molecules. Correctly folded peptide/HLA-A2 complex was isolated on a Sephadex-200 size exclusion column as a single peak migrating at 50 kD. The protein complex was then biotinylated using biotin ligase (Avidity, Denver, Colo.) to attach a single biotin moiety to a C-terminal BirA sequence. Biotinylated A2 complexes were multimerized using streptavidin, a biotin specific protein capable of binding up to four biotin residues per molecule of streptavidin. Using the HLA-A2 and the streptavidin protein at a molar ratio of 4:1 respectively, tetrameric HLA-A2/peptide complexes were generated. The advantage of using the tetramer (compared to the monomer) is the greater binding avidity displayed by the tetramer when interacting with TCR. In support of this, protein binding studies using BIAcore instrument showed that the soluble tetrameric HLA-A2/264 peptide complexes bound to immobilized 264 scTCR-C kappa fusion protein to a greater extent than the monomeric HLA-A2/264 peptide complexes.

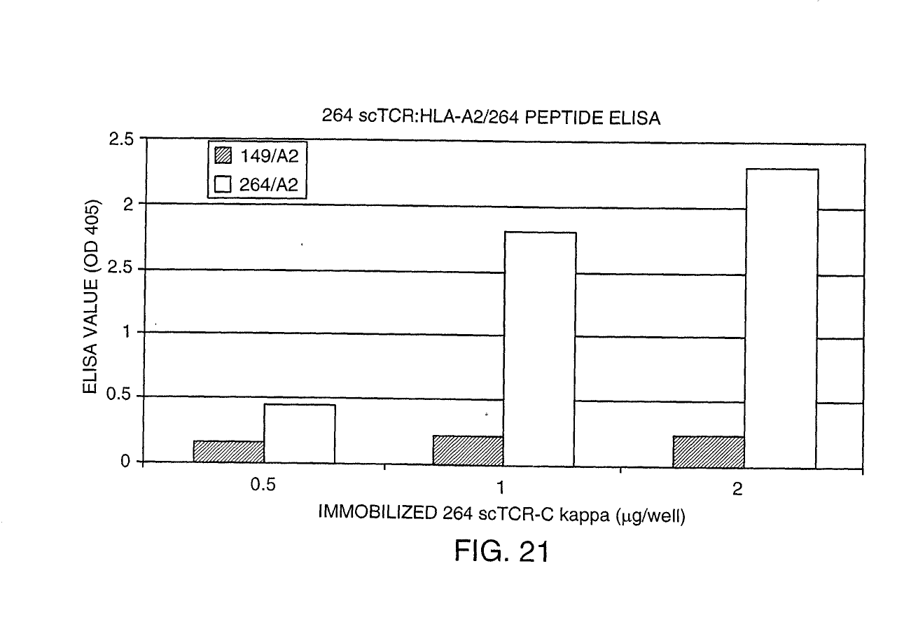

Based on these findings, an ELISA was developed using the HLA-A2/264 tetramer technology to demonstrate TCR:MHC/peptide interaction. In this assay, the 264 scTCR-C kappa fusion protein was coated at 2, 1 and 0.5 μg/well onto a 96 well plate. The plate was then washed and probed with 264/HLA-A2 tetramer. The results are shown in FIG. 21 and suggest that the HLA-A2 tetramer containing the 264 peptide, but not the 149 peptide, bound to the immobilized 264 scTCR-C kappa protein in a peptide specific manner. This study demonstrates a “proof of principle” for using a protein-based ELISA to detect the interaction of TCR and MHC/peptide molecules. The results described here suggest that this assay format could be applied to other TCR:MHC/peptide pairs to establish a protein-based assay approach to identify agonist and antagonist compounds by high throughput screening methods.

For example, the purified 264 scTCR-C kappa fusion protein is coated in 50 μl PBS onto wells of a 96 well microtiter. The amount of 264 scTCR-C kappa fusion protein required to generate a linear response will be determined experimentally. After 24 hours, the coat solution is removed and the wells receive 50 μl media (IMDM with 10% FBS) with 2.5% dimethyl sulfoxide or 50 μl media containing the test compound in 2.5% DMSO. The HLA-A2/264 tetramer (in 50 μl media) is then added. Following a 30 minute incubation at room temperature, the protein solution is removed and the wells are washed with Tris buffered saline with 1% Triton X-100. Rabbit anti-streptavidin antisera is added to each well. After a 20 minute incubation at room temperature, the wells are washed and goat anti-rabbit Ig antisera conjugated to HRP added. After 30 min at room temperature, the well are washed and ABTS substrate is added. Substrate reaction is developed and the absorbance read at 405 nm. The absorbance reading will correlate with the amount of HLA-A2/264 tetramer bound to the 264 scTCR-coated well.

The ability of a compound to antagonize interactions between the MHC/peptide complex and the recombinant TCR will be measured as a decrease in the absorbance read at 405 run. Assays in which anti-TCR Ab is used instead of the MHC/peptide complex will be carried out to determine whether the inhibitory compounds act by antagonizing TCR:MHC/peptide complex interactions or through general inhibition of protein:protein interaction. Compounds that inhibit binding of the MHC/peptide complex but do not alter binding of the anti-TCR antibody will be pursued as lead compounds. Assays using other pairs of TCR and MHC/peptide molecules will be carried out to determine the specificity of the inhibitory compounds.

The ability of a compound to increase interactions between the MHC/peptide complex and the recombinant TCR will be measured as an increase in the amount absorbance read at 405 μm. As described above, assays using other pairs of TCR and MHC/peptide molecules or anti-TCR antibodies will be carried out to determine the specificity of the stimulatory compounds. Since the HLA-A2/p53 264-272 peptide complex represents a tumor antigen, compounds that specifically stimulate the TCR interaction with this antigen may prove useful in the development of therapeutic anti-cancer agents. Similar screening methods could be developed to identify compounds that stimulate specific interactions between TCR and MHC antigen complexes in which the peptide is derived from an infectious pathogen. The ultimate goal of such a method would be the isolation of anti-infectious agents.

### EXAMPLE 17

### Development of Protein-Based Screening Methods for Identifying Compounds that Inhibit TCR:MHC/Peptide Interactions Associated with Human Autoimmune Diseases.

The objective of this example was to develop a protein-protein binding assay using recombinant human TCR and human MHC antigen reagents associated with human autoimmune disease. By establishing a protein-based assay, a high throughput screen could be developed to identify compounds that directly antagonize autoimmune TCR:MHC/peptide interactions. These compounds could potentially lead to development of selective immunosuppressive agents.

The TCR derived from the E11 human T cell clone will be expressed as a recombinant three-domain single-chain soluble TCR in mammalian cells. The methods that will be used for construction of the appropriate expression vectors; mammalian cell transfection and selection; and production and purification of the sc-TCR are described in Example 6 and in Weidanz, et al. 1998. J. Immunol. Methods 221:59, U.S. patent application Ser. Nos. 08/813,731 and 08/943,086.

An ELISA could be developed using the E11 scTCR and the scDR2/MBP-Ig (see example 4) to demonstrate autoimmune TCR:MHC/peptide interaction. In this assay, the E11 scTCR protein is coated onto a 96 well plate. The plate is then washed and probed with scDR2/MBP-Ig. Following a wash step, the bound scDR2/MBP-Ig is probed with goat anti-human IgG-HRP and detected by TMB substrate development. Absorbance at 450 nm will determine the amount of scDR2/MBP-Ig bound by the E11 scTCR coated well. This study will demonstrate a “proof of principle” for using a protein-based ELISA to detect the interaction of autoimmune TCR and MHC/peptide and will establish a protein-based assay approach to identify antagonist compounds by high throughput screening methods.

For example, purified E11 scTCR protein is coated in 50 μl PBS onto wells of a 96 well microtiter. The amount of E11 scTCR fusion protein required to generate a linear response will be determined experimentally. After 24 hours, the coat solution is removed and the wells receive 50 μl media (IMDM with 10% FBS) with 2.5% dimethyl sulfoxide or 50 μl media containing the test compound in 2.5% DMSO. The scDR2/MBP-Ig (in 50 μl media) is then added. Following incubation at room temperature, the protein solution is removed and the wells washed with Tris buffered saline with 1% Triton X-100. Goat anti-human Ig antisera conjugated to HRP is added. After 30 min at room temperature, the well are washed and TMB substrate added. Substrate reaction is quenched with 0.18 M sulfuric acid and the absorbance read at 450 n. The absorbance reading will correlate with the amount of scDR2/MBP-Ig bound to the E11 scTCR-coated well.

The ability of a compound to antagonize interactions between the MHC/peptide complex and the recombinant TCR will be measured as a decrease in the amount absorbance read at 450 nm. Assays in which anti-TCR Ab is used instead of the MHC/peptide complex will be carried out to determine whether the inhibitory compounds act by antagonizing TCR:MHC/peptide complex interactions or through general inhibition of protein:protein interaction. Compounds that inhibit binding of the MHC/peptide complex but do not alter binding of the anti-TCR antibody will be pursued as lead compounds. Assays using other pairs of TCR and MHC/peptide molecules will be carried out to determine the specificity of the inhibitory compounds.

Compounds that specifically inhibit the autoimmune TCR interaction with its cognate MHC/peptide complex may prove useful in the development of therapeutic immunosuppressive agents.

### EXAMPLE 18

### Development of Homogeneous Screening Methods for Identifying Compounds that Inhibit TCR:MHC/Peptide Interactions Associated with Human Autoimmune Diseases.

The objective of this example was to develop a protein-protein binding homogeneous assay using recombinant human TCR and human MHC/peptide reagents associated with human autoimmune disease. Homogeneous assays have the advantage in that no separation of unbound components or washing steps are required. A number of different systems allow for homogeneous assays to be carried out as either cell-based or protein-based assays.

The assay format described in this example employs specialized beads or microspheres. Bead-based reagents are commercially available in a wide variety of formats that allow for high capacity binding/capture of recombinant proteins, efficient detection and the use of different separation and homogeneous assay methods. For many high throughput screening methods, bead-based reagents are advantageous.

For example, a homogeneous luminescent proximity assay based on the AlphaScreen reagents (Packard BioScience) can be developed using the E11 TCR and the DR2/MBP molecules to demonstrate autoimmune TCR:MHC/peptide interaction. In this assay format, two small beads are labeled with TCR and MHC/peptide complexes and brought into proximity by the TCR:MHC/peptide interaction. On laser excitation, a chemical signal generated by the ‘Donor’ bead which will go undetected without the close proximity of the ‘Acceptor’ bead. When the TCR:MHC/peptide interaction brings the beads together, a cascade of chemical reactions act to produce a greatly amplified signal. Compounds can be tested for their ability to modulate (increase or decrease) the ability of the TCR:MHC/peptide interaction generate the signal.

The TCR derived from the E1 human T cell clone will be expressed as a recombinant three-domain single-chain soluble TCR in mammalian cells as described. A birA target sequence will also be incorporated into the C terminus of the E11 scTCR using the methods described in Examples 1 and 2. The purified E11 scTCR protein will be biotinylated with biotin ligase as described in Examples 1 and 2.

For this assay, the E11 scTCR-Biotin protein is bound onto streptavidin-coated Donor beads. The scDR2/MBP-Ig (see Example 4) is bound by the anti-human IgG-coated Acceptor beads. The beads are mixed to allow association between the E11 scTCR protein and the scDR2/MBP complex. This interaction brings the Donor bead and Acceptor beads together within the proximity (<200 nm) to allow a light-induced chemical reaction to occur and generate a detectable signal. The condition necessary to optimally coat the Donor and Acceptor beads and to allow interaction between the protein-coated beads will be determined experimentally. The chemical reaction is induced in the Donor bead by light at 680 nm. Following energy transfer to the proximal Acceptor bead, the signal generated is measurable at 520-620 nm. This measurement can be made in the time-resolved mode to minimize the background effects of fluorescent compounds in the assay. This study will demonstrate a “proof of principle” for using a homogenous protein-based homogeneous assay to detect the interaction of autoimmune TCR and MHC/peptide and will establish a homogeneous assay approach to identify antagonist compounds by high throughput screening methods.

For example, the E11 scTCR Donor beads and scDR2/MBP Acceptor beads are mixed in a microtiter well to generate a detectable signal. The reaction conditions required to generate a linear response will be determined experimentally. Media with 2.5% dimethyl sulfoxide or media containing the test compound in 2.5% DMSO is added to the wells. Following laser excitation at 680 nm, the fluorescent signal is detected at 550 nm in the time resolved mode (20 msec delay) using a AlphaQuest-HST Microplate Analyzer (Packard). The fluorescent signal will correlate with the amount of scDR2/MBP Acceptor bead bound to the E11 scTCR Donor bead.

The ability of a compound to antagonize interactions between the MHC/peptide complex and the TCR will be measured as a decrease in the amount fluorescent signal at 550 nm. Assays in which anti-TCR Ab is used instead of the MHC/peptide complex will be carried out to determine whether the inhibitory compounds act by antagonizing TCR:MHC/peptide complex interactions or through general inhibition of protein:protein interaction. Compounds that inhibit binding of the MHC/peptide complex but do not alter binding of the anti-TCR antibody will be pursued as lead compounds. Assays using other pairs of TCR and MHC/peptide molecules will be carried out to determine the specificity of the inhibitory compounds. Compounds that specifically inhibit the autoimmune TCR interaction with its cognate MHC/peptide complex may prove as useful in the development of therapeutic immunosuppressive agents.

### EXAMPLE 19

### Development of Protein-Based Screening Methods for Identifying Compounds that Augment and Inhibit TCR:MHC Antigen Interactions.

The objective of this example was to develop a protein-protein binding assay using recombinant TCR and MHC antigen reagents. By establishing a protein-based assay, a high throughput screen could be developed to identify compounds that directly agonize or antagonize the target TCR:MHC/peptide interaction. The advantage to such a screening assay compared to the cell-based assay would be the reduction in the number of “false positive” compounds detected that are cytotoxic or nonspecifically affect cellular responses. The approach taken was to establish an ELISA format using purified recombinant TCR and MHC antigen complexes.

Briefly, cDNA encoding the 264 TCR (peptide fragment 264-272 from wild-type human tumor suppressor protein, p53 and restricted by HLA-A2) was provided by Dr. Linda Sherman (Scripps Research Institute, La Jolla, Calif.). Amplification of the Vα3.1 and the Vβ3.0 genes was carried out as described in the pending U.S. patent application Ser. No. 08/813,731 (Fusion Proteins Comprising Bacteriophage Coat Protein and A Single-Chain T cell Receptor” and Ser. No. 08/943,086 (“Fusion Proteins Comprising Single-Chain T Cell Receptor and an Immunoglobulin Light Chain Constant Region”) except that the oligonucleotides used were specific for the 264 TCR. The cloning of the 264 TCR as a three-domain scTCR-C kappa fusion protein was carried out in the same manner as described for the DO11.10 TCR (as described in the pending U.S. patent applications—see above). The 264 scTCR-C kappa fusion vector was transfected into CHO cells for expression of soluble protein in the culture media. The 264 scTCR protein was then purified to homogeneity on an affinity column as confirmed by SDS-PAGE analysis.

Separately, a vector system used to produce the HLA-A2 and beta-2 microglobulin molecules was provided by Dr. John Altman (Emory University, Atlanta, Ga.) and has been described in Altman et al. (Altman, J. D., P. A. H. Moss, P. J. R. Goulder, D. H. Barouch, M. G. McHeyzer-Williams, J. I. Bell, A. J. McMichael & M. M. Davis. 1996. Science 274:94-96). To produce HLA-A2 molecules loaded with the peptide of interest, a protocol by Dr. Mark Davis (Stanford University, Palo Alto, Calif.) (see Altman et al. 1996. Science 274:94-96) was followed that resulted in the formation of functional molecules. Correctly folded peptide/HLA-A2 complex was isolated on a Sephadex-200 size exclusion column as a single peak migrating at 50 kD. The protein complex was then biotinylated using biotin ligase (Avidity, Denver, Colo.) to attach a single biotin moiety to a C-terminal BirA sequence. Biotinylated A2 complexes were multimerized using streptavidin, a biotin specific protein capable of binding up to four biotin residues per molecule of streptavidin. Using the HLA-A2 and the streptavidin protein at a molar ratio of 4:1 respectively, tetrameric HLA-A2/peptide complexes were generated. The advantage of using the tetramer (compared to the monomer) is the greater binding avidity displayed by the tetramer when interacting with TCR. In support of this, protein binding studies using BIAcore instrument showed that the soluble tetrameric HLA-A2/264 peptide complexes bound to immobilized 264 scTCR-C kappa fusion protein to a greater extent than the monomeric HLA-A2/264 peptide complexes.

Based on these findings, an ELISA was developed using the HLA-A2/264 tetramer technology to demonstrate TCR:MHC/peptide interaction. In this assay, the 264 scTCR-C kappa fusion protein was coated at 2, 1 and 0.5 μg/well onto a 96 well plate. The plate was then washed and probed with 264/HLA-A2 tetramer. The results are shown in FIG. 21 and suggest that the HLA-A2 tetramer containing the 264 peptide, but not the 149 peptide, bound to the immobilized 264 scTCR-C kappa protein in a peptide specific manner.

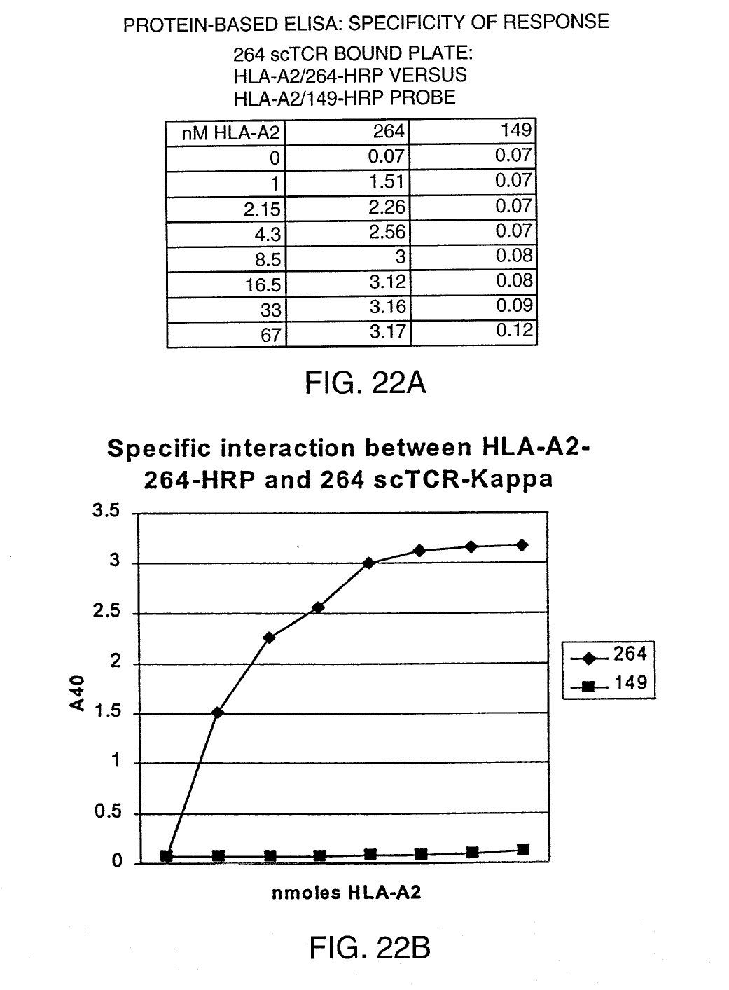

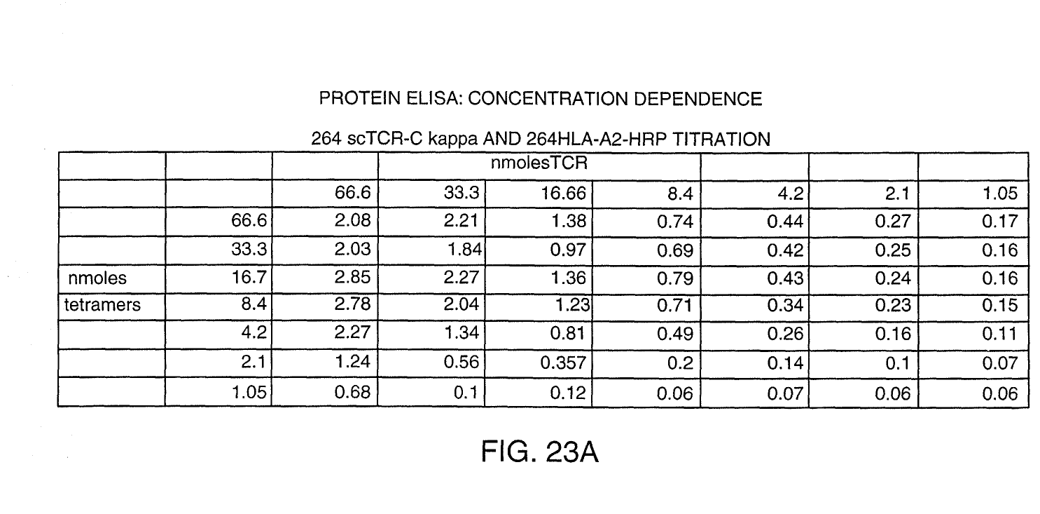

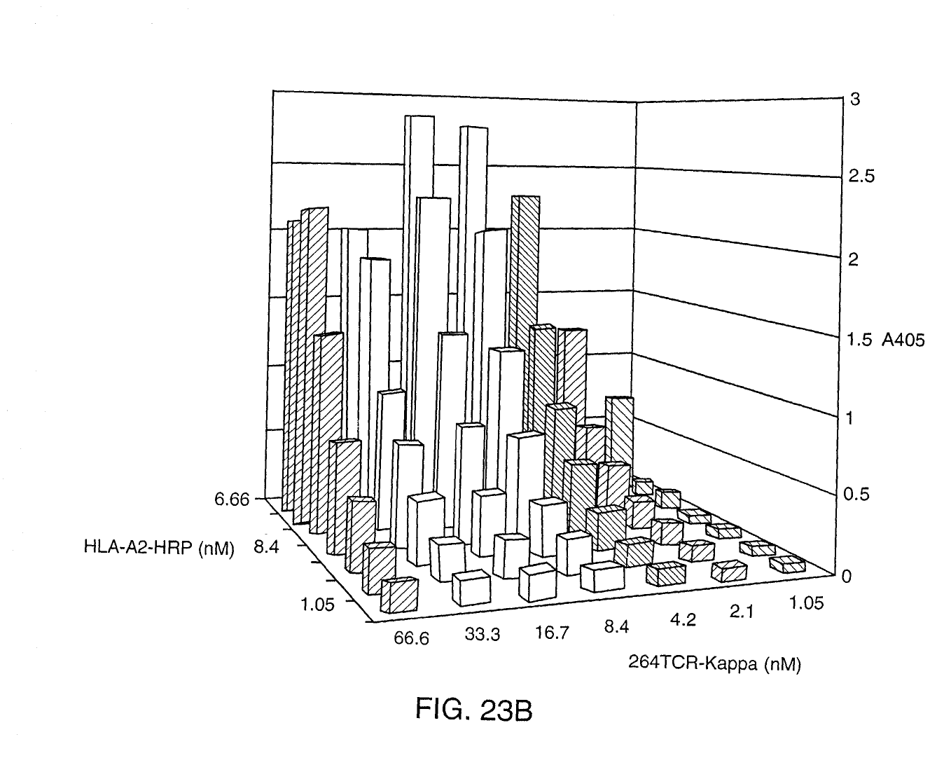

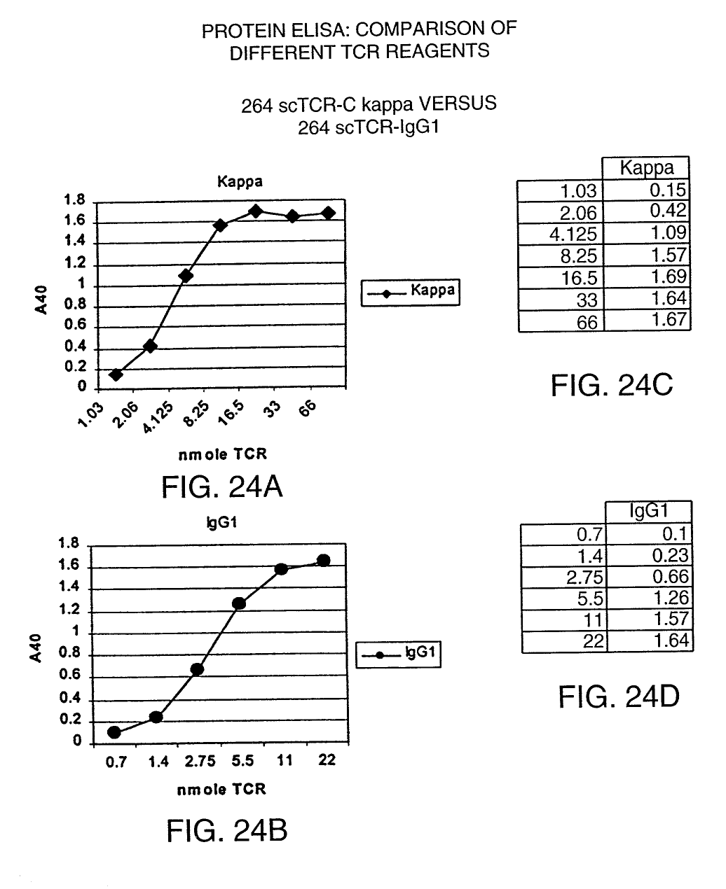

A second ELISA was developed using the HLA-A2/264 or HLA-A2/149 tetramer technology. For this assay, the tetramers were prepared as described above, except that the biotinylated HLA-A2 complexes were multimerized using streptavidin-peroxidase (Kirkegaard & Perry Laboratories) to form HLA-A2/264-HRP or HLA-A2/149-HRP tetramers. The labeled tetramers were then used as a probe to detect plate-bound TCR. To immobilize the TCR, the anti-TCR beta-constant domain specific antibody H57 (BD Pharmingen) was coated at 250 ng/well in 50 μl PBS onto wells of a 96 well microtiter plate by overnight incubation at 4° C. The antibody was removed and the wells blocked with 10% serum in PBS for 1 hour. The wells were then washed 3 times with 200 μl of Tris-buffered saline with 1% Triton X-100. The purified 264 scTCR-C kappa fusion protein was added in 50 μl PBS with 1% serum. After an hour at room temperature, the wells were washed and the HLA-A2/264-HRP (or control HLA-A2/149-HRP) tetramers were added in 50 μl PBS with 1% serum. After 2 hours at room temperature, the wells were washed and 100 μl of ABTS substrate was added and color was allowed to develop for 20 minutes. The substrate reaction was read at an absorbance of 405 nm and represents the amount of labeled MHC/peptide bound by the TCR fusion protein. The results of these studies showed specific interactions between the 264 scTCR and the HLA-A2/264-HRP probe but no interaction between the 264 scTCR and the control HLA-A2/149-HRP probe (FIGS. 22A and 22B). The amounts of 264 scTCR-C kappa fusion protein and HLA-A2.264-HRP probe required to generate a linear response was determined experimentally. Matrix studies of increasing concentration of 264 scTCR verses increasing concentration of HLA-A2/264-HRP were also carried out to determine the linear range of the response (FIGS. 23A and 23B). In another study, comparisons were made between purified 264 scTCR-IgG1 fusion protein and 264 scTCR-C kappa fusion protein. Both proteins were found to be equivalent in their ability to interact with the HLA-A2/264-HRP probe (FIGS. 24A-D).

These studies demonstrate a “proof of principle” for using a protein-based ELISA to detect the interaction of TCR and MHC/peptide molecules. The results described here suggest that this assay format could be applied to other TCR:MHC/peptide pairs to establish a protein-based assay approach to identify agonist and antagonist compounds by high throughput screening methods.

For example, the 264 scTCR-C kappa:HLA-A2/264-HRP ELISA described above was used to screen a chemical library for compounds that inhibited TCR:MHC/peptide interaction. In this experiment, 80 different compounds were individually tested at 50 μM by addition with the HLA-A2/264-HRP probe to the 264 scTCR-coated wells. The ELISA was carried out as described above. The ability of each compound to inhibit the ELISA reaction was determined. One compound was found to inhibit the substrate reaction by 80% at 50 μM and to have an ID₅₀ of 2 μM. These results indicate the feasibility of using such a protein-based assay to identify compounds that antagonize interactions between the TCR and MHC antigen.

In another example, the purified 264 scTCR-C kappa fusion protein is coated in 50 μl PBS onto wells of a 96 well microtiter. The amount of 264 scTCR-C kappa fusion protein required to generate a linear response will be determined experimentally. After 24 hours, the coat solution is removed and the wells receive 50 μl media (IMDM with 10% FBS) with 2.5% dimethyl sulfoxide or 50 μl media containing the test compound in 2.5% DMSO. The HLA-A2/264 tetramer (in 50 μl media) is then added. Following a 30 minute incubation at room temperature, the protein solution is removed and the wells are washed with Tris buffered saline with 1% Triton X-100. Rabbit anti-streptavidin antisera is added to each well. After a 20 minute incubation at room temperature, the wells are washed and goat anti-rabbit Ig antisera conjugated to HRP added. After 30 min at room temperature, the well are washed and ABTS substrate is added. Substrate reaction is developed and the absorbance read at 405 nm. The absorbance reading will correlate with the amount of HLA-A2/264 tetramer bound to the 264 scTCR-coated well.

The ability of a compound to antagonize interactions between the MHC/peptide complex and the recombinant TCR will be measured as a decrease in the absorbance read at 405 nm. Assays in which anti-TCR Ab is used instead of the MHC/peptide complex will be carried out to determine whether the inhibitory compounds act by antagonizing TCR:MHC/peptide complex interactions or through general inhibition of protein:protein interaction. Compounds that inhibit binding of the MHC/peptide complex but do not alter binding of the anti-TCR antibody will be pursued as lead compounds. Assays using other pairs of TCR and MHC/peptide molecules will be carried out to determine the specificity of the inhibitory compounds.

The ability of a compound to increase interactions between the MHC/peptide complex and the recombinant TCR will be measured as an increase in the amount absorbance read at 405 nm. As described above, assays using other pairs of TCR and MHC/peptide molecules or anti-TCR antibodies will be carried out to determine the specificity of the stimulatory compounds. Since the HLA-A2/p53 264-272 peptide complex represents a tumor antigen, compounds that specifically stimulate the TCR interaction with this antigen may prove useful in the development of therapeutic anti-cancer agents. Similar screening methods could be developed to identify compounds that stimulate specific interactions between TCR and MHC antigen complexes in which the peptide is derived from an infectious pathogen. The ultimate goal of such a method would be the isolation of anti-infectious agents.

All references disclosed herein are incorporated by reference.

While the invention has been described with reference to specific embodiments, modifications and variations of the invention may be constructed without departing from the scope of the invention, which is defined, e.g., in the following claims.

What is claimed is:

1. A method for identifying compounds that modulate an immune complex comprising a T cell receptor (TCR) and a major histocompatibility complex (MHC) antigen, the method comprising:
a) contacting a first TCR molecule and MHC antigen molecule in the presence or absence of at least one test compound, the contacting being under conditions sufficient to bind the TCR and the MHC antigen molecules specifically as an immune complex,
b) detecting presence of the immune complex in the presence and absence of the test compound,
c) selecting a test compound that alters specific binding between the TCR and MHC antigen molecules; and
d) identifying the selected compound as being capable of modulating the immune complex.

2. The method of claim 1, wherein the MHC antigen is an MHC based-molecule comprising of an MHC/peptide complex, MHC/superantigen complex, MHC/lipid (or glycolipid) complex, or an alloreactive or xenoreactive MHC molecule.

3. The method of claims 1 and 2, wherein at least one of the TCR or MHC components is a heterodimer.

4. The method of claim 3, wherein the heterodimer is naturally-occurring or recombinant.

5. The method of claims 1-4, wherein the TCR molecule is a recombinant single-chain (sc-) molecule.

6. The method of claims 1-5, wherein the TCR molecule is fully soluble.

7. The method of claims 1-5, wherein the TCR molecule is expressed by cells as a surface molecule.

8. The method of claim 7, wherein the TCR molecule comprises a transmembrane domain.

9. The method of claims 1-8, wherein the TCR molecule is multivalent.

10. The method of claims 1-9, wherein the TCR molecule comprises at least a portion of a mammalian immunoglobulin molecule.

11. The method of claim 10, wherein the fused immunoglobulin molecule comprises a constant domain; or a functional fragment thereof.

12. The method of claim 11, wherein the constant domain comprises at least a portion of a κ or λ light chain constant domain (CL).

13. The method of claim 11, wherein the constant domain is a heavy chain constant domain.

14. The method of claims 10-13, wherein the immunoglobulin molecule has an IgG, IgM, IgA or a chimeric isotype.

15. The method of claims 14, wherein the mammalian heavy chain has an IgG isotype and comprises at least a CH2-CH3 domain.

16. The method of claim 15, wherein the mammalian IgG heavy chain comprises a CH1-CH2-CH3 domain.

17. The method of claims 14, wherein the mammalian heavy chain has an IgM isotype and comprises a CH2-CH3-CH4 domain.

18. The method of claim 10, wherein the mammalian immunoglobulin molecule comprises or consists of murine or human sequence.

19. The method of claims 9-18, wherein the TCR molecule further comprises at least one tag for joining two or more TCR molecules together.

20. The method of claim 19, wherein the TCR molecule is capable of forming a dimer, trimer, tetramer or higher order complex.

21. The method of claims 1-20, wherein the TCR molecule comprises at least a portion of a fused mammalian CD3ζ sequence.

22. The method of claim 21, wherein the fused mammalian CD3ζ sequence comprises a transmembrane and a cytoplasmic domain; or a functional fragment thereof.

23. The method of claims 1-22, wherein the MHC component of the MHC antigen is class I, class II, or a combination thereof.

24. The method of claim 23, wherein the MHC component of the MHC antigen is a heterodimer or a recombinant single-chain molecule.

25. The method of claim 24, wherein the MHC antigen molecule is an MHC/peptide complex, each MHC antigen molecule comprising a fused presenting peptide.

26. The method of claim 24, wherein the MHC antigen molecule is an MCH/peptide complex, the method further comprises loading the MHC component with a suitable presenting peptide.

27. The method of claim 26, wherein the loading of the presenting peptide is performed prior to or during the contacting step.

28. The method of claims 1-27, wherein the MHC antigen molecule is fully soluble.

29. The method of claims 1-28, wherein the MHC antigen molecule is expressed by cells as a surface molecule.

30. The method of claim 29, wherein the MHC antigen molecule comprises at least a portion of a transmembrane domain.

31. The method of claims 1-30, wherein the MHC antigen molecule is multivalent.

32. The method of claims 1-31, wherein the MHC antigen molecule comprises at least a portion of a fused mammalian immunoglobulin molecule.

33. The method of claim 32, wherein the fused immunoglobulin molecule of the MHC antigen molecule comprises a constant domain; or a functional fragment thereof.

34. The method of claim 33, wherein the constant domain comprises at least a portion of a κ or λ light chain constant domain (C_{L}).

35. The method of claim 33, wherein the constant domain is a heavy chain constant domain.

36. The method of claims 32-35, wherein the immunoglobulin molecule has an IgG, IgM, IgA or a chimeric isotype.

37. The method of claims 32-36, wherein the mammalian heavy chain has an IgG isotype and comprises at least a CH2-CH3 domain.

38. The method of claim 37, wherein the mammalian IgG heavy chain comprises a CH 1-CH²-CH³ domain.

39. The method of claims 32-36, wherein the mammalian heavy chain has an IgM isotype and comprises a CH2-CH3-CH⁴ domain.

40. The method of claim 32, wherein the mammalian immunoglobulin molecule comprises or consists of murine or human sequence.

41. The method of claims 31-40, wherein the MHC antigen molecule further comprises at least one tag for joining two or more MHC antigen molecules together.

42. The method of claim 41, wherein the MHC antigen molecule is capable of forming a dimer, trimer, tetramer or higher order complex.

43. The method of claims 1-42, wherein the MHC antigen molecule comprises at least a portion of a fused mammalian CD3 ζ sequence.

44. The method of claim 43, wherein the fused mammalian CD3 ζ sequence comprises a transmembrane and a cytoplasmic domain; or a functional fragment thereof.

45. The method of claims 1-5, and 7-44, wherein the TCR molecule is expressed by cells as a surface molecule comprising at least one transmembrane domain; or functional fragment thereof.

46. The method of claim 45, wherein the cognate MHC antigen molecule is bound to a solid support.

47. The method of claims 1-27, 29-46, wherein the MHC antigen molecule is expressed by cells as a surface molecule comprising at least one transmembrane domain.

48. The method of claim 47, wherein the TCR molecule is bound to a solid support.

49. The methods of claims 46 or 48, wherein at least one of the bound MHC antigen or TCR molecules is bound to at least one of a dendrimer, synthetic or semi-synthetic polymer.

50. The method of claim 49, wherein the solid support is a tissue culture well or plate, a test strip, chromatography matrix, electrophoretic matrix, or a bead.

51. The method of claim 50, wherein the bead is magnetic.

52. The method of claims 45 and 47, wherein the detecting step further comprises detecting at least one response from cells expressing the TCR molecule, cells expressing the MHC antigen molecule; or both, the response resulting from the stable formation of the immune complex.

53. The method of claim 52, wherein the cell response detected by the method is at least one of cell adhesion, membrane potential, intracellular or extracellular ion concentration, intracellular kinase activity, phosphatase activity, intracellular protein transport, endogenous or heterologous gene expression, protein production or secretion including the production of at least one cytokine, cell proliferation, apoptosis, RNA synthesis, or DNA synthesis.

54. The method of claim 53, wherein the method further comprises measuring production of at least one cytokine by cells expressing the TCR molecule.

55. The method of claim 54, wherein the cytokine produced by the expressing cells is interleukin-2 (IL-2).

56. The method of claims 54-55, wherein the production is measured by contacting the cytokine with a first antibody capable of specifically binding the cytokine to form a complex comprising the first antibody and the cytokine.

57. The method of claim 56, wherein the first antibody is bound to a solid support.

58. The method of claims 56-57, wherein the cytokine production is further measured by contacting the complex with a detectably-labeled second antibody capable of specifically binding the cytokine.

59. The method of claim 58, wherein presence of the detectably-labeled second antibody is indicative of the immune complex formed in the presence and absence of the test compound.

60. The method of claim 53, wherein the detected cell response is T cell activation or proliferation.

61. The method of claim 60, wherein the T cell activation is measured in a standard T cell activation assay.

62. The method of claim 45, wherein the TCR expressing cells express at least one other type of cell surface TCR molecule.

63. The method of claim 62, wherein the other type of cell surface TCR molecule comprises or consists of a second TCR molecule having a different MHC antigen binding specificity than the first TCR molecule.

64. The method of claim 63, wherein the second cell surface TCR molecule is used in the method as a control.

65. The method of claims 62-64, wherein the second TCR molecule is a naturally-occurring TCR receptor endogenous to the expressing cells.

66. The method of claims 62-64, wherein the second TCR molecule is a recombinant heterodimer or a single chain (sc-) molecule.

67. The method of claims 62-66, wherein the test compound has a negligible capacity to modulate formation of a control immune complex comprising the second TCR molecule and its cognate MHC antigen.

68. The method of claims 62-67, wherein the test compound has a negligible capacity to modulate stability of a control immune complex comprising the second TCR molecule and its cognate MHC antigen.

69. The method of claims 45-68, wherein the method further comprises co-expressing with the TCR molecule(s) with at least one cluster of differentiation (CD) molecule.

70. The method of claim 69, wherein the CD molecule is capable of potentiating at least one of the cell responses.

71. The method of claim 70, wherein the CD molecule is recombinant.

72. The method of claim 71, wherein the recombinant CD molecule co-expressed by the cells is CD4.

73. The method of claims 1-72, wherein the T cells are T cell hybridomas.

74. The method of claim 73, wherein the T cell hybridomas are capable of producing at least one cytokine.

75. The method of claim 74, wherein the cytokine is interleukin-2 (IL-2).

76. The method of claims 19-20 and 41-42, wherein the tag comprises an amino acid sequence comprising at least one biotin ligase target sequence.

77. The method of claim 76, wherein the biotin ligase target sequence comprises at least the BirA sequence.

78. The method of claims 1-77, wherein at least one of the TCR or MHC antigen molecules is detectably-labeled.

79. The method of claims 1-78, wherein the label is directly or indirectly detectable.

80. The method of claim 79, wherein the label is directly detectable and includes at least one of a fluorescent, phosphorescent, luminescent, chromogenic or chemiluminescent label; or a precursor thereof.

81. The method of claim 79, wherein the label is a radionuclide.

82. The method of claim 79, wherein the label is indirectly detectable and includes at least one of a tag or an enzyme.

83. The method of claim 82, wherein the tag specifically binds an antibody, cell receptor, protein A, protein G, avidin, streptavidin or a functional fragment thereof or is the target for specific modification by a proteolytic enzyme, protein kinase, biotin ligase or a functional fragment thereof.

84. The method of claim 82, wherein the enzyme is horseradish peroxidase (HRP), beta-galactosidase (b-gal), or alkaline phosphatase (AP).

85. The method of claims 78-84, wherein the detection is accomplished by using an ELISA detection format.

86. The method of claims 1-85, wherein the selection step further comprises identifying the test compound as having the negligible capacity to modulate formation of a control immune complex.

87. The method of claims 1-86, wherein the selection step further comprises identifying the test compound as having negligible capacity to modulate stability of a control immune complex.

88. The method of claims 86 or 87, wherein the control immune complex comprises a first TCR and an antibody capable of specifically binding the first TCR.

89. The method of claims 86 or 87, wherein the control immune complex comprises a CD3 molecule and an antibody capable of specifically binding a CD3 protein.

90. The method of claims 86 or 87, wherein the control immune complex comprises a second TCR and its cognate MHC antigen molecule.

91. The method of claim 90, wherein the second TCR molecule has a different MHC antigen binding specificity than the first TCR molecule.

92. The method of claims 90 and 91, wherein the second TCR and its cognate MHC antigen molecule are naturally-occurring or recombinant molecules.

93. The method of claims 86-92, wherein formation or stability of the control immune complex is detected by measuring production of at least one cytokine by cells expressing the TCR molecule or CD3 molecule.

94. The method of claims 1-93, wherein the MHC component of the MHC antigen molecule is a sc-MHC or HLA molecule or an MHC or HLA heterodimer.

95. The method of claim 1-94, wherein the MHC antigen is a superantigen-MHC complex comprising a M1s antigen, SEA, SEB, SEC 1, SEC2, SEC3, SED, SEE, SPE-A, SPEC, ExFT or TSST superantigen.

96. A method for identifying compounds that modulate an immune complex comprising a T cell receptor (TCR) and a major histocompatibility complex (MHC) antigen, the method comprising:
a) contacting cells expressing a first TCR molecule with an MHC antigen molecule bound to a solid support in the presence or absence of a test compound, the contacting being under conditions sufficient to bind the TCR and the bound MHC antigen molecule specifically as an immune complex,
b) detecting presence of the immune complex in the presence and absence of the test compound by measuring at least one response from the cells,
c) selecting a test compound that alters specific binding between the first TCR and bound MHC antigen molecules; and
d) identifying the selected compound as being capable of modulating the immune complex.

97. The method of claim 96, wherein cell response measured in the detection step is production of a cytokine or T cell proliferation.

98. The method of claim 97, wherein the measured cell response is production of the cytokine interleukin-2 (IL-2).

99. The method of claim 98, wherein the method further comprises binding to the solid support a first antibody or functional fragment thereof capable of specifically binding the IL-2 and forming an antibody complex.

100. The method of claim 95, wherein the method further comprises contacting the antibody complex with a detectably-labeled antibody or functional fragment thereof capable of specifically binding the IL-2.

101. The method of claim 100, wherein the method further comprises quantifying the detectable label either directly or indirectly.

102. The method of claim 101, wherein the method comprises an ELISA detection format.

103. The method of claims 96-102, wherein the cells expressing the TCR molecule further express at least one of a second (control) TCR molecule or a CD protein.

104. The method of claim 103, wherein the CD protein is CD4.

105. The method of claims 96-104, wherein the selection step further comprises identifying the test compound as being able to reduce or eliminate formation of the immune complex between the first TCR and MHC antigen molecules.

106. The method of claims 96-105, wherein the selection step further comprises identifying the test compound as having negligible capacity to modulate formation of a control immune complex comprising the second TCR molecule and it cognate MHC antigen.

107. The method of claims 96-106, wherein the T cells express a multiple sclerosis (MS) TCR molecule and the cognate MHC antigen molecule is an MHC/peptide complex comprising a myelin basic protein (MBP) peptide.

108. The method of claim 107, wherein the MS TCR molecule comprises TCR alpha and beta chains from an MBP-restricted MS T cell line.

109. The method of claim 108, wherein the MS TCR is a heterodimer or a recombinant single chain molecule.

110. The method of claims 108-109, wherein the MS T cell line is E11.

111. The method of claims 96-110, wherein the MS T cell molecule comprises a covalently linked CD3ζ sequence or a functional fragment thereof.

112. The method of claims 96-111, wherein the cognate MHC antigen molecule is a single-chain (sc-) DR2/MBP protein or functional fragment thereof.

113. The method of claims 96-112, wherein the MHC component of the MHC antigen molecule is an empty MHC class II HLA-DR2 (DRB1*1501) or a functional fragment thereof.

114. The method of claim 113, wherein the method further comprises contacting the MHC component with an MBP (amino acids 83-102) peptide and loading the MHC component with the MBP peptide to form the MHC antigen molecule.

115. The method of claim 114, wherein the loading step is performed before, after, or contemporaneous with binding to the solid support.

116. The method of claims 96-113, wherein the MHC antigen molecule is an MHC class II HLA-DR2 (DRB1*1501) complex comprising a fused MBP (amino acids 83-102) peptide.

117. The method of claims 112-116, wherein the DR2/MBP protein further comprises a covalently linked IgG heavy chain constant domain; or functional fragment thereof.

118. The method of claims 113-116, wherein MHC component of the DR2/MBP molecule is a heterodimer or recombinant single chain.

119. The method of claims 96-118, wherein the conditions sufficient to bind the TCR and the MHC molecules specifically include incubation in a suitable growth media for about 8 hours at 37° C. and in an atmosphere having about 10% carbon dioxide.

120. The method of claims 1-119, wherein the test compound is dissolved in a suitable solvent.

121. The method of claim 120, wherein the solvent is at least one of water, saline, tissue culture media, about 2.5% dimethyl sulfoxide, or a physiologically acceptable buffer or carrier.

122. The method of claims 1-121, wherein the TCR or MHC molecule is multivalent and comprises a fused immunoglobulin domain.

123. The method of claims 21, 22, 43, 44 and 111, wherein the fused CD3ζ chain comprises murine or human sequence.

124. The method of claim 1, wherein the first TCR molecule is fully soluble and the MHC antigen molecule is an MHC/peptide complex.

125. The method of claim 124, wherein the first TCR molecule is a recombinant single chain (sc-) TCR molecule and the MHC antigen is recombinant class I/peptide complex comprising the class I heavy chain and beta-2 microglobulin.

126. The method of claims 124-125, wherein the first TCR molecule is bound to a solid support.

127. The method of claims 124-126, wherein the MHC antigen molecule is bound to a solid support.

128. The method of claims 124-127, wherein the first TCR molecule and the MHC antigen molecule are bound to a solid support.

129. The method of claims 46-128, wherein prior to, during, after or contemporaneous with the binding of at least one of the TCR or MHC antigen molecules to the solid support, the solid support is contacted with the test compound.

130. The method of claims 124-129, wherein at least one of the TCR or MHC molecules is detectably labeled.

131. The method of claim 130, wherein the detectable label is biotin.

132. The method of claim 131, wherein the method further comprises contacting the solid support with a streptavidin or avidin molecule capable of specifically binding the biotin and forming a complex comprising the biotin.

133. The method of claim 132, wherein formation of the complex is detected by contacting the complex with a detectably labeled antibody capable of specifically binding the complex.

134. The method of claims 130-133, wherein the detection is accomplished by using an ELISA format.

135. The method of claims 1-134, wherein the detection is accomplished by using a homogeneous assay format.

136. The method of claims 1-135, wherein the detection is accomplished by means of optical, fluorescent, or luminescent measurement.

137. The method of claims 1-136, wherein the test compound has a negligible capacity to modulate formation of a control immune complex comprising the TCR molecule and an antibody capable of specifically binding:
a) an epitope on the TCR molecule not specifically bound by the MHC antigen molecule (anti-TCR antibody), or
b) a cluster of differentiation (CD) protein associated with the TCR molecule in a cell membrane.

138. The method of claim 137, wherein the CD protein is CD3 and the antibody is capable of specifically binding CD3 associated with the TCR molecule (anti-CD3 antibody).

139. The method of claims 1-138, wherein prior to, during, after or contemporaneous with contacting the first TCR and MHC antigen molecule at least one of the first TCR or MHC antigen molecules is contacted with the test compound.

140. A test compound identified by any one of the methods of claims 1-139.

141. The test compound of claim 140, wherein the test compound is capable of modulating formation of the immune complex between the TCR molecule and its cognate MHC antigen molecule.

142. The test compound of claims 140-141, wherein the test compound is capable of reducing or increasing the rate of formation of the immune complex between the TCR and MHC antigen molecules.

143. The test compound of claims 140-141, wherein the test compound is capable of reducing or enhancing the rate of disassociation between the TCR and MHC antigen molecules.

144. The test compound of claims 140-143, wherein the test compound is capable of reducing or increasing the avidity of the immune complex between the TCR and MHC antigen molecules.

145. The test compound of claims 140-144, wherein the test compound is capable of reducing or increasing the multimerization of the immune complex between the TCR and MHC antigen molecules.

146. The test compound of claims 140-145, wherein the test compound decreases or increases at least one cell response by about 1.5 to about 100 times relative to a suitable control.

147. The test compound of claim 140, wherein the cell response is cytokine production.

148. The test compound of claim 147, wherein the cytokine produced is interleukin-2 (IL-2).

149. The test compound of claims 146-148, wherein the suitable control is the cytokine produced in the presence of an anti-TCR or anti-CD3 antibody.

150. A pharmaceutical composition comprising at least one of the compounds of claims 140-149.

151. A method of inhibiting an immune response in a mammal, the method comprising administering a therapeutically effective amount the pharmaceutical compound of claim 150.

152. A method of stimulating an immune response in a mammal, the method comprising administering a therapeutically effective amount the pharmaceutical compound of claim 150.

153. A kit for performing at least one of the methods of claims 1-139, 151 and 152.

154. A recombinant TCR molecule comprising a mammalian CD3ζ sequence or functional fragment thereof.

155. The recombinant TCR molecule of claim 154, wherein the CD3ζ sequence or functional fragment comprises murine or human sequence.
